(19) United States
(12) Patent Application Publication
(10) Pub. No.: US 2026/0217718 A1
(43) Pub. Date: Jul. 30, 2026

(54) SOLID FORMS OF A CDK2 INHIBITOR

(71) Applicant: BLUEPRINT MEDICINES CORPORATION, Cambridge, MA (US)

(72) Inventors: Caitlin N. Kinkema, Cambridge, MA (US); Brian T. Hastings, North Reading, MA (US); Colin M. Kelly, Halifax, CA (US); Lauren MacEachern, Halifax, CA (US); David B. Miller, Waltham, MA (US); Marivel Samipillai, Halifax, CA (US); Eric Torrey, Halifax, CA (US); Goss S. Kauffman, Cambridge, MA (US); Andrew M. Poitras, South Plainfiel, NJ (US); Thorsten S. Rosner, South Plainfiel, NJ (US); Christopher J. Legacy, Cambridge, MA (US)

(21) Appl. No.: 19/145,318

(22) PCT Filed: Jan. 3, 2024
(86) PCT No.: PCT/US2024/010173

§ 371 Date: Jul. 2, 2025

Related U.S. Application Data
(60) Provisional application No. 63/478,413, filed on Jan. 4, 2023.

(51) Int. Cl. C07D 487/04; A61K 31/4985; C07C 309/04; C07C 309/30; C07C 309/35

(52) U.S. Cl. CPC C07D 487/04; A61K 31/4985; C07C 309/04; C07C 309/30; C07C 309/35; C07B 2200/13

(57) ABSTRACT

The compound N-(5-(difluoromethoxy)-1H-pyrazol-3-yl)-1-((tetrahydro-2H-pyran-4-yl)methyl)-1H-pyrazolo[3,4-b]pyrazin-6-amine (Compound (I)) can be prepared as a free base in various crystalline solid forms, and in various salt forms each having one or more solid forms. Methods of preparing specific crystalline forms of free base and salts of Compound (I) are also disclosed.

### CROSS REFERENCE TO RELATED APPLICATIONS

This application claims the benefit of and priority to U.S. Provisional Patent Application No. 63/478,413, filed on Jan. 4, 2023, the disclosure of which is hereby incorporated by reference in its entirety for all purposes.

### BACKGROUND

Cyclin-Dependent Kinase (CDK) are serine/threonine protein kinases that have a central role in cell cycle progression. CDK levels remain relatively constant throughout the cell cycle, and it is the selective activation of specific CDKs allows for the proper ordering of the steps in cell cycle progression. Activation of CDKs requires heterodimerization with regulatory subunits known as cyclins. Cell cycle deregulation is a common feature of human cancer.

Cyclin-dependent kinase 2 (Cdk2) participates in a range of biological activities. CDK2 is a key cell cycle regulator, active from the late G₁-phase and throughout the S-phase. CDK2 is involved in DNA damage response (DDR) through the homologous recombination (HR) pathway. CDK2 also regulates aspects of apoptotic pathways. Cyclin E1 (CCNE1), cyclin E2 (CCNE2), cyclin A1 (CCNA1), and cyclin A2 (CCNA2), along with p21Cip1/Waf1, p27Kip1 and p57Kip2 (the cyclin dependent kinase inhibitors of the cyclin-CDK2 complex) are the main regulators of CDK2 activity. In cancer, dysregulation of the binding of CDK2 by cyclin E1, E2, A1, or A2 or the activity of the cyclin-dependent kinase inhibitor proteins may occur. (See S. Tadesse et al., Drug Discovery Today, Volume 25. Number 2 Feb. 2020)

The dysregulation of CDK2 can occur through several mechanisms. Amplification and/or overexpression of CCNE1 has been identified occurring in ovarian and breast cancer (See Scaltriti, M. et al., Proc. Natl Acad. Sci. USA 108, 3761-3766 (2011) and Etemadmoghadam, D. et al. Proc. Natl Acad. Sci. USA 110, 19489-19494 (2013). Poor outcomes in gastric, endometrial, and other cancers have been associated with overexpression and/or amplification of CCNE1 (See Ooi et al. Hum Pathol. (2017) 61:58-67, and Noske et al, Oncotarget (2017) 8: 14794-14805).

While these findings indicate CDK2 is a potential target for cancers with deregulated CDK2 activity, no agents selectively targeting CDK2 have been approved to date. Therefore, there is a need to develop new CDK2 inhibitors.

The structure of one of the inhibitors, referred to herein as “Compound (I)” or N-(5-(difluoromethoxy)-1H-pyrazol-3-yl)-1-((tetrahydro-2H-pyran-4-yl)methyl)-1H-pyrazolo[3,4-b]pyrazin-6-amine is shown below:

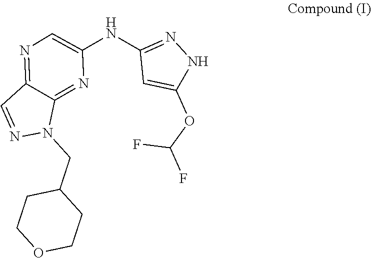 [SMILES: FC(F)Oc1cc(Nc2cnc3cnn(CC4CCOCC4)c3n2)n[nH]1]

or a pharmaceutically acceptable salt thereof.

There is a need to develop new salt forms and/or solid forms of Compound (I) that are suitable to large scale manufacture, formulation, and commercialization.

### SUMMARY

In a first aspect, the present disclosure provides a crystalline solid form of the free base of Compound (I), wherein Compound (I) is represented by the following structural formula:

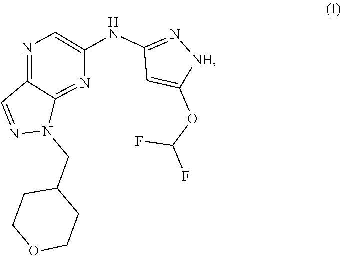 [SMILES: FC(F)Oc1cc(Nc2cnc3cnn(CC4CCOCC4)c3n2)n[nH]1]

wherein the crystalline solid form is referred to as crystalline solid Form A and is characterized by an X-ray powder diffraction (XRPD) pattern comprising three, four, or five peaks at the diffraction angles (20) selected from the group consisting of 5.9°±0.2, 17.6°±0.2, 18.1°±0.2, 23.1°±0.2, and 25.1°±0.2.

In another aspect, the present disclosure provides a crystalline solid form of the free base of Compound (I), wherein the crystalline solid form is referred to as crystalline solid Form A and is characterized by an XRPD pattern comprising three, four, or five peaks at the diffraction angles (2θ) selected from the group consisting of 5.9°±0.2, 17.6°±0.2, 21.4°±0.2, 25.1°±0.2, and 27.3°±0.2.

In another aspect, the present disclosure provides a crystalline solid form of the free base of Compound (I), wherein Compound (I) is represented by the following structural formula:

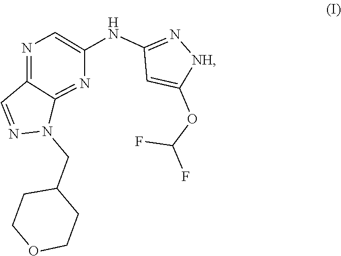 [SMILES: FC(F)Oc1cc(Nc2cnc3cnn(CC4CCOCC4)c3n2)n[nH]1]

wherein the crystalline solid form is referred to as crystalline solid Form B and is characterized by an XRPD pattern comprising three, four, or five peaks at the diffraction angles (2θ) selected from the group consisting of 5.8°±0.2, 14.9°±0.2, 17.3°±0.2, 23.9°±0.2, and 24.6°±0.2.

In another aspect, the present disclosure provides a crystalline solid form of the free base of Compound (I), wherein the crystalline solid form is referred to as crystalline solid Form B and is characterized by an XRPD pattern comprising three, four, or five peaks at the diffraction angles (2θ) selected from the group consisting of 5.8°±0.2, 17.3°±0.2, 21.5°±0.2, 25.3°±0.2, and 29.1°±0.2.

In another aspect, the present disclosure provides a crystalline solid form of the free base of Compound (I) represented by the following structural formula:

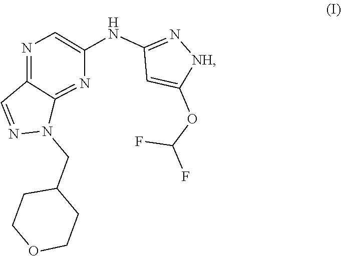 [SMILES: FC(F)Oc1cc(Nc2cnc3cnn(CC4CCOCC4)c3n2)n[nH]1]

wherein the crystalline solid form is referred to as crystalline solid Form C and is characterized by an XRPD pattern comprising three, four, five, or six peaks at the diffraction angles (2θ) selected from the group consisting of 8.7°±0.2, 10.6°±0.2, 13.4°±0.2, 15.3°±0.2, 19.0°±0.2, and 25.8°±0.2.

In another aspect, the present disclosure provides an amorphous form of the free base of Compound (I) represented by the following structural formula:

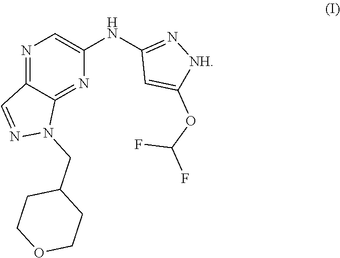 [SMILES: FC(F)Oc1cc(Nc2cnc3cnn(CC4CCOCC4)c3n2)n[nH]1]

In another aspect, the present disclosure provides a crystalline solid form of a pharmaceutically acceptable salt of Compound (I) represented by the following structural formula:

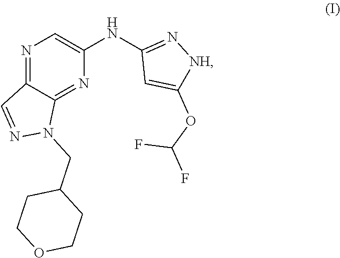 [SMILES: FC(F)Oc1cc(Nc2cnc3cnn(CC4CCOCC4)c3n2)n[nH]1]

wherein the salt is selected from the group consisting of a methanesulfonate salt, an ethanesulfonate salt, a benzenesulfonate salt, a tosylate salt, a naphthalene-2-sulfonate salt, a glycine salt, a succinate salt, a citrate salt, a tartrate salt, an adipate salt, an aspartate salt, a histidine salt, a vanillin salt, a phosphate salt, a hydrochloride salt, a hydrobromide salt, a nitrate salt, and a sulfate salt.

In another aspect, the present disclosure provides a pharmaceutical composition comprising a crystalline solid form of the free base of Compound (I) disclosed herein, the amorphous form of the free base of Compound (I) disclosed herein, or a crystalline solid form of a pharmaceutically acceptable salt of Compound (I) disclosed herein, and a pharmaceutically acceptable carrier.

In another aspect, the present disclosure provides a pharmaceutical composition comprising Compound (I) or a pharmaceutically acceptable salt thereof and a pharmaceutically acceptable carrier, wherein at least 0.1%, 0.5%, 1%, 5%, or more of the Compound (I) or pharmaceutically acceptable salt thereof by weight is present in a crystalline solid form of the free base of Compound (I) disclosed herein, the amorphous form of the free base of Compound (I) disclosed herein, or a crystalline solid form of a pharmaceutically acceptable salt of Compound (I) disclosed herein.

The present disclosure provides a method of treating a subject with cancer, comprising administering to the subject an effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition of the disclosure. In one embodiment, the cancer is uterine cancer (including uterine carcinosarcoma (UCS), uterine corpus endometrial carcinoma (UCEC)), endometrial cancer, breast cancer (including breast invasive carcinoma (BRCA), TNBC (triple negative breast cancer), HR+ breast cancer (hormone receptor positive breast cancer), ER+ breast cancer (estrogen receptor positive breast cancer), HR+HER2− breast cancer (hormone receptor positive, human epidermal growth factor 2 negative breast cancer), ER+HER2− breast cancer (estrogen receptor positive, human epidermal growth factor 2 negative breast cancer), HER2− breast cancer (human epidermal growth factor 2 negative breast cancer), HER2-low breast cancer (human epidermal growth factor 2 low breast cancer), and HER2+ breast cancer (human epidermal growth factor 2 positive breast cancer), ovarian cancer (e.g. ovarian serous cystadenocarcinoma (OV)), stomach cancer (including stomach adenocarcinoma (STAD)), gastric cancer (including gastrointestinal stromal tumor), colorectal cancer, pancreatic cancer (including pancreatic adenocarcinoma (PAAD), kidney cancer, head and neck cancer, liver cancer, prostate cancer, skin cancer, leukemia (including AML (acute myeloid leukemia)), lymphoma (including B-cell lymphoma), myelodysplastic syndromes (MDS), myeloproliferative neoplasms (MPN), sarcoma (SARC), esophageal cancer (including esophageal carcinoma (ESCA)), bladder cancer (including bladder urothelial carcinoma), lung cancer (including lung squamous carcinoma and non-small cell lung cancer, e.g., EGFRm (epidermal growth factor receptor mutant)+non-small cell lung cancer), cholangiocarcinoma, adrenocortical carcinoma (ACC), or mesothelioma. In some embodiments, the cancer is breast cancer. In one embodiment, the subject has CCNE1 amplified advanced/relapsed tumors. In one embodiment, the subject has CCNE1 amplified platinum-resistant or platinum-refectory ovarian cancer. In one embodiment, the subject has endometrial cancer (with prior platinum therapy, e.g., wherein the patient has been previously treated with a platinum therapy) that has progressed following 2 or more lines of therapies (including the platinum therapy). In one embodiment, the subject has CCNE1 amplified endometrial cancer that has failed 2 or more lines of therapies (which may include a prior platinum therapy). In one embodiment, the subject has gastric cancer (with prior platinum therapy e.g., wherein the patient has been previously treated with a platinum therapy) that has progressed following 2 or more lines of therapies (including the platinum therapy). In one embodiment, the subject has ER+HER− breast cancer that has progressed despite treatment with one or more CDK4/6 inhibitors.

In one embodiment, the cancer as described herein to be treated (e.g., the cancer as described in paragraphs [0017], [0027], [00148]-[00157], [00159], and [00161]-[00176], e.g., breast cancer) has CCNE1 amplification and/or overexpression.

In one embodiment, the cancer as described herein to be treated (e.g., the cancer as described in paragraphs [0017], [0027], [00148]-[00157], [00159], and [00161]-[00176]. e.g., breast cancer) does not have a CCNE1 amplification and/or overexpression.

The treatment method disclosed herein further comprises administering to the subject an effective amount of palbociclib (e.g., ibrance®), ribociclib, abemaciclib, tamoxifen, letrozole, olaparib (e.g., Lynparza®), niraparib, carboplatin, cisplatin, paclitaxel, gemcitabine, megestrol acetate, medroxyprogesterone acetate, capecitabine (e.g., Xeloda®), regorafenib (e.g., Stivarga®), afatinib (e.g., Gilotrif®), osimertinib (e.g., Tagrisso®), gefitinib (e.g., Iressa®), erlotinib (e.g., Tarceva®), ramucirumab (e.g., Cyramza®), an EGFR inhibitor, pralsetinib, ABT-263 (navitoclax), MK-1775 (adavosertib), BAY-1895344, berzosertib, ceralasertib, SRA-737, LY2603618 (rabusertib), or trastuzumab (e.g., Herceptin®), or combinations thereof. The EGFR inhibitor may be selected from afatinib, osimertinib, lapatinib, erlotinib, dacomitinib, poziotinib, neratinib, gefitinib JBJ-04-125-02, alflutinib (AST 2818), aumolertinib (formerly almonertinib) (HS10296), BBT-176. BI-4020. BPI-361175, BPI-D0316, CH7233163, gilitertinib, icotinib. JND-3229, lazertinib, nazartinib (EGF 816), avitinib, PCC-0208027, rezivertinib (BPI-7711), TQB3804, zorifertinib (AZ-3759), or DZD9008; an EGFR antibody such as cetuximab, panitumumab, necitumumab, HLX07, JMT101; or a bispecific EGFR and MET antibody (e.g., amivantamab ((JNJ-61186372, JNJ-372)).

The present disclosure also provides a method of inhibiting CDK2 in a subject in need thereof, comprising administering to the subject an effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition of the disclosure.

The present disclosure also provides the use of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition of the disclosure, for the preparation of a medicament for the treatment of cancers.

In another aspect, provided herein is a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition of the disclosure for use in treating cancers.

In one aspect, the present disclosure provides a method of treating a subject having, or at risk of developing, a disease or disorder associated with CDK2, comprising administering to the subject a therapeutically effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein, wherein the subject has an amplification of the CCNE1 gene and/or have an expression level of CCNE1 higher than a control expression level of CCNE1. In some embodiments, the disease or disorder associated with CDK2 is cancer.

The present disclosure also provides a method of treating a subject having, or at risk of developing, a disease or disorder associated with CDK2, comprising administering to the subject a therapeutically effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein, wherein the subject has an amplification of the CCNE1 gene and/or have an expression level of CCNE1 similar to a control expression level of CCNE1. In some embodiments, the disease or disorder associated with CDK2 is cancer.

Also provided herein is a method of treating a patient having an amplified expression level of CCNE1 and suffering from, or at risk of developing, a solid tumor cancer, comprising administering to the patient a therapeutically effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein.

The contemplated solid tumor cancer may be at least one of uterine cancer (including uterine carcinosarcoma, uterine corpus endometrial carcinoma (UCEC)), endometrial cancer, breast cancer (including breast invasive carcinoma. TNBC (triple negative breast cancer), ER (estrogen receptor)+HER2 (human epidermal growth factor 2)− breast cancer, HR (hormone receptor)+HER2 (human epidermal growth factor 2)− breast cancer, HER2− breast cancer and HER2+ breast cancer), ovarian cancer (e.g. ovarian serous cystadenocarcinoma), stomach cancer (including stomach adenocarcinoma), gastric cancer (including gastrointestinal stromal tumor), colorectal cancer, pancreatic cancer, kidney cancer, head and neck cancer, liver cancer, prostate cancer, skin cancer, lymphoma (including B-cell lymphoma), sarcoma, esophageal cancer (including esophageal carcinoma and esophageal adenocarcinoma), bladder cancer (including bladder urothelial carcinoma (BLCA)), lung cancer (including lung squamous carcinoma and non-small cell lung cancer. e.g., EGFRm (epidermal growth factor receptor mutant)+non-small cell lung cancer), cholangiocarcinoma, adrenocortical carcinoma, or mesothelioma.

In another aspect, provided herein is a method of preparing Compound (I):

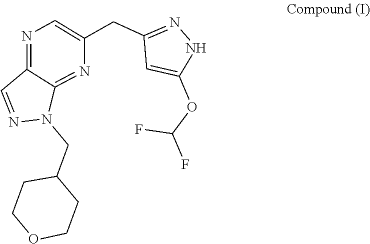 [SMILES: FC(F)Oc1cc(Cc2cnc3cnn(CC4CCOCC4)c3n2)n[nH]1]

comprising reacting a first compound represented by formula (1):

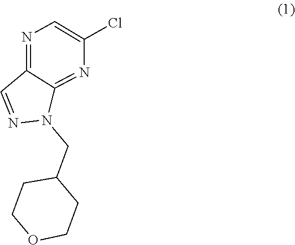 [SMILES: Clc1cnc2cnn(CC3CCOCC3)c2n1]

and a second compound represented by formula (2):

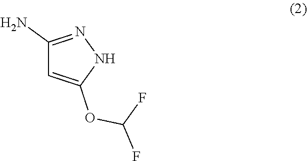

wherein reacting the first and second compound further includes a base that activates the diamine compound (2) that then reacts with the heteroaryl chloride compound (1).

### BRIEF DESCRIPTION OF THE FIGURES

FIG. 1 is a X-ray powder diffraction (XRPD) pattern designated as Pattern A obtained from Compound (I) free base designated as crystalline solid Form A, from 4-30 degrees 2-theta.

FIG. 2 is a differential scanning calorimetry (DSC) thermogram of the material analyzed in FIG. 1, obtained from a sample of Compound (I) free base designated as crystalline solid Form A.

FIG. 3 is a XRPD pattern designated as Pattern B obtained from Compound (I) free base designated as crystalline solid Form B.

FIG. 4 is a DSC thermogram of the material tested in FIG. 3, obtained from a sample of Compound (I) designated as crystalline solid Form B.

FIG. 5 is a XRPD pattern designated as Pattern C obtained from Compound (I) free base designated as crystalline solid Form C.

FIG. 6 is a DSC thermogram of the material analyzed in FIG. 5, obtained from a sample of Compound (I) free base designated as crystalline solid Form C.

FIG. 7 is a XRPD pattern designated as Pattern D obtained from Compound (I) free base designated as crystalline solid Form D.

FIG. 8 is a simultaneous thermogravimetric analysis (TGA)/DSC thermogram of the material tested in FIG. 5, obtained from a sample of Compound (I) designated as crystalline solid Form D.

FIG. 9 is a XRPD pattern designated as Pattern E+A+B obtained from Compound (I) free base designated as crystalline solid Forms E+A+B. Peaks marked with a star denote the most prominent forms of Pattern E only.

FIG. 10 is a simultaneous TGA/DSC thermogram of the material tested in FIG. 9, obtained from a sample of Compound (I) designated as crystalline solid Forms E+A+B.

FIG. 11 is a XRPD pattern designated as Pattern F obtained from Compound (I) free base designated as crystalline solid Form F.

FIG. 12 is a schematic providing exemplary conditions for converting between different crystalline solid forms of the free base of Compound (I).

FIG. 13 is a XRPD pattern designated as Pattern 6-B obtained from tosylate salt of Compound (I) designated as crystalline solid Form 6-B, from 4-30 degrees 2-theta.

FIG. 14 is a DSC thermogram of the material analyzed in FIG. 13 obtained from a sample of tosylate salt of Compound (I) designated as crystalline solid Form 6-B.

FIG. 15 is a XRPD pattern designated as Pattern 6-C obtained from tosylate salt of Compound (I) designated as crystalline solid Form 6-C, from 4-30 degrees 2-theta.

FIG. 16 is a DSC thermogram of the material analyzed in FIG. 15 obtained from a sample of tosylate salt of Compound (I) designated as crystalline solid Form 6-C.

FIG. 17 is a XRPD pattern designated as Pattern 6-D obtained from tosylate salt of Compound (I) designated as crystalline solid Form 6-D, from 4-30 degrees 2-theta.

FIG. 18 is a DSC thermogram of the material tested in FIG. 17, obtained from a sample of tosylate salt of Compound (I) designated as crystalline solid Form 6-D.

FIG. 19 is a XRPD pattern designated as Pattern 7-A obtained from ethanesulfonate salt of Compound (I) designated as crystalline solid Form 7-A, from 4-30 degrees 2-theta.

FIG. 20 is a DSC thermogram of the material analyzed in FIG. 19 obtained from a sample of ethanesulfonate salt of Compound (I) designated as crystalline solid Form 7-A.

FIG. 21 is a XRPD pattern designated as Pattern 8-A obtained from naphthalene-2-sulfonate salt of Compound (I) designated as crystalline solid Form 8-A, from 4-30 degrees 2-theta.

FIG. 22 is a DSC thermogram of the material analyzed in FIG. 21 obtained from a sample of naphthalene-2-sulfonate salt of Compound (I) designated as crystalline solid Form 8-A.

FIG. 23 is a XRPD pattern designated as Pattern 1-B obtained from HCl salt of Compound (I) designated as crystalline solid Form 1-B, from 4-30 degrees 2-theta.

FIG. 24 is a simultaneous TGA/DSC thermogram of the material tested in FIG. 23, obtained from a sample of HCl salt of Compound (I) designated as crystalline solid Form 1-B.

FIG. 25 is a XRPD pattern designated as Pattern 2-A obtained from HBr salt of Compound (I) designated as crystalline solid Form 2-A, from 4-30 degrees 2-theta.

FIG. 26 is a XRPD pattern designated as Pattern 3-A obtained from besylate salt of Compound (I) designated as crystalline solid Form 3-A, from 4-30 degrees 2-theta.

FIG. 27 is a XRPD pattern designated as Pattern 4-A obtained from mesylate salt of Compound (I) designated as crystalline solid Form 4-A, from 4-30 degrees 2-theta.

### DETAILED DESCRIPTION

Provided herein, in part, are crystalline solid forms of N-(5-(difluoromethoxy)-1H-pyrazol-3-yl)-1-((tetrahydro-2H-pyran-4-yl)methyl)-1H-pyrazolo[3,4-b]pyrazin-6-amine (herein, Compound (I)) or a pharmaceutically acceptable salt thereof, represented by the following structural formula:

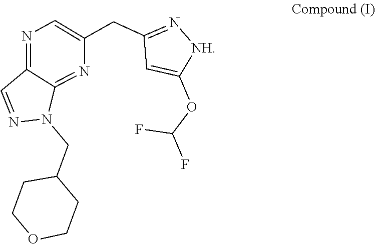 [SMILES: FC(F)Oc1cc(Cc2cnc3cnn(CC4CCOCC4)c3n2)n[nH]1]

When used alone, the term “crystalline solid Form A” refers to the crystalline polymorph Form A of Compound (I). The terms “crystalline solid Form A,” “crystalline Form A,” “Solid Form A,” “Pattern A,” “Form A,” “Form A of Compound (I),” or “Form A of N-(5-(difluoromethoxy)-1H-pyrazol-3-yl)-1-((tetrahydro-2H-pyran-4-yl)methyl)-1H-pyrazolo[3,4-b]pyrazin-6-amine,” are used interchangeably. Form A can be characterized by, for example, XRPD alone or XRPD in combination with any one or more of DSC, and TGA.

When used alone, the term “crystalline solid Form B” refers to the crystalline polymorph Form B of Compound (I). The terms “crystalline solid Form B,” “crystalline Form B,” “Solid Form B,” “Pattern B,” “Form B,” “Form B of Compound (I),” or “Form B of N-(5-(difluoromethoxy)-1H-pyrazol-3-yl)-1-((tetrahydro-2H-pyran-4-yl)methyl)-1H-pyrazolo[3,4-b]pyrazin-6-amine,” are used interchangeably. Form B can be characterized by, for example, XRPD alone or XRPD in combination with any one or more of DSC, and TGA.

When used alone, the term “crystalline solid Form C” refers to the crystalline polymorph Form C of Compound (I). The terms “crystalline solid Form C,” “crystalline Form C,” “Solid Form C,” “Pattern C,” “Form C,” “Form C of Compound (I),” or “Form C of N-(5-(difluoromethoxy)-1H-pyrazol-3-yl)-1-((tetrahydro-2H-pyran-4-yl)methyl)-1H-pyrazolo[3,4-b]pyrazin-6-amine,” are used interchangeably. Form C can be characterized by, for example, XRPD alone or XRPD in combination with any one or more of DSC, and TGA.

When used alone, the term “crystalline solid Form D” refers to the crystalline polymorph Form D of Compound (I). The terms “crystalline solid Form D,” “crystalline Form D,” “Solid Form D,” “Pattern D,” “Form D,” “Form D of Compound (I),” or “Form D of N-(5-(difluoromethoxy)-1H-pyrazol-3-yl)-1-((tetrahydro-2H-pyran-4-yl)methyl)-1H-pyrazolo[3,4-b]pyrazin-6-amine,” are used interchangeably. Form D can be characterized by, for example, XRPD alone or XRPD in combination with any one or more of DSC, and TGA.

When used alone, the term “crystalline solid Form E” refers to the crystalline polymorph Form E of Compound (I). The terms “crystalline solid Form E,” “crystalline Form E,” “Solid Form E,” “Pattern E,” “Form E,” “Form E of Compound (I),” or “Form E of N-(5-(difluoromethoxy)-1H-pyrazol-3-yl)-1-((tetrahydro-2H-pyran-4-yl)methyl)-1H-pyrazolo[3,4-b]pyrazin-6-amine,” are used interchangeably. Form E can be characterized by, for example, XRPD alone or XRPD in combination with any one or more of DSC, and TGA.

When used alone, the term “crystalline solid Form F” refers to the crystalline polymorph Form F of Compound (I). The terms “crystalline solid Form F,” “crystalline Form F,” “Solid Form F,” “Pattern F,” “Form F,” “Form F of Compound (I),” or “Form F of N-(5-(difluoromethoxy)-1H-pyrazol-3-yl)-1-((tetrahydro-2H-pyran-4-yl)methyl)-1H-pyrazolo[3,4-b]pyrazin-6-amine,” are used interchangeably. Form F can be characterized by, for example, XRPD alone or XRPD in combination with any one or more of DSC, and TGA.

As used herein, the term “crystalline” refers to a solid having a crystal structure wherein the individual molecules have a highly homogeneous regular three dimensional configuration.

Solid state ordering of solids may be determined by standard techniques known in the art, e.g., by X-ray powder diffraction (XRPD), differential scanning calorimetry (DSC), thermal gravimetric analysis (TGA), or dynamic vapor sorption (DVS). Amorphous solids can also be differentiated from crystalline solids e.g., by birefringence using polarized light microscopy. Amorphous solids consist of disordered arrangements of molecules and do not possess a distinguishable crystal lattice.

Relative intensity is calculated as a ratio of the peak intensity of the peak of interest versus the peak intensity of the largest peak. In certain embodiments, the relative intensity of the peaks may vary due to the preferred orientation of the sample. Preferred orientation in the specimen influences the intensities of various reflections so that some are more intense and others less intense, compared to what would be expected from a completely random specimen. In general, the morphology of many crystalline particles tends to give a specimen that exhibits some degree of preferred orientation in the specimen holder. This is particularly evident for needlelike or plate-like crystals when size reduction yields finer needles or platelets.

In some embodiments, Form A is at least 70%, 80%, 90%, 95%, 98%, 99%, 99.5%, or 99.9% pure. The purity of Form A is determined by dividing the weight of Form A of the Compound (I) in a composition comprising Compound (I) over the total weight of Compound (I) in the composition.

In some embodiments, Form B is at least 70%, 80%, 90%, 95%, 98%, 99%, 99.5%, or 99.9% pure. The purity of Form B is determined by dividing the weight of Form B of the Compound (I) in a composition comprising Compound (I) over the total weight of Compound (I) in the composition.

In some embodiments, Form C is at least 70%, 80%, 90%, 95%, 98%, 99%, 99.5%, or 99.9% pure. The purity of Form C is determined by dividing the weight of Form C of the Compound (I) in a composition comprising Compound (I) over the total weight of Compound (I) in the composition.

In some embodiments, Form D is at least 70%, 80%, 90%, 95%, 98%, 99%, 99.5%, or 99.9% pure. The purity of Form D is determined by dividing the weight of Form D of the Compound (I) in a composition comprising Compound (I) over the total weight of Compound (I) in the composition.

In some embodiments, Form E is at least 70%, 80%, 90%, 95%, 98%, 99%, 99.5%, or 99.9% pure. The purity of Form E is determined by dividing the weight of Form E of the Compound (I) in a composition comprising Compound (I) over the total weight of Compound (I) in the composition.

In some embodiments, Form F is at least 70%, 80%, 90%, 95%, 98%, 99%, 99.5%, or 99.9% pure. The purity of Form F is determined by dividing the weight of Form G of the Compound (I) in a composition comprising Compound (I) over the total weight of Compound (I) in the composition.

When the crystalline Compound (I) salt or free base is defined as a specified percentage of one particular crystal form of the Compound (I) salt or free base, the remainder is made up of amorphous form and/or crystal forms other than the one or more particular forms that are specified.

### Compound (I) Free Base Solid Forms

In some embodiments, a solid form disclosed herein comprises the free base of Compound (I):

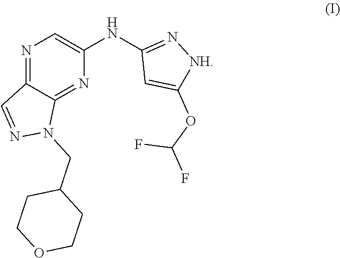 [SMILES: FC(F)Oc1cc(Nc2cnc3cnn(CC4CCOCC4)c3n2)n[nH]1]

A free base form of Compound (I) can exist in an amorphous solid form or in different solid forms, or mixtures of solid forms, which can additionally include one or more equivalents of water (e.g., anhydrous or hydrate forms). In some embodiments, a free base solid form of Compound (I) is amorphous. As provided herein, crystalline solid form(s) of Compound (I) can be identified by distinct XRPD peaks, e.g., a XRPD pattern comprising characteristic diffraction peaks at 2-theta angles. In some embodiments, the crystalline solid form of Compound (I) is a free base. In some embodiments, the crystalline solid form of Compound (I) is an anhydrous free base. There are provided herein certain crystalline solid forms of the free base of Compound (1) and related methods for preparing and using these solid form materials.

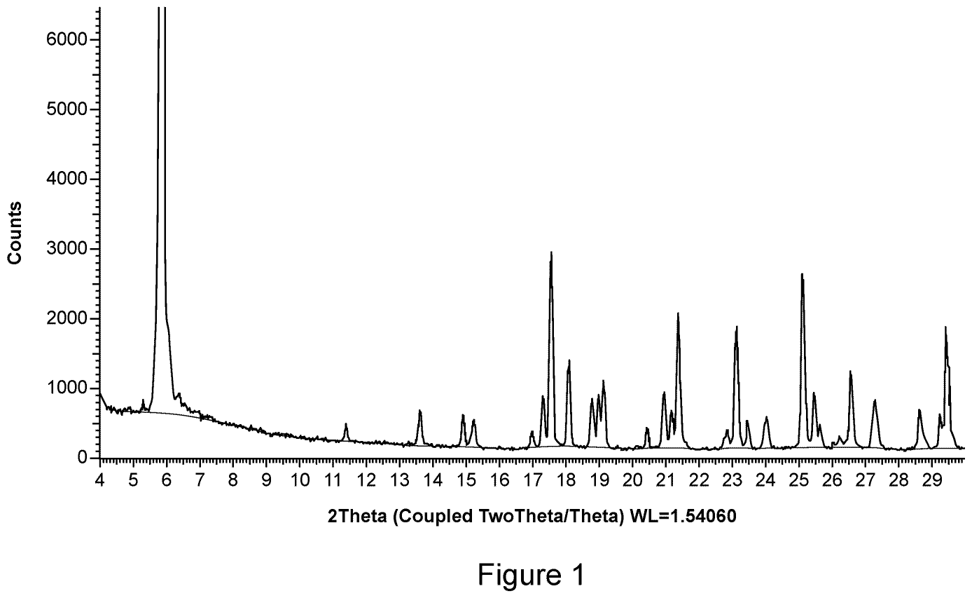

In one aspect, provided herein is a first crystalline solid form of the free base of Compound (I) designated as crystalline solid Form A. In some embodiments, Form A is anhydrous. In some embodiments, provided herein is a crystalline solid form of the free base of Compound (I), wherein Compound (I) is represented by the following structural formula:

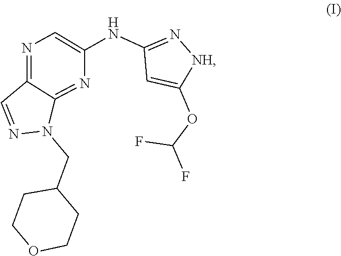 [SMILES: FC(F)Oc1cc(Nc2cnc3cnn(CC4CCOCC4)c3n2)n[nH]1]

wherein the crystalline solid form is referred to as crystalline solid Form A and is characterized by an X-ray powder diffraction (XRPD) pattern comprising three, four, or five peaks at the diffraction angles (2θ) selected from the group consisting of 5.9°±0.2, 17.6°±0.2, 18.1°±0.2, 23.1°±0.2, and 25.1°±0.2. In some embodiments, provided herein is a crystalline solid form of the free base of Compound (I), wherein the crystalline solid form is referred to as crystalline solid Form A and is characterized by an XRPD pattern comprising three, four, or five peaks at the diffraction angles (2θ) selected from the group consisting of 5.9°±0.2, 17.6°±0.2, 21.4°±0.2, 25.1°±0.2, and 27.3°±0.2. In some embodiments, crystalline solid Form A is characterized by an XRPD pattern comprising three, four, five, six, seven, eight, nine, or ten peaks at the diffraction angles (2θ) selected from the group consisting of 5.9°±0.2, 17.6°±0.2, 18.1°±0.2, 19.1°±0.2, 21.4°±0.2, 23.1°±0.2, 25.1°±0.2, 26.6°±0.2, 27.3°±0.2, and 29.4°±0.2. In some embodiments, crystalline solid Form A is characterized by an XRPD pattern comprising three, four, five, six, seven, eight, nine, ten, eleven, twelve, thirteen, fourteen, or fifteen peaks at the diffraction angles (2θ) selected from the group consisting of 5.9°±0.2, 17.3°±0.2, 17.6°±0.2, 18.1°±0.2, 18.8° 00.2, 19.0°±0.2, 19.1°±0.2, 21.0°±0.2, 21.4°±0.2, 23.1°±0.2, 25.1°±0.2, 25.5°±0.2, 26.6°±0.2, 27.3°±0.2, and 29.4°±0.2. In some embodiments, crystalline solid Form A is characterized by an XRPD pattern having characteristic peaks at the diffraction angles (2θ) at 5.9°±0.2, 17.6°±0.2, and 25.1°±0.2. In some embodiments, crystalline solid Form A is characterized by an XRPD pattern having characteristic peaks at the diffraction angles (2θ) at 5.9°±0.2, 17.6°±0.2, 21.4°±0.2, 23.1°±0.2, and 25.1°±0.2. In some embodiments, crystalline solid Form A is also characterized by an XRPD pattern substantially similar to FIG. 1.

Table 1A lists XRPD (2-theta) peaks obtained from samples of crystalline solid Form A of the free base of Compound (I). In some embodiments, crystalline solid Form A is characterized by a XRPD pattern comprising at least three, four, five, six, seven, eight, nine, ten, eleven, twelve, thirteen, fourteen, or fifteen peaks selected from Table 1A.

TABLE 1A
Peak list for Compound (I) freebase Pattern A

| 2-theta ± 0.2 (deg) | Relative intensity¹ |
|---|---|
| 5.86 | 100 |
| 6.36 | 1 |
| 11.39 | 1 |
| 13.61 | 1 |
| 14.91 | 1 |
| 15.23 | 1 |
| 16.98 | 1 |
| 17.31 | 2 |
| 17.56 | 7 |
| 18.09 | 3 |
| 18.79 | 2 |
| 18.98 | 2 |
| 19.13 | 2 |
| 20.44 | 1 |
| 20.95 | 2 |
| 21.17 | 1 |
| 21.38 | 5 |
| 22.85 | 1 |
| 23.12 | 4 |
| 23.46 | 1 |
| 24.02 | 1 |
| 25.12 | 6 |
| 25.46 | 2 |
| 25.64 | 1 |
| 26.56 | 3 |
| 27.29 | 2 |
| 28.63 | 1 |
| 29.25 | 1 |
| 29.43 | 4 |

¹Relative intensities < 1 not included

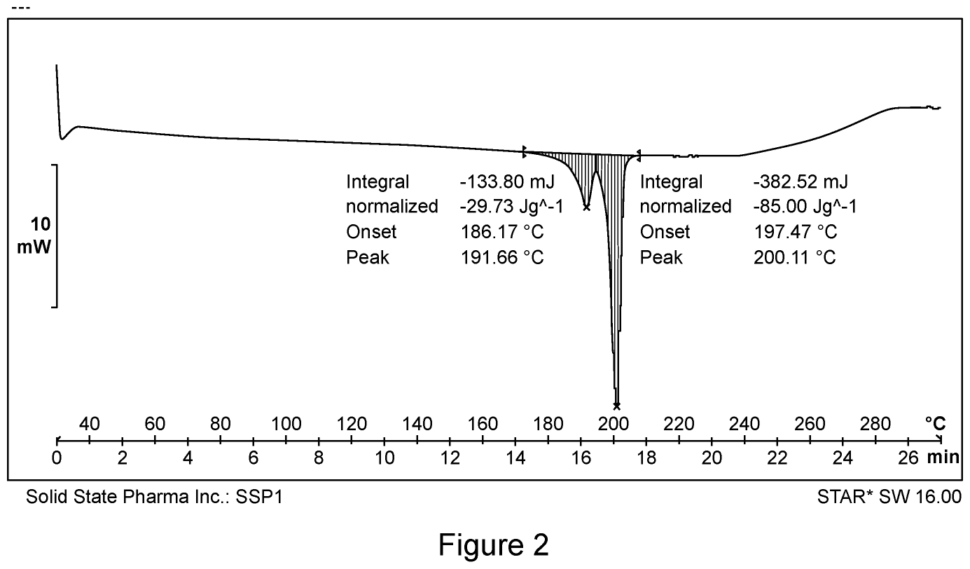

In some embodiments, crystalline solid Form A is also characterized by a differential scanning calorimetry (DSC) thermogram having two endotherms with onset peaks at 186.2±2° C. and 197.5±2° C. In some embodiments, crystalline solid Form A is also characterized by a DSC thermogram substantially similar to FIG. 2.

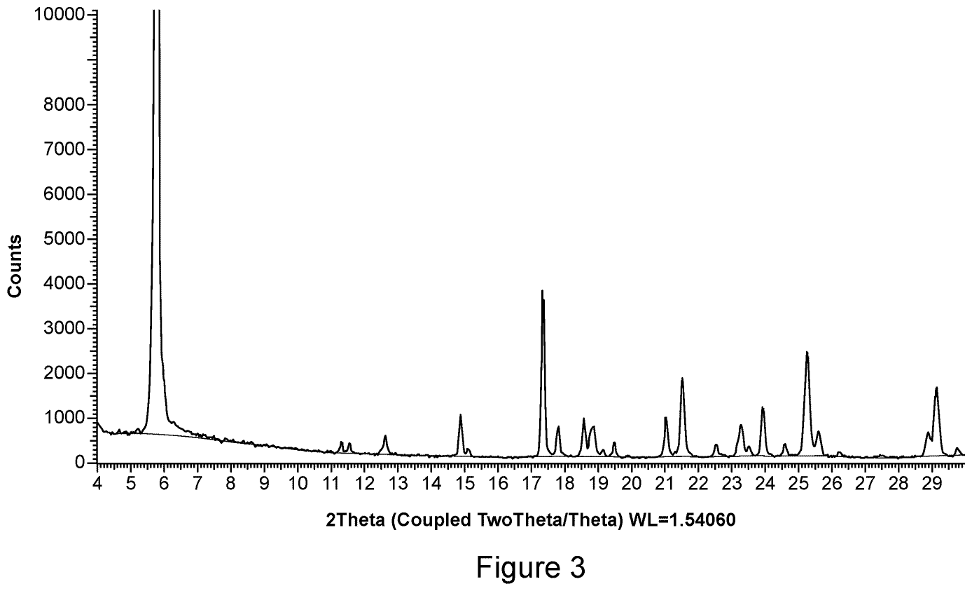

In one aspect, provided herein is a second crystalline solid form of the free base of Compound (I) designated as crystalline solid Form B. In some embodiments, Form B is anhydrous. In some embodiments, provided herein is a crystalline solid form of the free base of Compound (I), wherein Compound (I) is represented by the following structural formula:

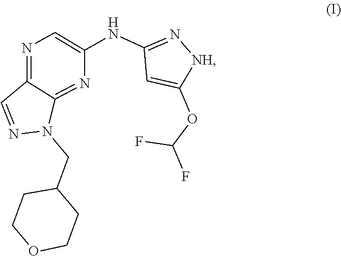 [SMILES: FC(F)Oc1cc(Nc2cnc3cnn(CC4CCOCC4)c3n2)n[nH]1]

wherein the crystalline solid form is referred to as crystalline solid Form B and is characterized by an XRPD pattern comprising three, four, or five peaks at the diffraction angles (2θ) selected from the group consisting of 5.8°±0.2, 14.9°±0.2, 17.3°±0.2, 23.9°±0.2, and 24.6°±0.2. In some embodiments, provided herein is a crystalline solid form of the free base of Compound (I), wherein the crystalline solid form is referred to as crystalline solid Form B and is characterized by an XRPD pattern comprising three, four, or five peaks at the diffraction angles (29) selected from the group consisting of 5.8°±0.2, 17.3°±0.2, 21.5°±0.2, 25.3°±0.2, and 29.1°±0.2. In some embodiments, crystalline solid Form B is characterized by an XRPD pattern comprising three, four, five, six, seven, eight, nine, or ten peaks at the diffraction angles (2θ) selected from the group consisting of 5.8°±0.2, 14.9°±0.2, 17.3°±0.2, 19.5°±0.2, 21.0°±0.2, 21.5°±0.2, 23.9°±0.2, 24.6°±0.2, 25.3°±0.2, and 29.1°±0.2. In some embodiments, crystalline solid Form B is characterized by an XRPD pattern comprising three, four, five, six, seven, eight, nine, ten, eleven, twelve, thirteen, fourteen, or fifteen peaks at the diffraction angles (2θ) selected from the group consisting of 5.8°±0.2, 12.6°±0.2, 14.9°±0.2, 17.3°±0.2, 17.8°±0.2, 18.6°±0.2, 19.5°±0.2, 21.0°±0.2, 21.5°±0.2, 23.3°±0.2, 23.9°±0.2, 24.6°±0.2, 25.3°±0.2, 28.9°±0.2, and 29.1°±0.2. In some embodiments, crystalline solid Form B is characterized by an XRPD pattern having characteristic peaks at the diffraction angles (2θ) at 5.8°±0.2, 17.3°±0.2, and 25.3°±0.2. In some embodiments, crystalline solid Form B is characterized by an XRPD pattern having characteristic peaks at the diffraction angles (2θ) at 5.8°±0.2, 17.3°±0.2, 21.5°±0.2, 25.3°±0.2, and 29.1°±0.2. In some embodiments, crystalline solid Form B is also characterized by an XRPD pattern substantially similar to FIG. 3.

Table 2A lists XRPD (2-theta) peaks obtained from samples of crystalline solid Form B of the free base of Compound (I). In some embodiments, crystalline solid Form B is characterized by a XRPD pattern comprising at least three, four, five, six, seven, eight, nine, ten, eleven, twelve, thirteen, fourteen, or fifteen peaks selected from Table 2A.

TABLE 2A
Peak list for Compound (I) freebase Pattern B

| 2-theta ± 0.2 (deg) | Relative intensity¹ |
|---|---|
| 5.77 | 100 |
| 11.30 | 1 |
| 12.62 | 1 |
| 14.87 | 2 |
| 17.34 | 8 |
| 17.80 | 1 |
| 18.57 | 2 |
| 18.84 | 1 |
| 19.48 | 1 |
| 21.03 | 2 |
| 21.52 | 4 |
| 22.53 | 1 |
| 23.28 | 2 |
| 23.93 | 2 |
| 24.60 | 1 |
| 25.26 | 5 |
| 25.60 | 1 |
| 28.89 | 1 |
| 29.13 | 3 |

¹Relative intensities < 1 not included

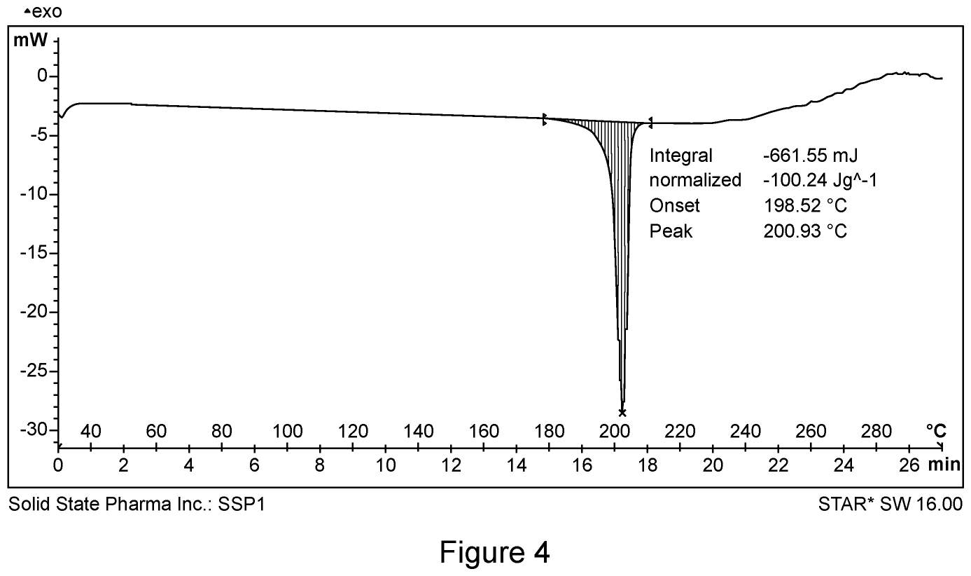

In some embodiments, crystalline solid Form B is also characterized by a DSC thermogram having a broad endotherm with an onset peak at 198.5±2° C. In some embodiments, crystalline solid Form B is also characterized by a DSC thermogram substantially similar to FIG. 4.

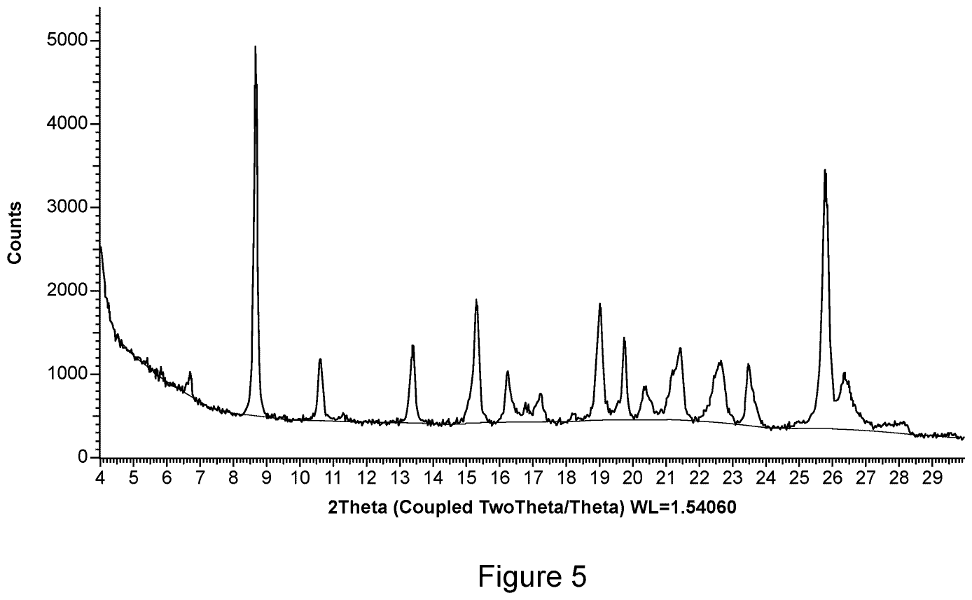

In one aspect, provided herein is a third crystalline solid form of the free base of Compound (I) designated as crystalline solid Form C. In some embodiments, crystalline solid Form C is anhydrous. In some embodiments, provided herein is a free base of Compound (I), wherein the compound is crystalline solid form of the free base of Compound (I) represented by the following structural formula:

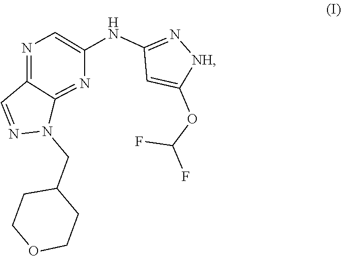 [SMILES: FC(F)Oc1cc(Nc2cnc3cnn(CC4CCOCC4)c3n2)n[nH]1]

wherein the crystalline solid form is referred to as crystalline solid Form C and is characterized by an XRPD pattern comprising three, four, five, or six peaks at the diffraction angles (2θ) selected from the group consisting of 8.7°±0.2, 10.6°±0.2, 13.4°±0.2, 15.3°±0.2, 19.0°±0.2, and 25.8°±0.2. In some embodiments, crystalline solid Form C is characterized by an XRPD pattern comprising three, four, five, six, seven, eight, nine, or ten peaks at the diffraction angles (2θ) selected from the group consisting of 8.7°±0.2, 10.6°±0.2, 13.4°±0.2, 15.3°±0.2, 16.2°±0.2, 19.0°±0.2, 19.8°±0.2, 21.4°±0.2, 22.6°±0.2, and 25.8°±0.2. In some embodiments, crystalline solid Form C is characterized by an XRPD pattern having characteristic peaks at the diffraction angles (2θ) at 8.7°±0.2, 15.3°±0.2, and 25.8°±0.2. In some embodiments, crystalline solid Form C is characterized by an XRPD pattern having characteristic peaks at the diffraction angles (2θ) at 8.7°±0.2, 15.3°±0.2, 19.0°±0.2, 19.8°±0.2, and 25.8°±0.2, In some embodiments, crystalline solid Form C is also characterized by an XRPD pattern substantially similar to FIG. 5.

Table 3A lists XRPD (2-theta) peaks obtained from samples of crystalline solid Form C of the free base of Compound (1). In some embodiments, crystalline solid Form C is characterized by a XRPD pattern comprising at least three, four, five, six, seven, eight, nine, ten, eleven, twelve, thirteen, fourteen, or fifteen peaks selected from Table 3A or 3B.

TABLE 3A
Peak list for Compound (I) free base Pattern C

| 2-theta ± 0.2 (deg) | Relative intensity¹ |
|---|---|
| 6.68 | 6 |
| 8.66 | 100 |
| 10.60 | 17 |
| 11.29 | 2 |
| 13.39 | 22 |
| 15.30 | 34 |
| 16.23 | 14 |
| 16.77 | 5 |
| 17.22 | 8 |
| 18.21 | 2 |
| 19.01 | 32 |
| 19.75 | 23 |
| 20.35 | 9 |
| 21.25 | 14 |
| 21.42 | 19 |
| 22.62 | 16 |
| 23.49 | 17 |
| 25.79 | 71 |
| 26.37 | 16 |
| 28.07 | 3 |

¹Relative intensities < 2 not included

TABLE 3B
Condensed peak list #1 for Compound (I) free base Pattern C

| 2-theta ± 0.2 (deg) | Relative intensity |
|---|---|
| 6.68 | 6 |
| 8.66 | 100 |
| 10.60 | 17 |
| 13.39 | 22 |
| 15.30 | 34 |
| 16.23 | 14 |
| 19.01 | 32 |
| 19.75 | 23 |
| 20.35 | 9 |
| 21.25 | 14 |
| 21.42 | 19 |
| 22.62 | 16 |
| 23.49 | 17 |
| 25.79 | 71 |
| 26.37 | 16 |

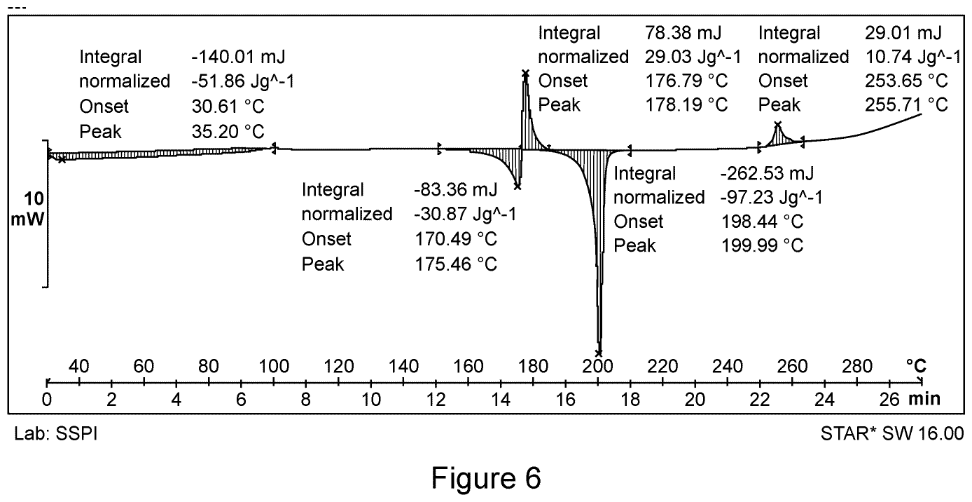

In some embodiments, crystalline solid Form C is also characterized by a DSC thermogram having three broad endotherms with onset peaks at 30.6±2° C., 170.5±2° C., and 198.4±2° C. and two broad exotherms with onset peaks at 176.8±2° C. and 253.7±2° C. In some embodiments, crystalline solid Form C is also characterized by a DSC thermogram comprising two broad endotherms with onset peaks at 170.5±2° C. and 198.4±2° C. and, optionally, two broad exotherms with onset peaks at 176.8±2° C. and 253.7±2° C. In some embodiments, crystalline solid Form C is also characterized by a DSC thermogram substantially similar to FIG. 6.

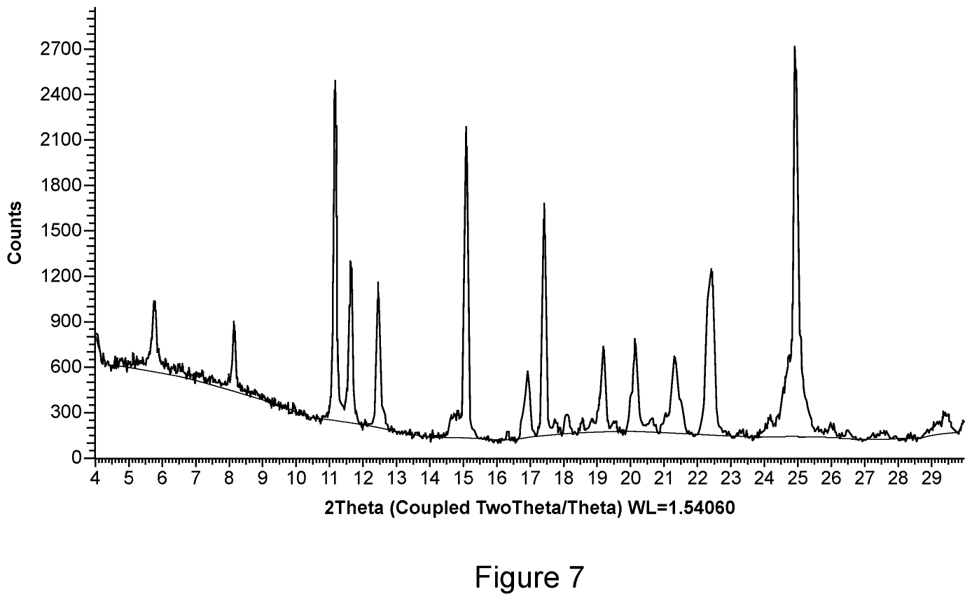

In one aspect, provided herein is a fourth crystalline solid form of the free base of Compound (I) designated as crystalline solid Form D. In some embodiments, crystalline solid Form D is a hydrate. In some embodiments, crystalline solid Form D is characterized by an XRPD pattern substantially similar to FIG. 7.

Tables 4A-4D list XRPD (2-theta) peaks obtained from samples of crystalline solid Form D of the free base of Compound (I). In some embodiments, the crystalline solid form D is characterized by a XRPD pattern comprising at least three, four, five, six, seven, eight, nine, ten, eleven, twelve, thirteen, fourteen, or fifteen peaks (up to the maximum number of peaks in each table) selected from Tables 4A, 4B, 4C, or 4D.

TABLE 4A
Peak list for Compound (I) freebase Pattern D

| 2-theta ± 0.2 (deg) | Relative intensity¹ |
|---|---|
| 5.76 | 18 |
| 8.14 | 18 |
| 11.17 | 87 |
| 11.63 | 42 |
| 12.46 | 37 |
| 14.70 | 5 |
| 14.82 | 7 |
| 15.09 | 80 |
| 16.92 | 16 |
| 17.41 | 60 |
| 17.74 | 4 |
| 18.09 | 5 |
| 18.56 | 4 |
| 18.85 | 3 |
| 19.19 | 22 |
| 20.14 | 24 |
| 20.62 | 3 |
| 21.08 | 4 |
| 21.32 | 20 |
| 22.40 | 43 |
| 24.53 | 11 |
| 24.18 | 6 |
| 24.97 | 83 |
| 24.78 | 20 |
| 24.93 | 100 |
| 26.01 | 3 |
| 29.21 | 3 |
| 29.43 | 5 |

¹Relative intensities < 3 not included

TABLE 4B
Condensed peak list #1 for Compound (I) freebase Pattern D

| 2-theta ± 0.2 (deg) | Relative intensity |
|---|---|
| 8.14 | 18 |
| 11.17 | 87 |
| 11.63 | 42 |
| 12.46 | 37 |
| 15.09 | 80 |
| 16.92 | 16 |
| 17.41 | 60 |
| 19.19 | 22 |
| 20.14 | 24 |
| 21.32 | 20 |
| 22.40 | 43 |
| 24.53 | 11 |
| 24.97 | 83 |
| 24.78 | 20 |
| 24.93 | 100 |

TABLE 4C
Condensed peak list #2 for Compound (I) freebase Pattern D

| 2-theta ± 0.2 (deg) | Relative intensity |
|---|---|
| 8.14 | 18 |
| 11.17 | 87 |
| 11.63 | 42 |
| 12.46 | 37 |
| 15.09 | 80 |
| 17.41 | 60 |
| 20.14 | 24 |
| 22.40 | 43 |
| 24.97 | 83 |
| 24.93 | 100 |

TABLE 4D
Condensed peak list #3 for Compound (1) freebase Pattern D

| 2-theta ± 0.2 (deg) | Relative intensity |
|---|---|
| 11.17 | 87 |
| 11.63 | 42 |
| 15.09 | 80 |
| 24.97 | 83 |
| 24.93 | 100 |

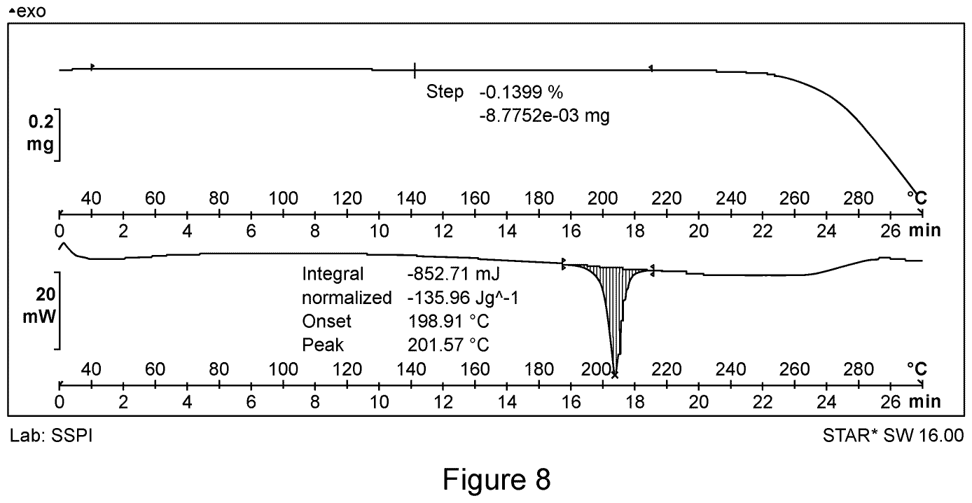

In some embodiments, crystalline solid Form D is also characterized by a DSC thermogram having a broad endotherm with an onset peak at 198.9±2° C. In some embodiments, crystalline solid Form D is also characterized by a DSC thermogram substantially similar to FIG. 8.

In one aspect, provided herein is a fifth crystalline solid form of the free base of Compound (I) designated as crystalline solid Form E. In some embodiments, crystalline solid Form E is a hydrate.

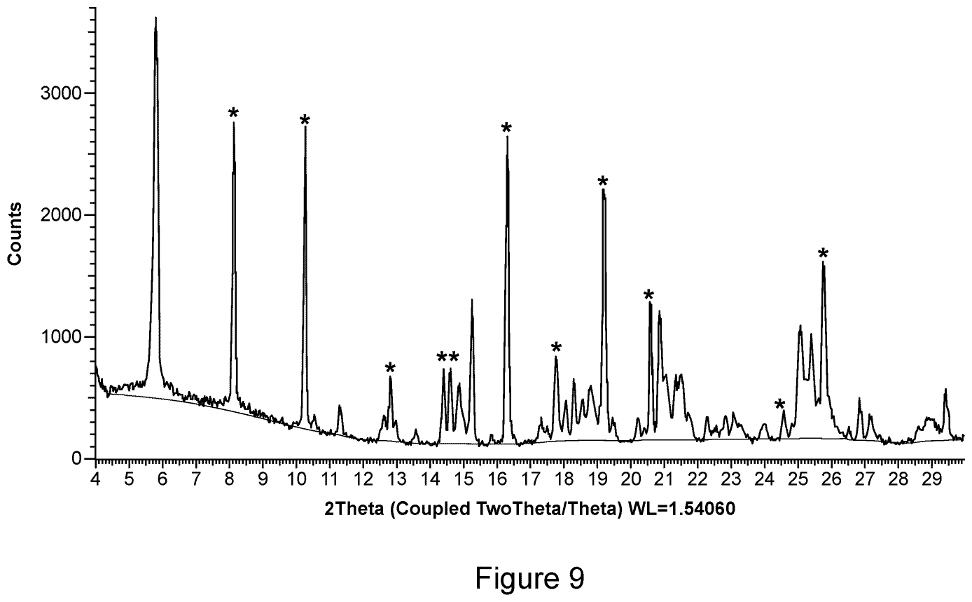

In some embodiments, crystalline solid Form E is a mixture with crystalline solid Forms A and B and is characterized by an XRPD pattern substantially similar to FIG. 9.

Tables 5A-5D list XRPD (2-theta) peaks obtained from samples of crystalline solid Form E of the free base of Compound (I). In some embodiments, crystalline solid Form E is characterized by a XRPD pattern comprising at least three, four, five, six, seven, eight, nine, ten, eleven, twelve, thirteen, fourteen, or fifteen peaks (up to the maximum number of peaks in each table) selected from Table 5A, 5B, 5C, or 5D.

TABLE 5A
Peak list for Compound (I) freebase Pattern E

| 2-theta ± 0.2 (deg) | Relative intensity¹ |
|---|---|
| 8.14 | 75 |
| 10.27 | 78 |
| 12.81 | 17 |
| 12.97 | 5 |
| 14.40 | 20 |
| 14.60 | 19 |
| 16.32 | 80 |
| 17.32 | 6 |
| 18.31 | 16 |
| 19.21 | 65 |
| 20.22 | 6 |
| 20.60 | 36 |
| 21.73 | 7 |
| 22.29 | 6 |
| 25.77 | 46 |
| 26.85 | 10 |
| 27.17 | 6 |

¹Relative intensities < 5 or associated with Form A or B not included

TABLE 5B
Condensed peak list #1 for Compound (I) freebase Pattern E

| 2-theta ± 0.2 (deg) | Relative intensity |
|---|---|
| 8.14 | 75 |
| 10.27 | 78 |
| 12.81 | 17 |
| 14.40 | 20 |
| 14.60 | 19 |
| 16.32 | 80 |
| 17.32 | 6 |
| 18.31 | 16 |
| 19.21 | 65 |
| 20.22 | 6 |
| 20.60 | 36 |
| 21.73 | 7 |
| 22.29 | 6 |
| 25.77 | 46 |
| 26.85 | 10 |

TABLE 5C
Condensed peak list #2 for Compound (I) freebase Pattern E

| 2-theta ± 0.2 (deg) | Relative intensity |
|---|---|
| 8.14 | 75 |
| 10.27 | 78 |
| 12.81 | 17 |
| 14.40 | 20 |
| 14.60 | 19 |
| 16.32 | 80 |
| 18.31 | 16 |
| 19.21 | 65 |
| 20.60 | 36 |
| 25.77 | 46 |

TABLE 5D
Condensed peak list #3 for Compound (I) freebase Pattern E

| 2-theta ± 0.2 (deg) | Relative intensity |
|---|---|
| 8.14 | 75 |
| 10.27 | 78 |
| 16.32 | 80 |
| 19.21 | 65 |
| 20.60 | 36 |

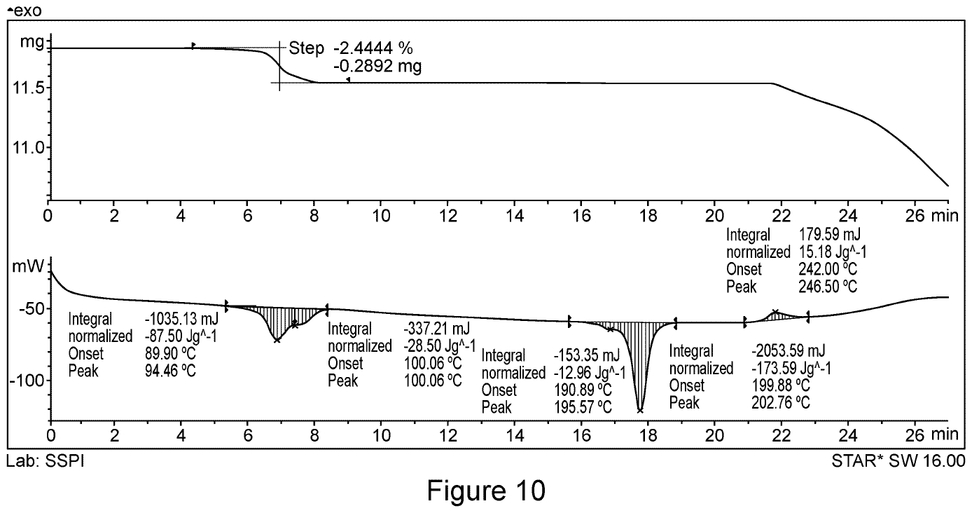

In some embodiments, crystalline solid Form E is also characterized by a DSC thermogram comprising two broad endotherms with onset peaks at 89.9±2° C. and 199.9±2° C. In some embodiments, crystalline solid Form E is also characterized by a DSC thermogram comprising two broad endotherms with onset peaks at 89.9±2° C. and 100.6±2° C., and optionally 190.9±2° C. In some embodiments, crystalline solid Form E is also characterized by a DSC thermogram having four broad endotherms with onset peaks at 89.9±2° C., 100.1±2° C., 190.9±2° C., and 199.9±2° C. and a broad exotherm with an onset peak at 242.0±2° C. In some embodiments, crystalline solid Form E is also characterized by a DSC thermogram substantially similar to FIG. 10.

In one aspect, provided herein is a sixth crystalline solid form of the free base of Compound (I) designated as crystalline solid Form F. In some embodiments, crystalline solid Form F is a hydrate.

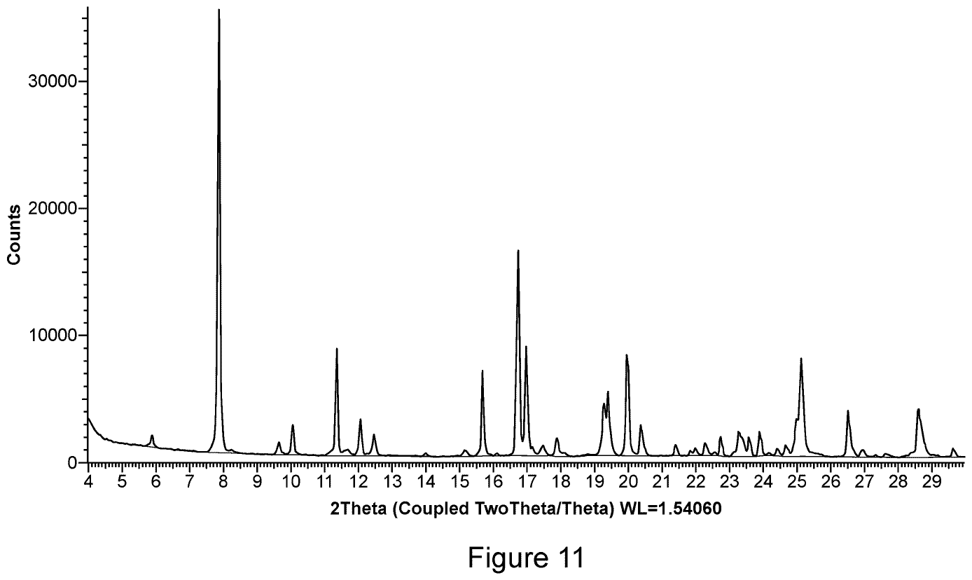

In some embodiments, crystalline Form F is characterized by an XRPD pattern substantially similar to FIG. 11.

Tables 6A-6D list XRPD (2-theta) peaks obtained from samples of crystalline solid Form F of the free base of Compound (I). In some embodiments, the crystalline solid Form F is characterized by a XRPD pattern comprising at least three, four, five, six, seven, eight, nine, ten, eleven, twelve, thirteen, fourteen, or fifteen peaks (up to the maximum number of peaks in each table) selected from Tables 6A, 6B, 6C, or 6D.

TABLE 6A
Peak list for Compound (I) freebase Pattern F

| 2-theta ± 0.2 (deg) | Relative intensity¹ |
|---|---|
| 7.86 | 100 |
| 10.05 | 7 |
| 11.35 | 24 |
| 12.06 | 8 |
| 12.46 | 5 |
| 15.68 | 19 |
| 16.73 | 47 |
| 16.98 | 25 |
| 17.88 | 4 |
| 19.28 | 12 |
| 19.40 | 15 |
| 19.97 | 23 |
| 20.39 | 7 |
| 22.74 | 4 |
| 23.30 | 5 |
| 23.59 | 4 |
| 23.91 | 5 |
| 25.00 | 9 |
| 25.13 | 22 |
| 26.52 | 10 |
| 28.61 | 11 |

¹Relative intensities < 4 not included

TABLE 6B
Condensed peak list #1 for Compound (I) freebase Pattern F

| 2-theta ± 0.2 (deg) | Relative intensity |
|---|---|
| 7.86 | 100 |
| 10.05 | 7 |
| 11.35 | 24 |
| 12.06 | 8 |
| 15.68 | 19 |
| 16.73 | 47 |
| 16.98 | 25 |
| 19.28 | 12 |
| 19.40 | 15 |
| 19.97 | 23 |
| 20.39 | 7 |
| 25.00 | 9 |
| 25.13 | 22 |
| 26.52 | 10 |
| 28.61 | 11 |

TABLE 6C
Condensed peak list #2 for Compound (I) freebase Pattern F

| 2-theta ± 0.2 (deg) | Relative intensity |
|---|---|
| 7.86 | 100 |
| 10.05 | 7 |
| 11.35 | 24 |
| 15.68 | 19 |
| 16.73 | 47 |
| 16.98 | 25 |
| 19.28 | 12 |
| 19.97 | 23 |
| 25.00 | 9 |
| 25.13 | 22 |

TABLE 6D
Condensed peak list #3 for Compound (I) freebase Pattern F

| 2-theta ± 0.2 (deg) | Relative intensity |
|---|---|
| 7.86 | 100 |
| 15.68 | 19 |
| 16.73 | 47 |
| 16.98 | 25 |
| 19.97 | 23 |

In another aspect, provided herein is an amorphous form of the free base of Compound (I) represented by the following structural formula:

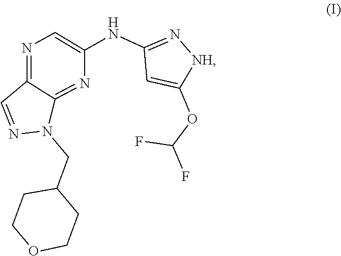 [SMILES: FC(F)Oc1cc(Nc2cnc3cnn(CC4CCOCC4)c3n2)n[nH]1]

In one aspect, provided herein is a method of purifying Compound (I) represented by the following structural formula:

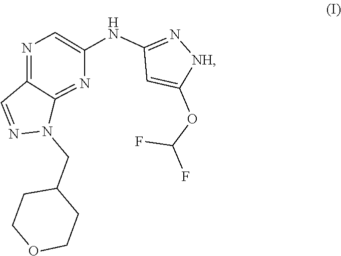 [SMILES: FC(F)Oc1cc(Nc2cnc3cnn(CC4CCOCC4)c3n2)n[nH]1]

comprising recrystallizing the Compound (I) in a solvent mixture to yield a crystalline solid form of Compound (I).

In some embodiments, a crystalline solid form disclosed herein is obtained by a process comprising one or both of the following steps:
a. dissolving the solid form in a solvent mixture to form a solution; and
b. cooling crystallization in the solvent mixture.

In some embodiments, the solvent mixture is selected from a solvent mixture comprising acetone, diethyl ether, ethanol, ethyl acetate, n-heptane, isopropyl alcohol, methanol, and trifluoroethanol.

In some embodiments, a free base crystalline solid Form A is obtained by a process comprising recrystallization of Compound (I) in a solvent mixture comprising methanol, water, and, optionally, ethanol.

In some embodiments, a free base crystalline solid Form A is obtained by a process further comprising one or both of the following steps:
a. dissolving the Compound (I) in methanol at an elevated temperature to form a solution; and
b. evaporative concentration and cooling crystallization in a solvent mixture comprising methanol, ethanol, and water.

In some embodiments, a free base crystalline solid Form A is obtained by a process comprising recrystallization of Compound (I) in a solvent mixture comprising acetone and n-heptane.

In some embodiments, a free base crystalline solid Form A is obtained by a process further comprising one or both of the following steps:
a. dissolving Compound (I) in acetone at an elevated temperature to form a solution; and
b. evaporative concentration and cooling crystallization in a solvent mixture comprising acetone and n-heptane.

In some embodiments, a free base crystalline solid Form B is obtained by a process comprising one or both of the following steps:
a. dissolving Compound (I) in a solvent mixture at an elevated temperature to form a solution; and
b. evaporating the solvent mixture.

In some embodiments, the solvent mixture is selected from a solvent mixture comprising acetone, acetonitrile, ethanol, ethyl acetate, isopropyl acetate, isopropyl alcohol, and 2-methyltetrahydrofuran.

In some embodiments, a free base crystalline solid Form B is obtained by a process comprising one or both of the following steps:
a. dissolving Compound (I) in acetone at an elevated temperature to form a solution; and
b. evaporating the acetone.

In some embodiments, free base crystalline solid Form B is obtained by a process comprising heating Compound (I) at an elevated temperature. In some embodiments, free base crystalline solid Form A, Form C, Form D, or Form E is heated at an elevated temperature to provide free base crystalline solid Form B. In some embodiments, the elevated temperature is at least about 150° C. In some embodiments, the elevated temperature is about 150° C., about 187° C., or about 195° C. In some embodiments, free base crystalline solid Form D or Form F is dried overnight at about 50° C. to provide free base crystalline solid Form B.

In some embodiments, a free base crystalline solid Form C is obtained by a process comprising one or both of the following steps:
a. dissolving Compound (I) in a solvent mixture comprising acetonitrile and water; and
b. lyophilization.

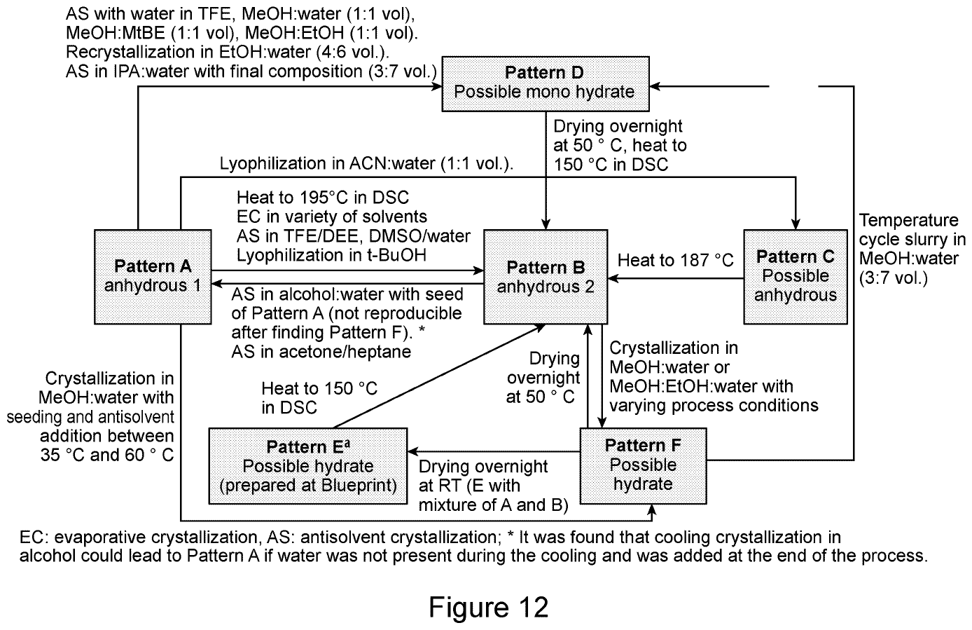

In some embodiments, a process for converting between different crystalline solid forms of the free base of Compound (I) is shown in the schematic of FIG. 12.

### Compound (I) Solid Salt Forms

In another aspect, provided herein is a crystalline solid form of a pharmaceutically acceptable salt of Compound (I) represented by the following structural formula:

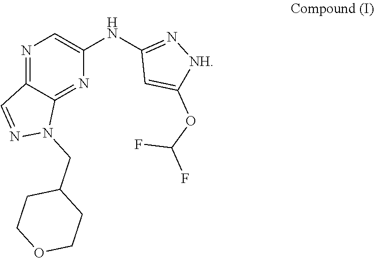 [SMILES: FC(F)Oc1cc(Nc2cnc3cnn(CC4CCOCC4)c3n2)n[nH]1]

The term “pharmaceutically acceptable salt” refers to a pharmaceutical salt that is, within the scope of sound medical judgment, suitable for use in contact with the tissues of humans and lower animals without undue toxicity, irritation, and allergic response, and is commensurate with a reasonable benefit/risk ratio. Pharmaceutically acceptable salts are well known in the art. For example, S. M. Berge et al. describes pharmacologically acceptable salts in J. Pharm. Sci., 1977, 66, 1-19.

In some embodiments, provided herein is a crystalline solid form of a pharmaceutically acceptable salt of Compound (I) represented by the following structural formula:

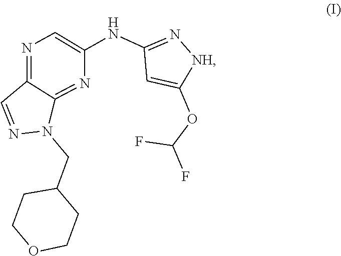 [SMILES: FC(F)Oc1cc(Nc2cnc3cnn(CC4CCOCC4)c3n2)n[nH]1]

wherein the salt is selected from the group consisting of a methanesulfonate salt, an ethanesulfonate salt, a benzenesulfonate salt, a tosylate salt, a naphthalene-2-sulfonate salt, a glycine salt, a succinate salt, a citrate salt, a tartrate salt, an adipate salt, an aspartate salt, a histidine salt, a vanillin salt, a phosphate salt, a hydrochloride salt, hydrobromide salt, a nitrate salt, and a sulfate salt. In some embodiments, the crystalline solid form comprises a pharmaceutically acceptable salt of Compound (I) selected from a salt of methanesulfonic acid, ethanesulfonic acid, benzenesulfonic acid, toluene-4-sulfonic acid, naphthalene-2-sulfonic acid, hydrochloric acid, hydrobromic acid, nitric acid, and sulfuric acid. In some embodiments, the crystalline solid form is a salt of methanesulfonic acid or a methanesulfonate salt. In some embodiments, the crystalline solid form is a salt of ethanesulfonic acid or an ethanesulfonate salt. In some embodiments, the crystalline solid form is a salt of benzensulfonic acid or a besylate salt. In some embodiments, the crystalline solid form is a salt of toluene-4-sulfonic acid or a tosylate salt. In some embodiments, the crystalline solid form is a salt of naphthalene-2-sulfonic acid or a naphthalene-2-sulfonate salt. In some embodiments, the crystalline solid form is a salt of succinic acid or a succinate salt. In some embodiments, the crystalline solid form is a salt of citric acid or a citrate salt. In some embodiments, the crystalline solid form is a salt of succinic acid or a succinate salt. In some embodiments, the crystalline solid form is a salt of tartaric acid or a tartrate salt. In some embodiments, the crystalline solid form is a salt of adipic acid or an adipate salt. In some embodiments, the crystalline solid form is a salt of aspartic acid or an aspartate salt. In some embodiments, the crystalline solid form is a salt of phosphoric acid or a phosphate salt. In some embodiments, the crystalline solid form is a salt of hydrochloric acid or a hydrochloride salt. In some embodiments, the crystalline solid form is a salt of hydrobromic acid or a hydrobromide salt. In some embodiments, the crystalline solid form is a salt of nitric acid or a nitrate salt. In some embodiments, the crystalline solid form is a salt of sulfuric acid or a sulfate salt. In some embodiments, the crystalline solid form is a salt of vanillin. In some embodiments, the crystalline solid form is a salt of histidine. In some embodiments, the crystalline solid form is a salt of glycine.

In some embodiments, the crystalline solid form is of a tosylate salt.

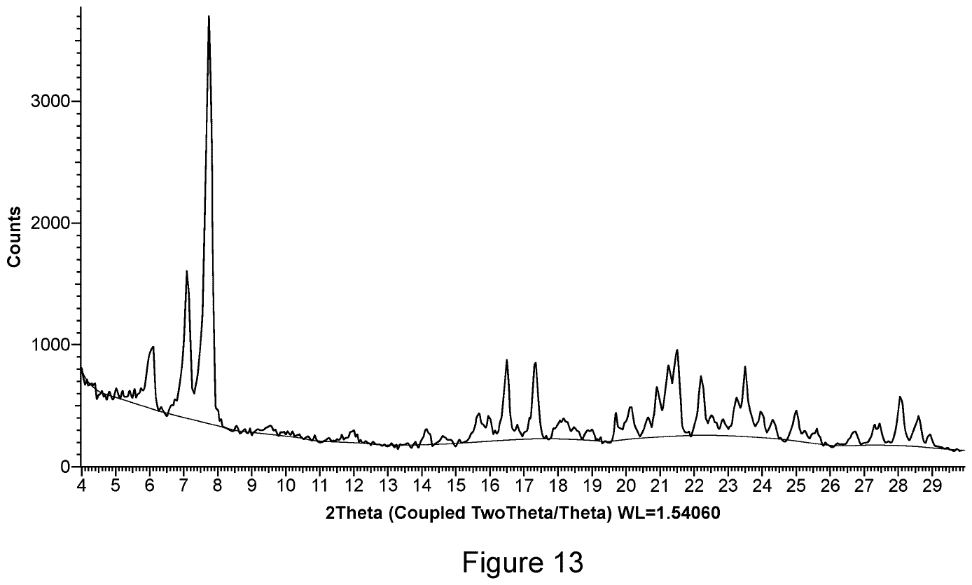

In one aspect, disclosed herein is crystalline Compound (I) tosylate salt Form 6-B. In one embodiment, crystalline Compound (I) tosylate salt Form 6-B is characterized by a XRPD pattern substantially similar to FIG. 13. In one embodiment, crystalline Compound (I) tosylate salt Form 6-B is characterized by a XRPD pattern comprising at least three, four, five, six, seven, eight, nine, ten, eleven, twelve, thirteen, fourteen, or fifteen peaks (up to the maximum number of peaks in each table) selected from Table 7A, 7B, 7C, or 7D.

TABLE 7A
Peak list for Compound (I) tosylate Pattern 6-B

| 2-theta ± 0.2 (deg) | Relative intensity¹ |
|---|---|
| 6.06 | 15 |
| 7.10 | 37 |
| 7.76 | 100 |
| 9.53 | 2 |
| 11.98 | 3 |
| 14.14 | 4 |
| 14.63 | 2 |
| 15.67 | 7 |
| 15.96 | 6 |
| 16.49 | 19 |
| 17.33 | 19 |
| 18.12 | 4 |
| 18.14 | 5 |
| 18.96 | 2 |
| 19.72 | 6 |
| 20.13 | 8 |
| 20.65 | 5 |
| 20.92 | 12 |
| 21.26 | 17 |
| 21.48 | 20 |
| 22.21 | 15 |
| 22.51 | 5 |
| 22.85 | 4 |
| 23.26 | 9 |
| 23.50 | 17 |
| 23.97 | 6 |
| 24.31 | 5 |
| 24.99 | 7 |
| 25.58 | 3 |
| 26.69 | 3 |
| 27.43 | 5 |
| 28.06 | 12 |
| 28.59 | 7 |
| 28.92 | 3 |

¹Relative intensities < 2 not included

TABLE 7B
Condensed peak list #1 for Compound (I) tosylate Pattern 6-B

| 2-theta ± 0.2 (deg) | Relative intensity |
|---|---|
| 6.06 | 15 |
| 7.10 | 37 |
| 7.76 | 100 |
| 16.49 | 19 |
| 17.33 | 19 |
| 20.13 | 8 |
| 20.92 | 12 |
| 21.26 | 17 |
| 21.48 | 20 |
| 22.21 | 15 |
| 23.26 | 9 |
| 23.50 | 17 |
| 24.99 | 7 |
| 28.06 | 12 |
| 28.59 | 7 |

TABLE 7C
Condensed peak list #2 for Compound (I) tosylate Pattern 6-B

| 2-theta ± 0.2 (deg) | Relative intensity |
|---|---|
| 6.06 | 15 |
| 7.10 | 37 |
| 7.76 | 100 |
| 16.49 | 19 |
| 17.33 | 19 |
| 21.26 | 17 |
| 21.48 | 20 |
| 22.21 | 15 |
| 23.50 | 17 |
| 28.06 | 12 |

TABLE 7D
Condensed peak list #3 for Compound (I) tosylate Pattern 6-B

| 2-theta ± 0.2 (deg) | Relative intensity |
|---|---|
| 7.10 | 37 |
| 7.76 | 100 |
| 16.49 | 19 |
| 17.33 | 19 |
| 23.50 | 17 |

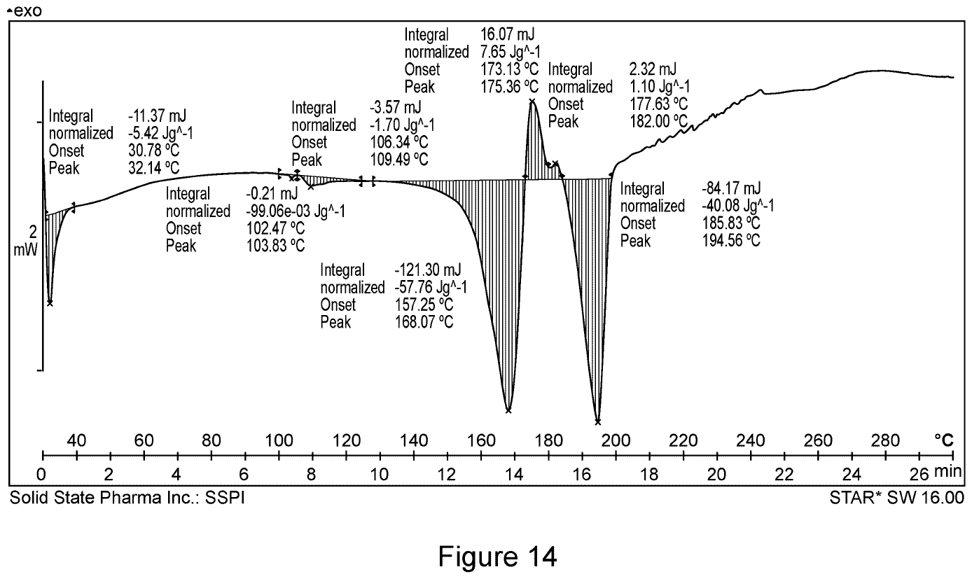

In some embodiments, the crystalline tosylate salt Form 6-B is also characterized by a DSC thermogram comprising two broad endotherms with onset peaks at 157.3±2° C. and 185.8±2° C. In one embodiment, the crystalline Compound (I) tosylate salt Form 6-B has a DSC having five broad endotherms with onset peaks at 30.8±2° C., 102.5±2° C., 106.3±2° C., 157.3±2° C., and 185.8±2° C. and two exotherms with onset peaks at 173.1±2° C. and 177.6±2° C. In one embodiment, crystalline Compound (I) tosylate salt Form 6-B has a DSC that is substantially similar to the DSC shown in FIG. 14.

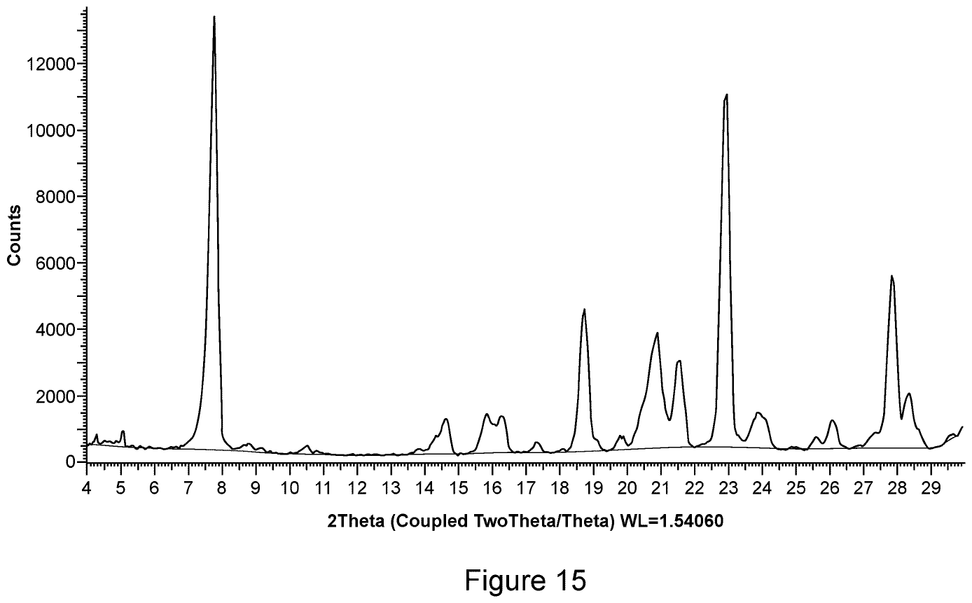

In one aspect, disclosed herein is crystalline Compound (I) tosylate salt Form 6-C. In some embodiments, the crystalline solid form is referred to as crystalline tosylate salt Form 6-C and is characterized by one or both of the following: a) an XRPD pattern substantially similar to FIG. 15; and b) a DSC having an endotherm with an onset peak at 165.5±2° C. In one embodiment, crystalline Compound (I) tosylate salt form 6-C is characterized by a XRPD pattern substantially similar to FIG. 15. In some embodiments, crystalline Compound (I) tosylate salt Form 6-C is characterized by an XRPD pattern comprising at least three, four, five, six, seven, eight, nine, ten, eleven, twelve, thirteen, fourteen, or fifteen peaks (up to the maximum number of peaks in each table) selected from Tables 8A, 8B, 8C, or 8D.

TABLE 8A
Peak list for Compound (I) tosylate Pattern 6-C

| 2-theta ± 0.2 (deg) | Relative intensity¹ |
|---|---|
| 5.08 | 4 |
| 7.79 | 100 |
| 8.79 | 2 |
| 10.53 | 2 |
| 14.63 | 8 |
| 15.86 | 9 |
| 16.31 | 8 |
| 17.34 | 2 |
| 18.74 | 33 |
| 19.85 | 3 |
| 20.88 | 27 |
| 21.56 | 20 |
| 22.93 | 82 |
| 23.90 | 8 |
| 25.62 | 3 |
| 26.10 | 7 |
| 27.39 | 3 |
| 27.86 | 40 |
| 28.34 | 13 |

¹Relative intensities < 2 not included

TABLE 8B
Condensed peak list #1 for Compound (I) tosylate Pattern 6-C

| 2-theta ± 0.2 (deg) | Relative intensity |
|---|---|
| 7.79 | 100 |
| 14.63 | 8 |
| 15.86 | 9 |
| 16.31 | 8 |
| 18.74 | 33 |
| 19.85 | 3 |
| 20.88 | 27 |
| 21.56 | 20 |
| 22.93 | 82 |
| 23.90 | 8 |
| 25.62 | 3 |
| 26.10 | 7 |
| 27.39 | 3 |
| 27.86 | 40 |
| 28.34 | 13 |

TABLE 8C
Condensed peak list #2 for Compound (I) tosylate Pattern 6-C

| 2-theta ± 0.2 (deg) | Relative intensity |
|---|---|
| 7.79 | 100 |
| 14.63 | 8 |
| 15.86 | 9 |
| 18.74 | 33 |
| 20.88 | 27 |
| 21.56 | 20 |
| 22.93 | 82 |
| 23.90 | 8 |
| 27.86 | 40 |
| 28.34 | 13 |

TABLE 8D
Condensed peak list #3 for Compound (I) tosylate Pattern 6-C

| 2-theta ± 0.2 (deg) | Relative intensity |
|---|---|
| 7.79 | 100 |
| 18.74 | 33 |
| 20.88 | 27 |
| 22.93 | 82 |
| 27.86 | 40 |

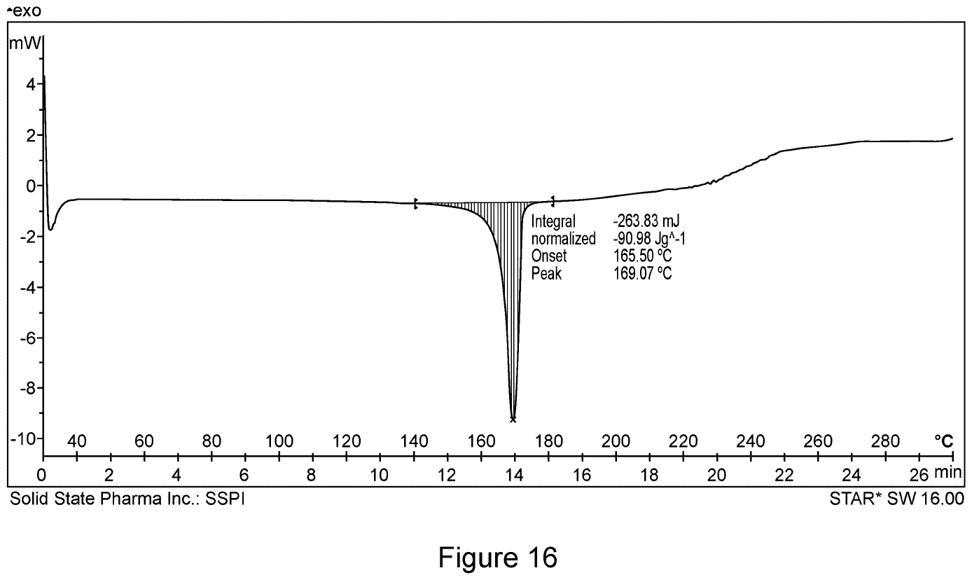

In some embodiments, the crystalline tosylate salt Form 6-C is also characterized by a DSC thermogram comprising an endotherm with an onset peak at 165.5±2° C. In one embodiment, the crystalline Compound (I) tosylate salt Form 6-C has a DSC that is substantially similar to the DSC shown in FIG. 16.

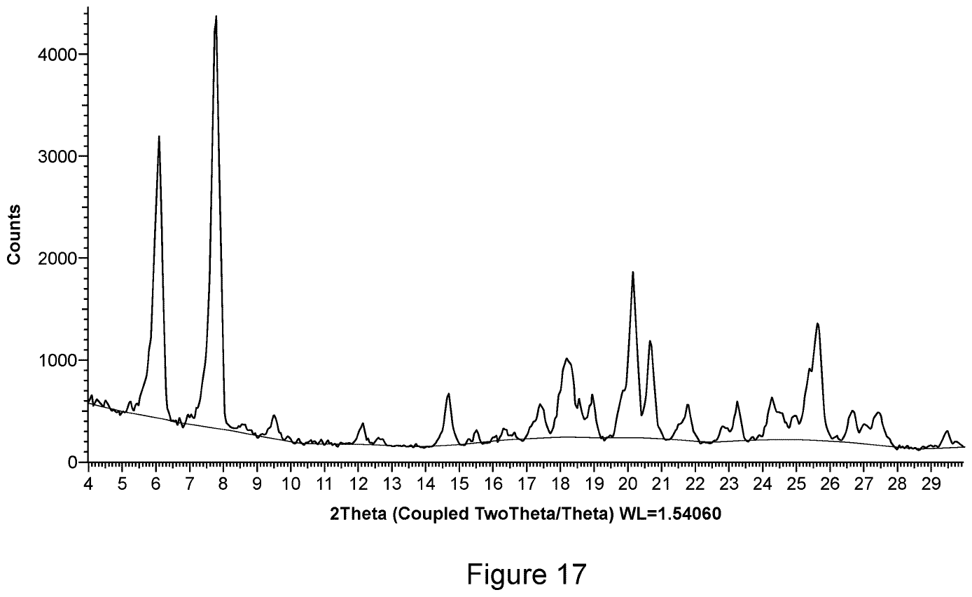

In one aspect, disclosed herein is crystalline Compound (I) tosylate salt Form 6-D. In one embodiment, crystalline Compound (I) tosylate salt form 6-D is characterized by a XRPD pattern substantially similar to FIG. 17. In some embodiments, crystalline Compound (I) tosylate salt Form 6-D is characterized by an XRPD pattern comprising at least three, four, five, six, seven, eight, nine, ten, eleven, twelve, thirteen, fourteen, or fifteen peaks (up to the maximum number of peaks in each table) selected from Tables 9A, 9B, 9C, or 9D.

TABLE 9A
Peak list for Compound (I) tosylate Pattern 6-D

| 2-theta ± 0.2 (deg) | Relative intensity¹ |
|---|---|
| 6.10 | 69 |
| 7.78 | 100 |
| 8.59 | 2 |
| 9.51 | 6 |
| 12.13 | 5 |
| 14.67 | 12 |
| 15.51 | 3 |
| 16.37 | 3 |
| 17.42 | 8 |
| 18.21 | 19 |
| 18.52 | 8 |
| 18.94 | 10 |
| 20.15 | 40 |
| 20.66 | 24 |
| 21.79 | 9 |
| 22.86 | 4 |
| 23.25 | 10 |
| 24.29 | 10 |
| 24.50 | 7 |
| 24.93 | 6 |
| 25.44 | 17 |
| 25.63 | 28 |
| 26.66 | 8 |
| 27.04 | 5 |
| 27.43 | 8 |
| 29.45 | 4 |

¹Relative intensities < 2 not included

TABLE 9B
Condensed peak list #1 for Compound (I) tosylate Pattern 6-D

| 2-theta ± 0.2 (deg) | Relative intensity |
|---|---|
| 6.10 | 69 |
| 7.78 | 100 |
| 14.67 | 12 |
| 18.21 | 19 |
| 18.52 | 8 |
| 18.94 | 10 |
| 20.15 | 40 |
| 20.66 | 24 |
| 21.79 | 9 |
| 23.25 | 10 |
| 24.29 | 10 |
| 25.44 | 17 |
| 25.63 | 28 |
| 26.66 | 8 |
| 27.43 | 8 |

TABLE 9C
Condensed peak list #2 for Compound (I) tosylate Pattern 6-D

| 2-theta ± 0.2 (deg) | Relative intensity |
|---|---|
| 6.10 | 69 |
| 7.78 | 100 |
| 14.67 | 12 |
| 20.15 | 40 |
| 20.66 | 24 |
| 21.79 | 9 |
| 23.25 | 10 |
| 24.29 | 10 |
| 25.44 | 17 |
| 25.63 | 28 |

TABLE 9D
Condensed peak list #3 for Compound (I) tosylate Pattern 6-D

| 2-theta ± 0.2 (deg) | Relative intensity |
|---|---|
| 6.10 | 69 |
| 7.78 | 100 |
| 20.15 | 40 |
| 20.66 | 24 |
| 25.63 | 28 |

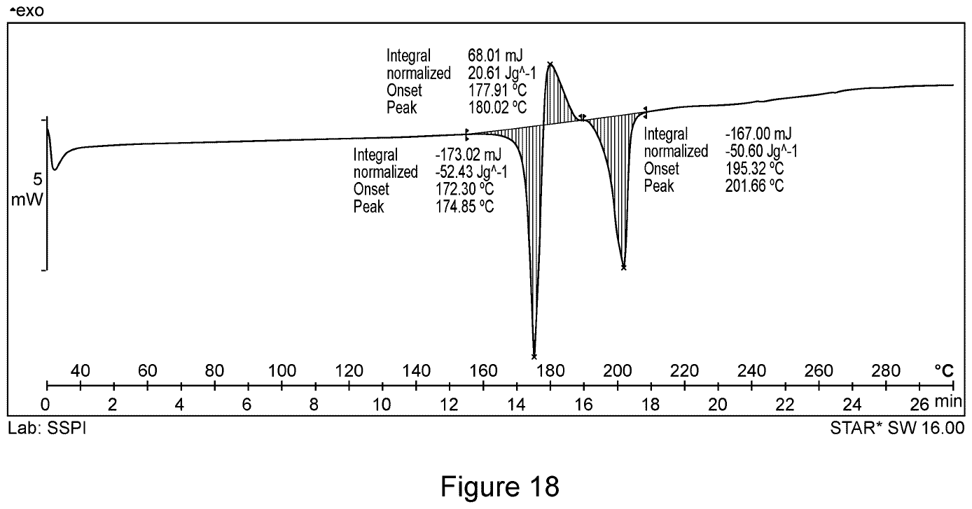

In some embodiments, the crystalline tosylate salt Form 6-D is also characterized by a DSC thermogram comprising two broad endotherms with onset peaks at 172.3±2° C. and 195.3±2° C. In some embodiments, the tosylate salt Form 6-D is also characterized by a DSC thermogram comprising two broad endotherms with onset peaks at 172.3±2° C. and 195.3±2° C. and a broad exotherm with an onset peak at 177.9±2° C. In one embodiment, the crystalline Compound (I) tosylate salt Form 6-D has a DSC/TGA that is substantially similar to the DSC shown in FIG. 18.

In some embodiments, the crystalline solid form is of an ethanesulfonate salt.

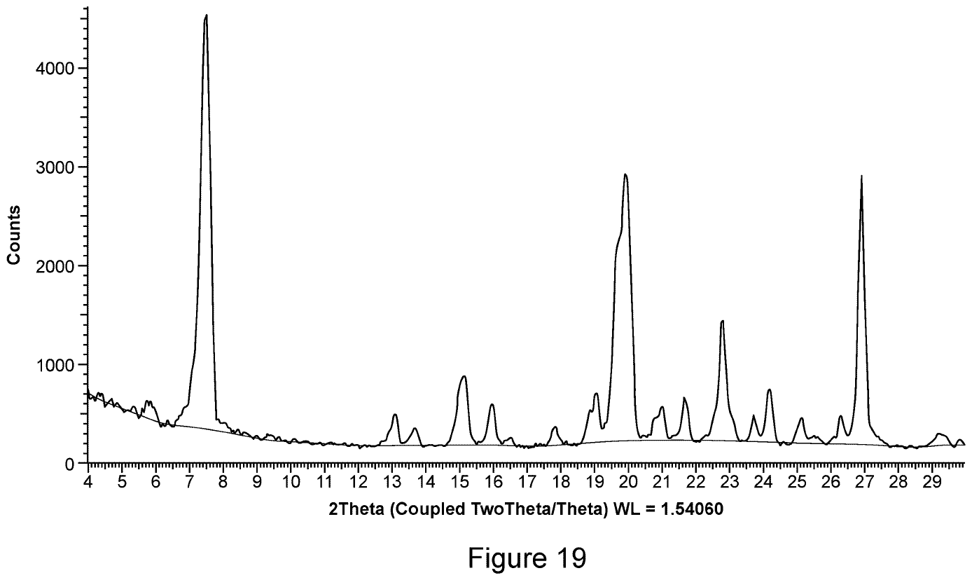

In one aspect, disclosed herein is a crystalline Compound (I) ethanesulfonate salt Form 7-A. In some embodiments, the crystalline solid form is referred to as crystalline ethanesulfonate salt Form 7-A and is characterized by one or both of the following: a) an XRPD pattern substantially similar to FIG. 19; and b) a DSC having a broad endotherm with an onset peak at 183.8±2° C. In one embodiment, crystalline Compound (I) ethanesulfonate salt form 7-A is characterized by a XRPD pattern substantially similar to FIG. 19. In one embodiment, crystalline Compound (I) ethanesulfonate salt Form 7-A is characterized by an XRPD pattern comprising at least three, four, five, six, seven, eight, nine, ten, eleven, twelve, thirteen, fourteen, or fifteen peaks (up to the maximum number of peaks in each table) selected from Tables 10A, 10B, 10C, or 10D.

TABLE 10A
Peak list for Compound (I) ethanesulfonate Pattern 7-A

| 2-theta ± 0.2 (deg) | Relative intensity¹ |
|---|---|
| 5.80 | 3 |
| 7.49 | 100 |
| 13.08 | 8 |
| 13.67 | 4 |
| 15.12 | 16 |
| 15.94 | 10 |
| 16.46 | 2 |
| 17.81 | 4 |
| 19.04 | 12 |
| 19.91 | 64 |
| 20.99 | 8 |
| 21.67 | 10 |
| 22.79 | 29 |
| 23.71 | 6 |
| 24.17 | 13 |
| 25.11 | 6 |
| 26.30 | 7 |
| 26.90 | 64 |
| 29.24 | 3 |

¹Relative intensities < 2 not included

TABLE 10B
Condensed peak list #1 for Compound
(I) ethanesulfonate Pattern 7-A

| 2-theta ± 0.2 (deg) | Relative intensity |
|---|---|
| 7.49 | 100 |
| 13.08 | 8 |
| 15.12 | 16 |
| 15.94 | 10 |
| 17.81 | 4 |
| 19.04 | 12 |
| 19.91 | 64 |
| 20.99 | 8 |
| 21.67 | 10 |
| 22.79 | 29 |
| 23.71 | 6 |
| 24.17 | 13 |
| 25.11 | 6 |
| 26.30 | 7 |
| 26.90 | 64 |

TABLE 10C
Condensed peak list #2 for Compound
(I) ethanesulfonate Pattern 7-A

| 2-theta ± 0.2 (deg) | Relative intensity |
|---|---|
| 7.49 | 100 |
| 13.08 | 8 |
| 15.12 | 16 |
| 15.94 | 10 |
| 19.04 | 12 |
| 19.91 | 64 |
| 21.67 | 10 |
| 22.79 | 29 |
| 24.17 | 13 |
| 26.90 | 64 |

TABLE 10D
Condensed peak list #3 for Compound
(I) ethanesulfonate Pattern 7-A

| 2-theta ± 0.2 (deg) | Relative intensity |
|---|---|
| 7.49 | 100 |
| 15.12 | 16 |
| 19.91 | 64 |
| 22.79 | 29 |
| 26.90 | 64 |

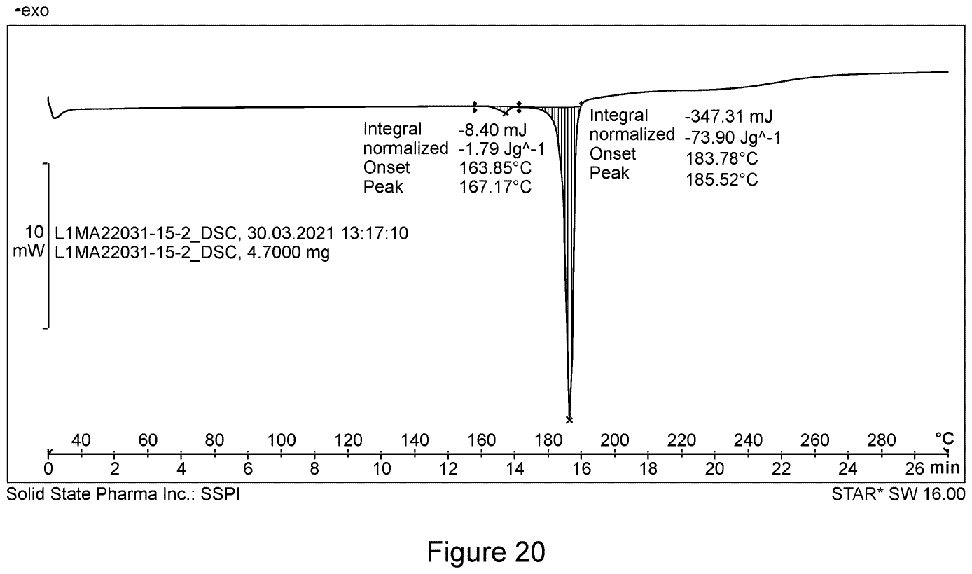

In some embodiments, the crystalline ethanesulfonate salt Form 7-A is also characterized by a DSC thermogram comprising an endotherm with an onset peak at 183.8±2° C. In one embodiment, the crystalline Compound (I) ethanesulfonate salt Form 7-A has a DSC having two broad endotherms with onset peaks at 163.9±2° C. and 183.8±2° C. In one embodiment, crystalline Compound (I) ethanesulfonate salt Form 7-A has a DSC that is substantially similar to the DSC shown in FIG. 20.

In some embodiments, the crystalline solid form is of a naphthalene-2-sulfonate salt.

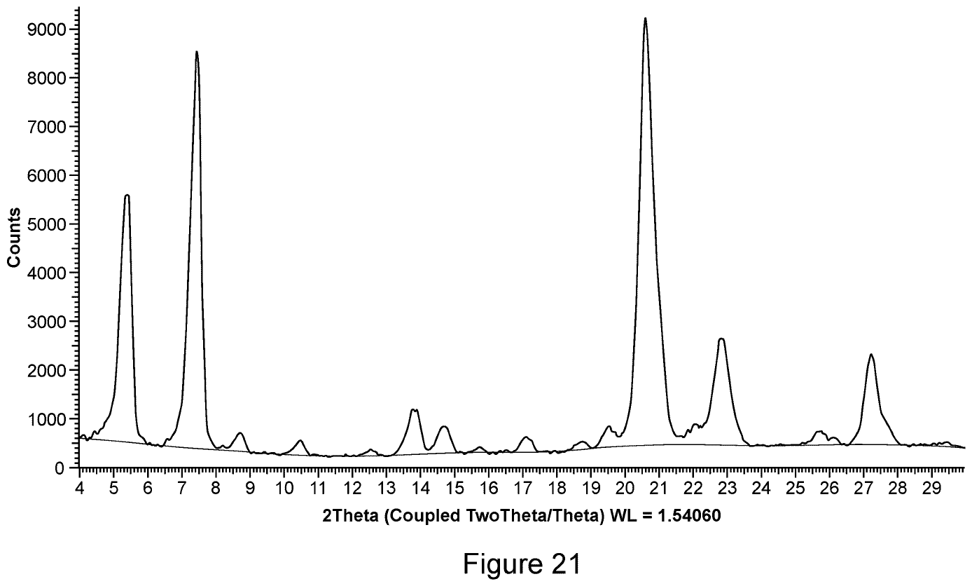

In one aspect, disclosed herein is a crystalline Compound (I) naphthalene-2-sulfonate salt Form 8-A. In one embodiment, crystalline Compound (I) naphthalene-2-sulfonate salt form 8-A is characterized by a XRPD pattern substantially similar to FIG. 21. In some embodiments, the crystalline solid form is referred to as crystalline naphthalene-2-sulfonate salt Forma 8-A and is characterized by one or both of the following: a) an XRPD pattern substantially similar to FIG. 21; and b) a DSC having a broad endotherm with an onset peak at 199.9±2° C. In one embodiment, crystalline Compound (I) naphthalene-2-sulfonate salt Form 8-A is characterized by an XRPD pattern comprising at least three, four, five, six, seven, eight, nine, ten, eleven, twelve, thirteen, fourteen, or fifteen peaks (up to the maximum number of peaks in each table) selected from Tables 11A, 11B, or 11C.

TABLE 11A
Peak list for naphthalene-2-sulfonate Pattern 8-A

| 2-theta ± 0.2 (deg) | Relative intensity¹ |
|---|---|
| 5.40 | 58 |
| 7.46 | 93 |
| 8.71 | 4 |
| 10.47 | 3 |
| 12.55 | 1 |
| 13.83 | 10 |
| 14.69 | 6 |
| 15.72 | 1 |
| 17.10 | 4 |
| 18.74 | 2 |
| 19.51 | 5 |
| 20.59 | 100 |
| 22.83 | 25 |
| 25.76 | 3 |
| 27.21 | 21 |

¹Relative intensities < 1 not included

TABLE 11B
Condensed peak list #1 for naphthalene-2-sulfonate Pattern 8-A

| 2-theta ± 0.2 (deg) | Relative intensity |
|---|---|
| 5.40 | 58 |
| 7.46 | 93 |
| 8.71 | 4 |
| 13.83 | 10 |
| 14.69 | 6 |
| 17.10 | 4 |
| 19.51 | 5 |
| 20.59 | 100 |
| 22.83 | 25 |
| 27.21 | 21 |

TABLE 11C
Condensed peak list #2 for naphthalene-2-sulfonate Pattern 8-A

| 2-theta ± 0.2 (deg) | Relative intensity |
|---|---|
| 5.40 | 58 |
| 7.46 | 93 |
| 20.59 | 100 |
| 22.83 | 25 |
| 27.21 | 21 |

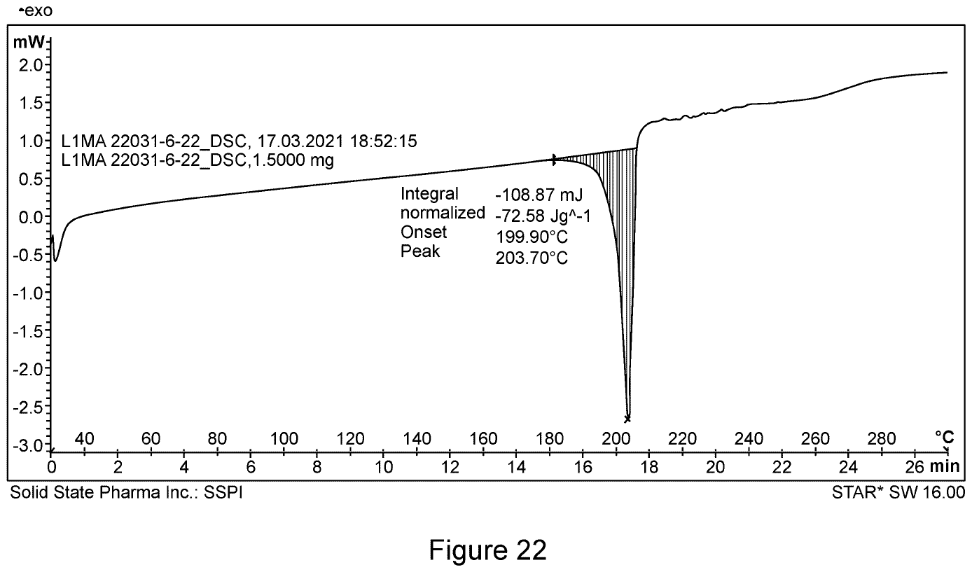

In one embodiment, the crystalline Compound (I) naphthalene-2-sulfonate salt Form 8-A has a DSC having a broad endotherm with an onset peak at 199.9±2° C. In one embodiment, crystalline Compound (I) naphthalene-2-sulfonate salt Form 8-A has a DSC that is substantially similar to the DSC shown in FIG. 22.

In some embodiments, the crystalline solid form is of a hydrochloride salt.

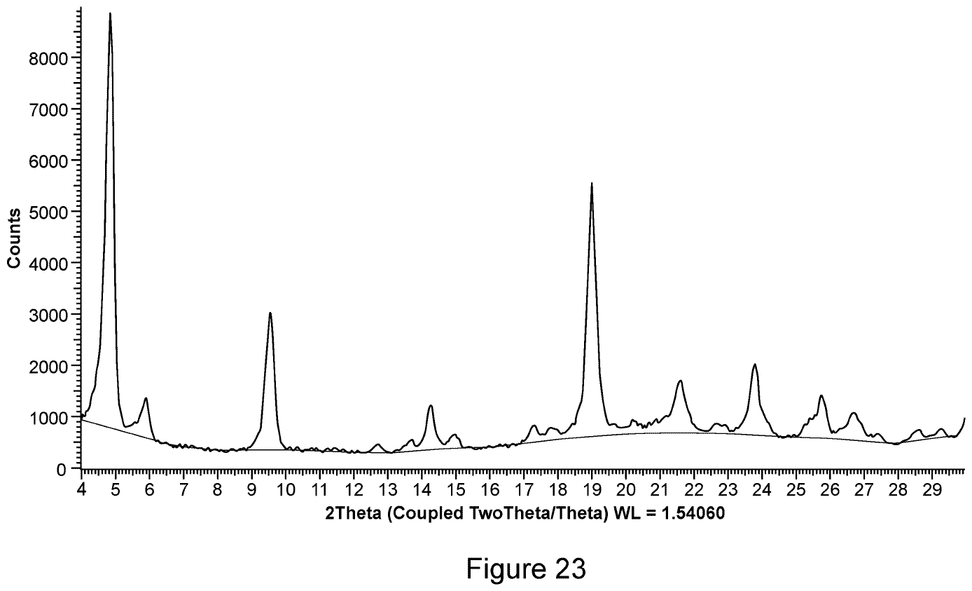

In one aspect, disclosed herein is a crystalline Compound (I) HCl salt Form 1-B. In some embodiments, the crystalline solid form is referred to as crystalline hydrochloride salt Form 1-B and is characterized by one or both of the following: a) an XRPD pattern substantially similar to FIG. 23; and b) a DSC having a broad endotherm with an onset peak at 199.7±2° C. In one embodiment, crystalline Compound (I) HCl salt form 1-B is characterized by a XRPD pattern substantially similar to FIG. 23. In one embodiment, crystalline Compound (I) HCl salt Form 1-B is characterized by an XRPD pattern comprising at least three, four, five, six, seven, eight, nine, ten, eleven, twelve, thirteen, fourteen, or fifteen peaks (up to the maximum number of peaks in each table) selected from Tables 12A, 12B, 12C, or 12D.

TABLE 12A
Peak list for Compound (I) HCl salt Pattern 1-B

| 2-theta ± 0.2 (deg) | Relative intensity¹ |
|---|---|
| 4.85 | 100 |
| 5.89 | 10 |
| 9.55 | 33 |
| 12.70 | 2 |
| 13.68 | 3 |
| 14.26 | 11 |
| 14.95 | 3 |
| 17.30 | 4 |
| 17.89 | 3 |
| 19.00 | 61 |
| 20.31 | 3 |
| 21.59 | 13 |
| 22.67 | 2 |
| 23.79 | 17 |
| 25.76 | 10 |
| 26.71 | 6 |
| 27.40 | 2 |
| 28.59 | 2 |
| 29.26 | 2 |

¹Relative intensities < 2 not included

TABLE 12B
Condensed peak list #1 for Compound (I) HCl salt Pattern 1-B

| 2-theta ± 0.2 (deg) | Relative intensity |
|---|---|
| 4.85 | 100 |
| 5.89 | 10 |
| 9.55 | 33 |
| 13.68 | 3 |
| 14.26 | 11 |
| 14.95 | 3 |
| 17.30 | 4 |
| 17.89 | 3 |
| 19.00 | 61 |
| 20.31 | 3 |
| 21.59 | 13 |
| 22.67 | 2 |
| 23.79 | 17 |
| 25.76 | 10 |
| 26.71 | 6 |

TABLE 12C
Condensed peak list #2 for Compound (I) HCl salt Pattern 1-B

| 2-theta ± 0.2 (deg) | Relative intensity |
|---|---|
| 4.85 | 100 |
| 5.89 | 10 |
| 9.55 | 33 |
| 14.26 | 11 |
| 17.30 | 4 |
| 19.00 | 61 |
| 21.59 | 13 |
| 23.79 | 17 |
| 25.76 | 10 |
| 26.71 | 6 |

TABLE 12D
Condensed peak list #3 for Compound (I) HCl salt Pattern 1-B

| 2-theta ± 0.2 (deg) | Relative intensity |
|---|---|
| 4.85 | 100 |
| 9.55 | 33 |
| 19.00 | 61 |
| 21.59 | 13 |
| 23.79 | 17 |

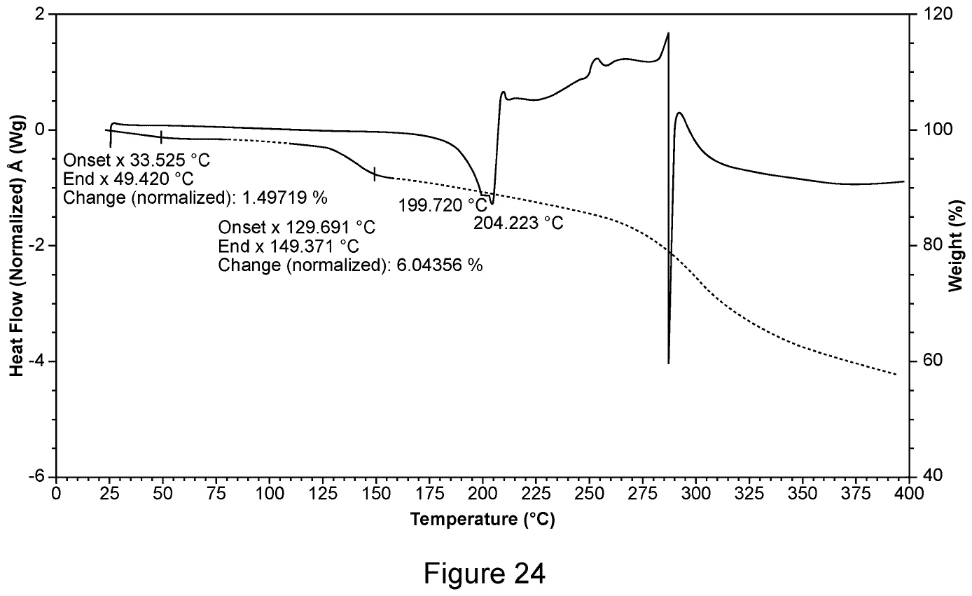

In one embodiment, the crystalline Compound (I) HCl salt Form 1-B has a DSC comprising a broad endotherm with an onset peak at 199.7±2° C. In one embodiment, the crystalline Compound (I) hydrochloride salt Form 1-B has a DSC having two broad endotherms with onset peaks at 199.7±2° C. and 204.2±2° C. In one embodiment, crystalline Compound (I) HCl salt Form 1-B has a DSC that is substantially similar to the TGA/DSC shown in FIG. 24.

In some embodiments, the crystalline solid form is of a hydrobromide salt.

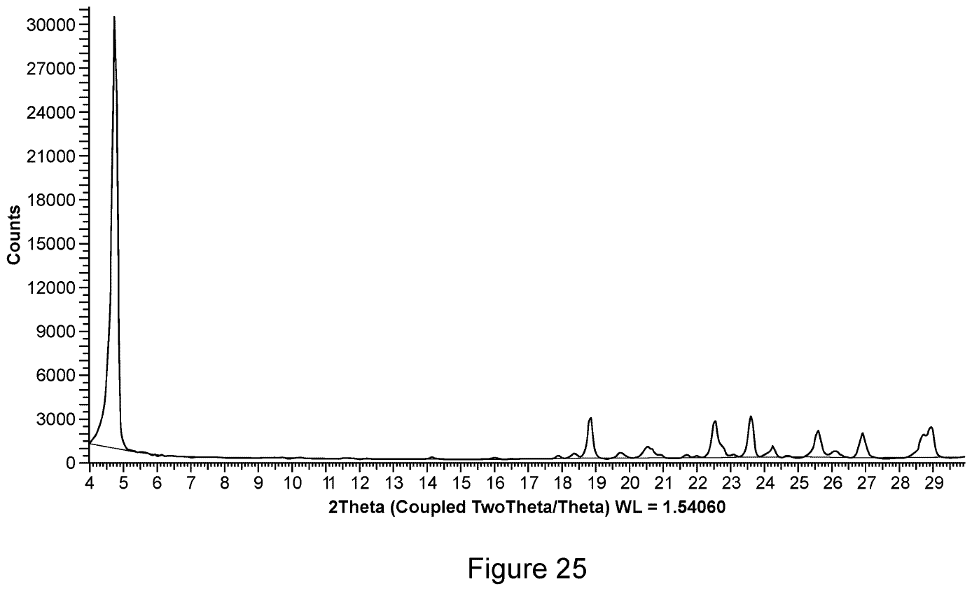

In one aspect, disclosed herein is a crystalline Compound (I) HBr salt Form 2-A. In one embodiment, crystalline Compound (I) HBr salt form 2-A is characterized by a XRPD pattern substantially similar to FIG. 25. In one embodiment, crystalline Compound (I) HBr salt Form 2-A is characterized by an XRPD pattern comprising at least three, four, five, six, seven, eight, nine, ten, eleven, twelve, thirteen, fourteen, or fifteen peaks (up to the maximum number of peaks in each table) selected from Tables 13A, 13B, 13C, or 13D.

TABLE 13A
Peak list for Compound (I) HBr salt Pattern 2-A

| 2-theta ± 0.2 (deg) | Relative intensity¹ |
|---|---|
| 4.75 | 100 |
| 17.89 | 1 |
| 18.36 | 1 |
| 18.84 | 9 |
| 19.74 | 1 |
| 20.53 | 3 |
| 20.91 | 1 |
| 21.69 | 1 |
| 22.53 | 8 |
| 23.07 | 1 |
| 23.60 | 10 |
| 24.24 | 3 |
| 25.59 | 6 |
| 26.08 | 2 |
| 26.91 | 6 |
| 28.73 | 5 |
| 28.92 | 7 |

¹Relative intensities < 1 not included

TABLE 13B
Condensed peak list #1 for Compound (I) HBr salt Pattern 2-A

| 2-theta ± 0.2 (deg) | Relative intensity |
|---|---|
| 4.75 | 100 |
| 17.89 | 1 |
| 18.84 | 9 |
| 19.74 | 1 |
| 20.53 | 3 |
| 21.69 | 1 |
| 22.53 | 8 |
| 23.07 | 1 |
| 23.60 | 10 |
| 24.24 | 3 |
| 25.59 | 6 |
| 26.08 | 2 |
| 26.91 | 6 |
| 28.73 | 5 |
| 28.92 | 7 |

TABLE 13C
Condensed peak list #2 for Compound (I) HBr salt Pattern 2-A

| 2-theta ± 0.2 (deg) | Relative intensity |
|---|---|
| 4.75 | 100 |
| 18.84 | 9 |
| 20.53 | 3 |
| 22.53 | 8 |
| 23.60 | 10 |
| 24.24 | 3 |
| 25.59 | 6 |
| 26.91 | 6 |
| 28.73 | 5 |
| 28.92 | 7 |

TABLE 13D
Condensed peak list #3 for Compound (I) HBr salt Pattern 2-A

| 2-theta ± 0.2 (deg) | Relative intensity |
|---|---|
| 4.75 | 100 |
| 18.84 | 9 |
| 22.53 | 8 |
| 23.60 | 10 |
| 26.91 | 6 |

In some embodiments, the crystalline solid form is of a besylate salt.

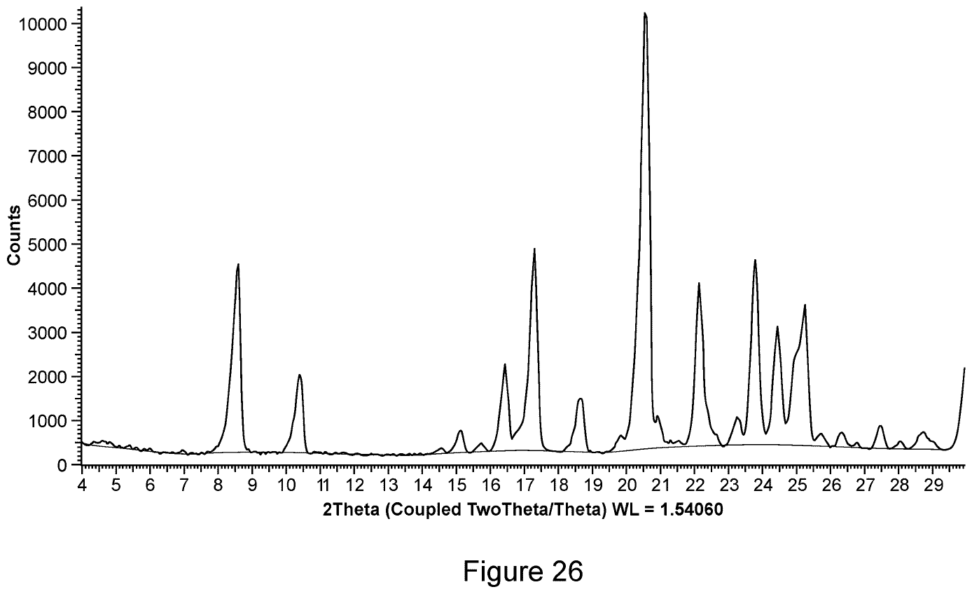

In one aspect, disclosed herein is a crystalline Compound (I) besylate salt Form 3-A. In one embodiment, crystalline Compound (I) besylate salt form 3-A is characterized by a XRPD pattern substantially similar to FIG. 26. In one embodiment, crystalline Compound (I) besylate salt Form 3-A is characterized by an XRPD pattern comprising at least three, four, five, six, seven, eight, nine, ten, eleven, twelve, thirteen, fourteen, or fifteen peaks (up to the maximum number of peaks in each table) selected from Tables 14A, 14B, 14C, or 14D.

TABLE 14A
Peak list for Compound (I) besylate Pattern 3-A

| 2-theta ± 0.2 (deg) | Relative intensity¹ |
|---|---|
| 8.58 | 43 |
| 10.40 | 18 |
| 14.55 | 1 |
| 15.13 | 5 |
| 15.74 | 2 |
| 16.44 | 20 |
| 17.29 | 46 |
| 18.64 | 12 |
| 20.57 | 100 |
| 20.92 | 7 |
| 22.15 | 38 |
| 23.28 | 6 |
| 23.79 | 42 |
| 24.45 | 27 |
| 25.24 | 32 |
| 25.72 | 3 |
| 26.32 | 3 |
| 26.77 | 1 |
| 27.47 | 5 |
| 28.06 | 2 |
| 28.73 | 4 |

¹Relative intensities < 1 not included

TABLE 14B
Condensed peak list #1 for Compound (I) besylate Pattern 3-A

| 2-theta ± 0.2 (deg) | Relative intensity |
|---|---|
| 8.58 | 43 |
| 10.40 | 18 |
| 15.13 | 5 |
| 16.44 | 20 |
| 17.29 | 46 |
| 18.64 | 12 |
| 20.57 | 100 |
| 20.92 | 7 |
| 22.15 | 38 |
| 23.28 | 6 |
| 23.79 | 42 |
| 24.45 | 27 |
| 25.24 | 32 |
| 27.47 | 5 |
| 28.73 | 4 |

TABLE 14C
Condensed peak list #2 for Compound (I) besylate Pattern 3-A

| 2-theta ± 0.2 (deg) | Relative intensity |
|---|---|
| 8.58 | 43 |
| 10.40 | 18 |
| 16.44 | 20 |
| 17.29 | 46 |
| 18.64 | 12 |
| 20.57 | 100 |
| 22.15 | 38 |
| 23.79 | 42 |
| 24.45 | 27 |
| 25.24 | 32 |

TABLE 14D
Condensed peak list #3 for Compound (I) besylate Pattern 3-A

| 2-theta ± 0.2 (deg) | Relative intensity |
|---|---|
| 8.58 | 43 |
| 17.29 | 46 |
| 20.57 | 100 |
| 22.15 | 38 |
| 23.79 | 42 |

In some embodiments, the crystalline solid form is of a mesylate salt.

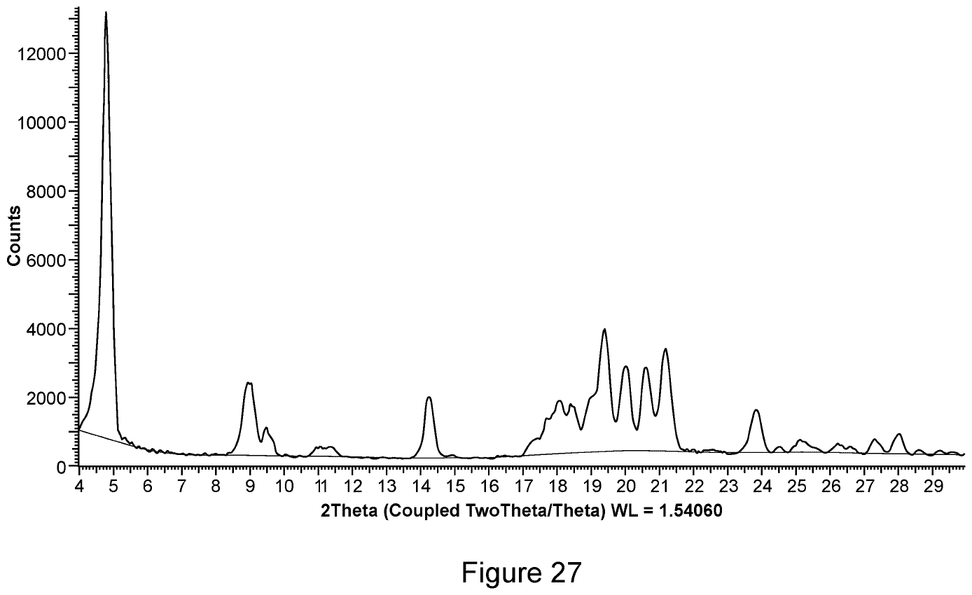

In one aspect, disclosed herein is a crystalline Compound (I) mesylate salt Form 4-A. In one embodiment, crystalline Compound (I) mesylate salt form 4-A is characterized by a XRPD pattern substantially similar to FIG. 27. In one embodiment, crystalline Compound (I) mesylate salt Form 4-A is characterized by an XRPD pattern comprising at least three, four, five, six, seven, eight, nine, ten, eleven, twelve, thirteen, fourteen, or fifteen peaks (up to the maximum number of peaks in each table) selected from Tables 15A, 15B, 15C, or 15D.

TABLE 15A
Peak list for Compound (I) mesylate Pattern 4-A

| 2-theta ± 0.2 (deg) | Relative intensity¹ |
|---|---|
| 4.79 | 100 |
| 9.00 | 17 |
| 9.49 | 7 |
| 11.10 | 2 |
| 11.37 | 2 |
| 14.25 | 15 |
| 17.44 | 4 |
| 17.75 | 8 |
| 18.07 | 12 |
| 18.44 | 11 |
| 19.39 | 29 |
| 20.02 | 20 |
| 20.62 | 20 |
| 21.19 | 24 |
| 23.85 | 10 |
| 25.16 | 3 |
| 26.23 | 2 |
| 26.60 | 2 |
| 27.32 | 3 |
| 28.02 | 5 |

¹Relative intensities <2 not included

TABLE 15B
Condensed peak list #1 for Compound (I) mesylate Pattern 4-A

| 2-theta ± 0.2 (deg) | Relative intensity |
|---|---|
| 4.79 | 100 |
| 9.00 | 17 |
| 9.49 | 7 |
| 14.25 | 15 |
| 17.44 | 4 |
| 17.75 | 8 |
| 18.07 | 12 |
| 18.44 | 11 |
| 19.39 | 29 |
| 20.02 | 20 |
| 20.62 | 20 |
| 21.19 | 24 |
| 23.85 | 10 |
| 25.16 | 3 |
| 28.02 | 5 |

TABLE 15C
Condensed peak list #2 for Compound (I) mesylate Pattern 4-A

| 2-theta ± 0.2 (deg) | Relative intensity |
|---|---|
| 4.79 | 100 |
| 9.00 | 17 |
| 14.25 | 15 |
| 18.07 | 12 |
| 19.39 | 29 |
| 20.02 | 20 |
| 20.62 | 20 |
| 21.19 | 24 |
| 23.85 | 10 |
| 28.02 | 5 |

TABLE 15D
Condensed peak list #3 for Compound (I) mesylate Pattern 4-A

| 2-theta ± 0.2 (deg) | Relative intensity |
|---|---|
| 4.79 | 100 |
| 9.00 | 17 |
| 14.25 | 15 |
| 19.39 | 29 |
| 21.19 | 24 |

In some embodiments, the crystalline solid form is of a sulfate salt.

In one aspect, disclosed herein is a crystalline Compound (I) sulfate salt Form 5-B. In one embodiment, crystalline Compound (I) sulfate salt Form 5-B is characterized by an XRPD pattern an XRPD pattern comprising at least three, four, five, six, seven, eight, nine, ten, eleven, twelve, thirteen, fourteen, or fifteen peaks (up to the maximum number of peaks in each table) selected from Tables 16A, 16B, 16C, or 16D.

TABLE 16A
Peak list for Compound (I) sulfate Pattern 5-B

| 2-theta ± 0.2 (deg) | Relative intensity |
|---|---|
| 4.63 | 100 |
| 5.94 | 6 |
| 9.09 | 1 |
| 10.06 | 1 |
| 10.51 | 1 |
| 12.03 | 1 |
| 14.93 | 1 |
| 16.23 | 1 |
| 17.39 | 1 |
| 18.91 | 1 |
| 20.02 | 3 |
| 20.48 | 5 |
| 21.19 | 2 |
| 21.97 | 12 |
| 23.06 | 1 |
| 23.96 | 2 |
| 24.50 | 2 |
| 25.28 | 2 |
| 26.14 | 1 |
| 26.30 | 1 |
| 26.95 | 2 |
| 27.23 | 2 |
| 28.21 | 4 |
| 28.65 | 1 |

¹Relative intensities <1 not included

TABLE 16B
Condensed peak list #1 for Compound (I) sulfate Pattern 5-B

| 2-theta ± 0.2 (deg) | Relative intensity |
|---|---|
| 4.63 | 100 |
| 5.94 | 6 |
| 18.91 | 1 |
| 20.02 | 3 |
| 20.48 | 5 |
| 21.19 | 2 |
| 21.97 | 12 |
| 23.06 | 1 |
| 23.96 | 2 |
| 24.50 | 2 |
| 25.28 | 2 |
| 26.14 | 1 |
| 26.95 | 2 |
| 27.23 | 2 |
| 28.21 | 4 |

TABLE 16C
Condensed peak list #2 for Compound (I) sulfate Pattern 5-B

| 2-theta ± 0.2 (deg) | Relative intensity |
|---|---|
| 4.63 | 100 |
| 5.94 | 6 |
| 20.02 | 3 |
| 20.48 | 5 |
| 21.19 | 2 |
| 21.97 | 12 |
| 24.50 | 2 |
| 25.28 | 2 |
| 27.23 | 2 |
| 28.21 | 4 |

TABLE 16D
Condensed peak list #3 for Compound (I) sulfate Pattern 5-B

| 2-theta ± 0.2 (deg) | Relative intensity |
|---|---|
| 4.63 | 100 |
| 5.94 | 6 |
| 20.48 | 5 |
| 21.97 | 12 |
| 28.21 | 4 |

### Pharmaceutical Compositions

Pharmaceutical compositions of the disclosure (also referred to herein as the “disclosed pharmaceutical compositions”) comprise one or more pharmaceutically acceptable carrier(s) or diluent(s) and a crystalline solid form of the free base of Compound (I) disclosed herein, the amorphous form of the free base of Compound (I) disclosed herein, or a crystalline solid form of a pharmaceutically acceptable salt of Compound (I) disclosed herein.

“Pharmaceutically acceptable carrier” and “pharmaceutically acceptable diluent” refer to a substance that aids the formulation and/or administration of an active agent to and/or absorption by a subject and can be included in the pharmaceutical compositions of the disclosure without causing a significant adverse toxicological effect on the subject. Non-limiting examples of pharmaceutically acceptable carriers and/or diluents include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, hydroxymethycellulose, fatty acid esters, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with or interfere with the activity of crystalline solid forms of the free base of Compound (I) disclosed herein, the amorphous form of the free base of Compound (I) disclosed herein, or crystalline solid forms of a pharmaceutically acceptable salt of Compound (I) disclosed herein. One of ordinary skill in the art will recognize that other pharmaceutical excipients are suitable for use with the crystalline solid forms of the free base of Compound (I) disclosed herein, the amorphous form of the free base of Compound (I) disclosed herein, or the crystalline solid forms of a pharmaceutically acceptable salt of Compound (I) disclosed herein.

The pharmaceutical compositions of the disclosure optionally include one or more pharmaceutically acceptable carriers and/or diluents therefor, such as lactose, starch, cellulose and dextrose. Other excipients, such as flavoring agents, sweeteners, and preservatives, such as methyl, ethyl, propyl and butyl parabens, can also be included. More complete listings of suitable excipients can be found in the Handbook of Pharmaceutical Excipients (5^{th} Ed., Pharmaceutical Press (2005)). A person skilled in the art would know how to prepare formulations suitable for various types of administration routes. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. The carriers, diluents and/or excipients are “acceptable” in the sense of being compatible with the other ingredients of the pharmaceutical composition and not deleterious to the recipient thereof.

In one aspect, provided herein is a pharmaceutical composition comprising a crystalline solid form of the free base of Compound (I) disclosed herein, the amorphous form of the free base of Compound (I) disclosed herein, or a crystalline solid form of a pharmaceutically acceptable salt of Compound (I) disclosed herein, and a pharmaceutically acceptable carrier.

In one aspect, provided herein is a pharmaceutical composition comprising Compound (I) or a pharmaceutically acceptable salt thereof and a pharmaceutically acceptable carrier, wherein at least 0.1%, 0.5%, 1%, 5%, or more of the Compound (I) or pharmaceutically acceptable salt thereof by weight is present in a crystalline solid form of the free base of Compound (I) disclosed herein, the amorphous form of the free base of Compound (I) disclosed herein, or a crystalline solid form of a pharmaceutically acceptable salt of Compound (I) disclosed herein.

### Methods of Treatment

The solid forms disclosed herein inhibit CDK2 and therefore are useful for treating diseases for which CDK2 is dysregulated, such as cancer. The present disclosure provides a method of inhibiting CDK2 in a subject in need thereof, comprising administering to the subject an effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein.

In some embodiments, the disclosure provides a method of treating a disease or disorder associated with CDK2 in a patient, comprising administering to the patient a therapeutically effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein. In some embodiments, the disease or disorder associated with CDK2 is associated with an amplification of the cyclin E1 (CCNE1) gene and/or overexpression of CCNE1. In some embodiments, the disease or disorder is cancer.

Subjects “in need of inhibiting CDK2” are those having a disease for which a beneficial therapeutic effect can be achieved by inhibiting CDK2, e.g., a slowing in disease progression, alleviation of one or more symptoms associated with the disease or increasing the longevity of the subject in view of the disease.

In some embodiments, the disclosure provides a method of treating a disease/condition/or cancer associated with or modulated by CDK2, wherein the inhibition of CDK2 is of therapeutic benefit, including but not limited to the treatment of cancer in a subject in need thereof. The method comprises administering to the subject an effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein.

In another embodiment, the disclosure provides a method of treating a subject with cancer, comprising administering to the subject an effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein. In another embodiment, the cancer is characterized by amplification and/or overexpression of CCNE1 or CCNE2.

Accordingly, in some embodiments of the methods, the subject or patient has been previously determined to have an amplification of the cyclin E1 (CCNE1) gene and/or an expression level of CCNE1 in a biological sample obtained from the subject or patient that is higher than a control expression level of CCNE1.

In another embodiment, the disclosure provides a method for inhibiting growth of tumor (e.g., cancer) cells in vitro. The method includes contacting the tumor (e.g. cancer) cells in vitro with a crystalline solid form of the free base of Compound (I) disclosed herein, the amorphous form of the free base of Compound (I) disclosed herein, or a crystalline solid form of a pharmaceutically acceptable salt of Compound (I) disclosed herein. In another embodiment, the present disclosure provides a method for inhibiting growth of tumor (e.g., cancer) cells with CCNE1 amplification and/or overexpression in a subject or a patient. The method includes administering to the subject or patient in need thereof a therapeutically effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein.

In another embodiment, the disclosure provides a method of treating a subject with cancer, comprising administering to the subject an effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein in conjunction with other agents or standard cancer treatments, as described below.

As used herein “cancer” refers to an malignant and/or invasive growth or tumor caused by abnormal cell growth. Cancer includes solid tumors named for the type of cells that form them, cancer of blood, bone marrow, or the lymphatic system. Examples of solid tumors include sarcomas and carcinomas. Cancers of the blood include, but are not limited to, leukemia, lymphoma and myeloma. Cancer also includes primary cancer that originates at a specific site in the body, a metastatic cancer that has spread from the place in which it started to other parts of the body, a recurrence from the original primary cancer after remission, and a second primary cancer that is a new primary cancer in a person with a history of previous cancer of a different type from the latter one. In some such embodiments, the cancer is characterized by amplification and/or overexpression of CCNE1 and/or CCNE2.

Cancers to be treated according to the disclosed methods include breast cancer, ovarian cancer, bladder cancer, uterine cancer (e.g., uterine carcinosarcoma), prostate cancer, lung cancer (including NSCLC, SCLC, squamous cell carcinoma (e.g., lung squamous cell carcinoma (LUSC), or adenocarcinoma (e.g., lung adenocarcinoma (LUAD)), esophageal cancer, head and neck cancer, colorectal cancer (e.g., colon cancer, colorectal adenocarcinoma (COADREAD)), kidney cancer (including RCC), liver cancer (including HCC), pancreatic cancer, stomach (i.e., gastric) cancer, urothelial cancer, brain cancers, mesothelioma (MESO), skin cancer (e.g., melanoma), sarcoma, or thyroid cancer, including metastasis (in particular brain metastasis) of all cancers listed. In some embodiments, the cancer is characterized by at overexpression and/or amplification of CCNE1 and/or CCNE2 described herein. In some embodiments of the methods provided herein, the subject is identified as having a cancer characterized by amplification and/or overexpression of CCNE1 and/or CCNE2.

In further embodiments of the methods provided herein, the cancer is breast cancer, ovarian cancer, bladder cancer, uterine cancer, prostate cancer, lung cancer, esophageal cancer, liver cancer, pancreatic cancer or stomach cancer. In some such embodiments, the cancer is characterized by amplification and/or overexpression of CCNE1 and/or CCNE2.

In further embodiments, of the methods provided herein, the cancer is selected from the group consisting of ovarian cancer, endometrial cancer, gastric cancer, esophageal cancer, triple negative breast cancer, and lung adenosarcoma. In some embodiments, the cancer is characterized by CCNE1 overexpression and/or amplification. In some embodiments, the cancer has progressed despite platinum treatment.

In some embodiments, the cancer is platinum-resistant and/or platinum-refractory. In some embodiments, the cancer has progressed despite platinum treatment.

In some embodiments, the disease or disorder associated with CDK2 is an adenocarcinoma, carcinoma, or cystadenocarcinoma.

In other embodiments, the cancer is breast cancer, including, e.g., ER-positive/HR-positive, HER2-negative breast cancer; ER-positive/HR-positive, HER2-positive breast cancer; triple negative breast cancer (TNBC); or inflammatory breast cancer. In some embodiments, the breast cancer is chemotherapy or radiotherapy resistant breast cancer, endocrine resistant breast cancer, trastuzumab resistant breast cancer, or breast cancer demonstrating primary or acquired resistance to CDK4/CDK6 inhibition. In some embodiments, the breast cancer is advanced or metastatic breast cancer. In some embodiments of each of the foregoing, the breast cancer is characterized by amplification and/or overexpression of CCNE1 and/or CCNE2.

In some embodiments, the cancer is FIR-positive breast cancer. In some embodiments, the breast cancer is ER-positive breast cancer. In some embodiments, the breast cancer is HR-positive, HER2-negative breast cancer. In some embodiments, the breast cancer is ER-positive, HER2-negative breast cancer. In some embodiments, the breast cancer is responsive to treatment with a CDK4/6 inhibitor. In some embodiments, the breast cancer is resistant to treatment with a CDK4/6 inhibitor. In some embodiments, the breast cancer has progressed despite treatment with a CDK4/6 inhibitor. In some embodiments, the CDK4/6 inhibitor is palbociclib. In some embodiments, the CDK4/6 inhibitor is ribociclib. In some embodiments, the breast cancer has progressed despite first treatment with palbociclib, ribociclib, and/or fulvestrant and second treatment with abemaciclib and/or fulvestrant. In some embodiments, the method further comprises administering an effective amount of a CDK4/6 inhibitor. In some embodiments, the CDK4/6 inhibitor is selected from palbociclib and ribociclib, or a combination thereof. In some embodiments, the CDK4/6 inhibitor is ribociclib. In some embodiments, the breast cancer has CCNE amplification and/or overexpression.

In some embodiments, the breast cancer is triple negative breast cancer.

In some embodiments, the cancer is ovarian cancer. In some such embodiments, the cancer is ovarian cancer characterized by amplification and/or overexpression of CCNE1 and/or CCNE2. In some such embodiments, the cancer is (a) ovarian cancer; (b) characterized by amplification and/or overexpression of cyclin E1 (CCNE1) or cyclin E2 (CCNE2); or (c) both (a) and (b). In some embodiments, the cancer is ovarian cancer.

In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein is administered as first line therapy. In other embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein is administered as second (or later) line therapy. In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein is administered as second (or later) line therapy following treatment with an endocrine therapeutic agent and/or a CDK4/CDK6 inhibitor In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein is administered as second (or later) line therapy following treatment with an endocrine therapeutic agent, e.g., an aromatase inhibitor, a SERM or a SERD. In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein is administered as second (or later) line therapy following treatment with a CDK4/CDK6 inhibitor. In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein is administered as second (or later) line therapy following treatment with one or more chemotherapy regimens, e.g., including taxanes or platinum agents. In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein is administered as second (or later) line therapy following treatment with HER2 targeted agents, e.g., trastuzumab.

In some embodiments, the disease or disorder associated with CDK2 is N-my c amplified neuroblastoma cells (see Molenaar, et al., Proc Natl Acad Sci USA 106(31): 12968-12973) K-Ras mutant lung cancers (see Hu, S., et al., Mol Cancer Ther, 2015, 14(11): 2576-85, and cancers with FBW7 mutation and CCNE1 overexpression (see Takada, et al., Cancer Res, 201777(18): 4881-4893).

In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein can be used to treat sickle cell disease and sickle cell anemia.

Examples of cancers that are treatable using a crystalline solid form of the free base of Compound (I) disclosed herein, the amorphous form of the free base of Compound (I) disclosed herein, or a crystalline solid form of a pharmaceutically acceptable salt of Compound (I) disclosed herein include, but are not limited to, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, endometrial cancer, carcinoma of the cervix, carcinoma of the vagina, carcinoma of the vulva. Hodgkin's Disease, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemias including acute myeloid leukemia, chronic myeloid leukemia, acute lymphoblastic leukemia, chronic lymphocytic leukemia, solid tumors of childhood, lymphocytic lymphoma, cancer of the bladder, cancer of the kidney or urethra, carcinoma of the renal pelvis, neoplasm of the central nervous system (CNS), primary CNS lymphoma, tumor angiogenesis, spinal axis tumor, brain stein glioma, pituitary adenoma, Kaposi's sarcoma, epidermoid cancer, squamous cell cancer, T-cell lymphoma, environmentally induced cancers including those induced by asbestos, and combinations of said cancers. The crystalline solid forms of the free base of Compound (I) disclosed herein, the amorphous form of the free base of Compound (I) disclosed herein, or the crystalline solid forms of a pharmaceutically acceptable salt of Compound (I) disclosed herein are also useful for the treatment of metastatic cancers.

In some embodiments, cancers treatable with a crystalline solid form of the free base of Compound (I) disclosed herein, the amorphous form of the free base of Compound (I) disclosed herein, or a crystalline solid form of a pharmaceutically acceptable salt of Compound (I) disclosed herein include, but are not limited to, melanoma (e.g., metastatic malignant melanoma, BRAF and HSP90 inhibition-resistant melanoma, skin cutaneous melanoma (SKCM), renal cancer (e.g., clear cell carcinoma), prostate cancer (e.g., hormone refractory prostate adenocarcinoma), breast cancer, colon cancer, lung cancer (e.g., non-small cell lung cancer and small cell lung cancer), squamous cell head and neck cancer (e.g., head and neck squamous cell carcinoma (NHSC), urothelial cancer (e.g., bladder) and cancers with high microsatellite instability (MSIhigh). Additionally, the disclosure includes refractory or recurrent malignancies whose growth may be inhibited using a crystalline solid form of the free base of Compound (I) disclosed herein, the amorphous form of the free base of Compound (I) disclosed herein, or a crystalline solid form of a pharmaceutically acceptable salt of Compound (I) disclosed herein.

In some embodiments, cancers that are treatable using a crystalline solid form of the free base of Compound (I) disclosed herein, the amorphous form of the free base of Compound (I) disclosed herein, or a crystalline solid form of a pharmaceutically acceptable salt of Compound (I) disclosed herein include, but are not limited to, solid tumors (e.g., prostate cancer, colon cancer, esophageal cancer, endometrial cancer, ovarian cancer, uterine cancer, renal cancer, hepatic cancer, pancreatic cancer, gastric cancer, breast cancer, lung cancer, cancers of the head and neck, thyroid cancer, glioblastoma, sarcoma, bladder cancer, etc.), hematological cancers (e.g., lymphoma, leukemia such as acute lymphoblastic leukemia (ALL), acute myelogenous leukemia (AML), chronic lymphocytic leukemia (CLL), chronic myelogenous leukemia (CML), DLBCL, mantle cell lymphoma, Non-Hodgkin lymphoma (including follicular lymphoma, including relapsed or refractory NI-L and recurrent follicular), Hodgkin lymphoma or multiple myeloma) and combinations of said cancers.

In some embodiments, cancers that are treatable using a crystalline solid form of the free base of Compound (I) disclosed herein, the amorphous form of the free base of Compound (I) disclosed herein, or a crystalline solid form of a pharmaceutically acceptable salt of Compound (I) disclosed herein include, but are not limited to, cholangiocarcinoma, bile duct cancer, triple negative breast cancer, rhabdomyosarcoma, small cell lung cancer, leiomyosarcoma, hepatocellular carcinoma (e.g., liver hepatocellular carcinoma (LIHC)), Ewing's sarcoma, brain cancer, brain tumor, astrocytoma, neuroblastoma, neurofibroma, basal cell carcinoma, chondrosarcoma, epithelioid sarcoma, eye cancer. Fallopian tube cancer, gastrointestinal cancer, gastrointestinal stromal tumors, hairy cell leukemia intestinal cancer, islet cell cancer, oral cancer, mouth cancer, throat cancer, laryngeal cancer, lip cancer, mesothelioma, neck cancer, nasal cavity cancer, ocular cancer, ocular melanoma, pelvic cancer, rectal cancer, renal cell carcinoma, salivary gland cancer, sinus cancer, spinal cancer, tongue cancer, tubular carcinoma, urethral cancer, and ureteral cancer.

In some embodiments, cancers treatable with a crystalline solid form of the free base of Compound (I) disclosed herein, the amorphous form of the free base of Compound (I) disclosed herein, or a crystalline solid form of a pharmaceutically acceptable salt of Compound (I) disclosed herein include Genomic Identification of Significant Targets in Cancer (GISTIC) and pheochromocytoma and paraganglioma (PCPG).

In some embodiments, cancers treatable with a crystalline solid form of the free base of Compound (I) disclosed herein, the amorphous form of the free base of Compound (I) disclosed herein, or a crystalline solid form of a pharmaceutically acceptable salt of Compound (I) disclosed herein include advanced/relapsed tumors; CCNE1 amplified platinum-resistant or platinum-refractory ovarian cancer; endometrial cancer (with prior platinum therapy) that has progressed following 2 or more lines of therapies; and gastric cancer (with prior platinum therapy) that has progressed following 2 or more lines of therapies; and ER+HER2− BC that has progressed despite CDK4/6i. In some embodiments, cancers treatable with a crystalline solid form of the free base of Compound (I) disclosed herein, the amorphous form of the free base of Compound (I) disclosed herein, or a crystalline solid form of a pharmaceutically acceptable salt of Compound (I) disclosed herein include Platinum-resistant or platinum-refractory CCNE1 amplified ovarian cancer; CCNE1 amplified endometrial cancer that has failed 2 or more lines of therapies; CCNE1 amplified advanced/relapsed tumors that do not belong to the other groups; ER+HER2− BC that has progressed despite CDK4/6i; platinum-resistant or platinum-refractory CCNE1 amplified ovarian cancer; and ER+HER2− BC that has progressed despite CDK4/6i.

In some embodiments, diseases and indications that are treatable using a crystalline solid form of the free base of Compound (I) disclosed herein, the amorphous form of the free base of Compound (I) disclosed herein, or a crystalline solid form of a pharmaceutically acceptable salt of Compound (I) disclosed herein include, but are not limited to hematological cancers, sarcomas, Jung cancers, gastrointestinal cancers, genitourinary tract cancers, liver cancers, bone cancers, nervous system cancers, gynecological cancers, and skin cancers.

Exemplary hematological cancers include lymphomas and leukemias such as acute lymphoblastic leukemia (ALL), acute myelogenous leukemia (AML), acute promyelocytic leukemia (APL), chronic lymphocytic leukemia (CLL), chronic myelogenous leukemia (CML), diffuse large B-cell lymphoma (DLBCL), mantle cell lymphoma. Non-Hodgkin lymphoma (including relapsed or refractory NHL and recurrent follicular), Hodgkin lymphoma, myeloproliferative diseases (e.g., primary myelofibrosis (PMF), polycythemia vera (PV), and essential thrombocytosis (ET)), myelodysplasia syndrome (MDS), T-cell acute lymphoblastic lymphoma (T-ALL) and multiple myeloma (MM).

Exemplary sarcomas include chondrosarcoma. Ewing's sarcoma, osteosarcoma, rhabdomyosarcoma, angiosarcoma, fibrosarcoma, liposarcoma, myxoma, rhabdomyoma, rhabdosarcoma, fibroma, lipoma, harmatoma, and teratoma.

Exemplary lung cancers include non-small cell lung cancer (NSCLC), small cell lung cancer (SCLC), bronchogenic carcinoma, squamous cell, undifferentiated small cell, undifferentiated large cell, adenocarcinoma, alveolar (bronchiolar) carcinoma, bronchial adenoma, chondromatous hamartoma, and mesothelioma. Exemplary gastrointestinal cancers include cancers of the esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma), and colorectal cancer.

Exemplary genitourinary tract cancers include cancers of the kidney (adenocarcinoma. Wilm's tumor [nephroblastoma]), bladder and urethra (squamous cell carcinoma, transitional cell carcinoma, adenocarcinoma), prostate (adenocarcinoma (PRAD), sarcoma), and testis (seminoma, teratoma, embryonal carcinoma, teratocarcinoma, choriocarcinoma, sarcoma, interstitial cell carcinoma, fibroma, fibroadenoma, adenomatoid tumors, lipoma).

Exemplary liver cancers include hepatoma (hepatocellular carcinoma), cholangiocarcinoma, hepatoblastoma, angiosarcoma, hepatocellular adenoma, and hemangioma.

Exemplary bone cancers include, for example, osteogenic sarcoma (osteosarcoma), fibrosarcoma, malignant fibrous histiocytoma, chondrosarcoma, Ewing s sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma, and giant cell tumors.

Exemplary nervous system cancers include cancers of the skull (osteoma, hemangioma, granuloma, xanthoma, osteitis deformans), meninges (meningioma, meningiosarcoma, gliomatosis), brain (astrocytoma, medulloblastoma, glioma, brain lower grade glioma (LGG), ependymoma, germinoma (pinealoma), glioblastoma, glioblastoma multiforme (GBM), oligodendroglioma, schwannoma, retinoblastoma, congenital tumors, and spinal cord (neurofibroma, meningioma, glioma, sarcoma), as well as neuroblastoma and Lhermitte-Duclos disease.

Exemplary gynecological cancers include cancers of the uterus (endometrial carcinoma), cervix (cervical carcinoma, cervical squamous cell carcinoma (CESC), pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), and fallopian tubes (carcinoma).

Exemplary skin cancers include melanoma, basal cell carcinoma, Merkel cell carcinoma, squamous cell carcinoma, Kaposi's sarcoma, moles dysplastic nevi, lipoma, angioma, dermatofibroma, and keloids. In some embodiments, diseases and indications that are treatable using a crystalline solid form of the free base of Compound (I) disclosed herein, the amorphous form of the free base of Compound (I) disclosed herein, or a crystalline solid form of a pharmaceutically acceptable salt of Compound (I) disclosed herein include, but are not limited to, sickle cell disease (e.g., sickle cell anemia), triple-negative breast cancer (TNBC), myelodysplastic syndromes, testicular cancer, bile duct cancer, esophageal cancer, and urothelial carcinoma.

### Combinations

Crystalline solid forms of the free base of Compound (I) disclosed herein, the amorphous form of the free base of Compound (I) disclosed herein, or crystalline solid forms of a pharmaceutically acceptable salt of Compound (I) disclosed herein may be administered as single agents or may be administered in combination with other anti-cancer therapeutic agents, in particular standard of care agents appropriate for the particular cancer.

The term “additional anticancer therapeutic agent” as used herein means any one or more therapeutic agent, other than a crystalline solid form of the free base of Compound (I) disclosed herein, the amorphous form of the free base of Compound (I) disclosed herein, or a crystalline solid form of a pharmaceutically acceptable salt of Compound (I) disclosed herein, that is or can be used in the treatment of cancer. In some embodiments, such additional anticancer therapeutic agents include compounds derived from the following classes: mitotic inhibitors, alkylating agents, antimetabolites, antitumor antibiotics, anti-angiogenesis agents, topoisomerase I and II inhibitors, plant alkaloids, hormonal agents and antagonists, growth factor inhibitors, radiation, signal transduction inhibitors, such as inhibitors of protein tyrosine kinases and/or serine/threonine kinases, cell cycle inhibitors, biological response modifiers, enzyme inhibitors, antisense oligonucleotides or oligonucleotide derivatives, cytotoxics, immuno-oncology agents, and the like.

In some embodiments, the additional anticancer agent is an endocrine agent, such as an aromatase inhibitor, a Selective Estrogen-Receptor Downregulator (SERD) or a Selective Estrogen Receptor Modulator (SERM).

In some embodiments, the additional anticancer agent is a PIK3CA inhibitor including, but not limited to, alpelisib (PIQRAY), BEBT-908, BPI-21668, buparlisib, inavolisib, TQB-3525, RLY-2608, miransertib, MEN-1611 LOXO-783, HS-10352, HH-CY133, gedatolisib, and fimepinostat.

In some embodiments, the additional anticancer agent is an antibody-drug conjugates including, but not limited to, Trastuzumab deruxtecan (Enhertu), Trastuzumab duocarmazine, Trastuzumab emtansine (Kadcyla), Upifitamab rilsodotin, mirvetuximab soravtansine, Tisotumab vedotin (Tivdak), Praluzatamab ravtansine, Sacituzumab govitecan or Sacituzumab Govitecan-hziy (Trodelvy), Datopotamab deruxtecan, Ladiratuzumab vedotin, Patritumab deruxtecan, STRO-002, MORab-202, DS-6000, Anetumab, avtansine, XMT-2056. Disitamab Vedotin (RC48-ADC, Aidexi).

In some embodiments, the additional anticancer agent is a PLK1 inhibitor including, but not limited to onvansertib, BI2536, BI6727, GSK461364A, TAK960, rigosertib.

In some embodiments, the additional anticancer agent is Estrogen PROTAC (ARV-471, H3B-5942).

In other embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein may be administered in combination with a standard of care agent. In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein may be administered in combination with endocrine therapy, e.g., agents such as letrozole, fulvestrant, tamoxifen, exemestane, or anastrozole. In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein may be administered in combination with a chemotherapeutic agent, e.g., docetaxel, paclitaxel, cisplatin, carboplatin capecitabine, gemcitabine, vinorelbine, or liposomal doxorubicin. In other embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein may be administered in combination with an anti-HER2 agent, e.g., trastuzumab or pertuzumab.

In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein may be administered in combination with an effective amount of carboplatin, ribociclib, fulvestrant, or a combination thereof.

In some embodiments, the additional anticancer agent is an anti-angiogenesis agent, including for example VEGF inhibitors, VEGFR inhibitors, TIE-2 inhibitors, PDGFR inhibitors, angiopoetin inhibitors. PKCb inhibitors. COX-2 (cyclooxygenase II) inhibitors, integrins (alpha-v/beta-3), MMP-2 (matrix-metalloproteinase 2) inhibitors, and MMP-9 (matrix-metalloproteinase 9) inhibitors. Preferred anti-angiogenesis agents include sunitinib (Sutent™) bevacizumab (Avastin™), axitinib (AG 13736), SU 14813 (Pfizer), and AG 13958 (Pfizer). Additional anti-angiogenesis agents include vatalanib (CGP 79787), Sorafenib (Nexavar™), pegaptanib octasodium (Macugen™), vandetanib (Zactima™), PF-0337210 (Pfizer), SU 14843 (Pfizer), AZD 2171 (AstraZeneca), ranibizumab (Lucentis™), Neovastat™ (AE 941), tetrathiomolybdata (Coprexa™). AMG 706 (Amgen), VEGF Trap (AVE 0005), CEP 7055 (Sanofi-Aventis), XL 880 (Exelixis), telatinib (BAY 57-9352), and CP-868,596 (Pfizer). Other anti-angiogenesis agents include enzastaurin (LY 317615), midostaurin (CGP 41251), perifosine (KRX 0401), teprenone (Selbex™) and UCN 01 (Kyowa Hakko). Other examples of anti-angiogenesis agents include celecoxib (Celebrex™), parecoxib (Dynastat™), deracoxib (SC 59046), lumiracoxib (Preige™), valdecoxib (Bextra™), rofecoxib (Vioxx™), iguratimod (Careram™). IP 751 (Invedus). SC-58125 (Pharmacia) and etoricoxib (Arcoxia™). Yet further anti-angiogenesis agents include exisulind (Aptosyn™), salsalate (Amigesic™), diflunisal (Dolobid™), ibuprofen (Motrin™), ketoprofen (Orudis™), nabumetone (Relafen™), piroxicam (Feldene™), naproxen (Aleve™, Naprosyn™), diclofenac (Voltaren™), indomethacin (Indocin™), sulindac (Clinoril™), tolmetin (Tolectin™), etodolac (Lodine™), ketorolac (Toradol™), and oxaprozin (Daypro™). Yet further anti-angiogenesis agents include ABT 510 (Abbott), apratastat (TMI 005), AZD 8955 (AstraZeneca), incyclinide (Metastat™), and PCK 3145 (Procyon).

Yet further anti-angiogenesis agents (including VEGFR/PDGFR inhibitors) include, but are not limited to, ponatinib (Iclusig), BT1718, anlotinib, lenvatinib (Lenvima), tivozanib (Fotivda), dovitinib, brolucizumab (Beovu), aflibercept (Eylea), and faricimab.

Yet further anti-angiogenesis agents include acitretin (Neotigason™), plitidepsin (Aplidine™), cilengtide (EMD 121974), combretastatin A4 (CA4P), fenretinide (4 HPR), halofuginone (Tempostatin™), Panzem™ (2-methoxyestradiol), PF-03446962 (Pfizer), rebimastat (BMS 275291), catumaxomab (Removab™), lenalidomide (Revlimid™), squalamine (EVIZON™), thalidomide (Thalomid™), Ukrain™ (NSC 631570), Vitaxin™ (MEDI 522), and zoledronic acid (Zometa™).

In other embodiments, the additional anti-cancer agent is a so-called signal transduction inhibitor (e.g., inhibiting how regulatory molecules that govern the fundamental processes of cell growth, differentiation, and survival communicated within the cell). Signal transduction inhibitors include small molecules, antibodies, and antisense molecules. Signal transduction inhibitors include for example kinase inhibitors (e.g., tyrosine kinase inhibitors or serine/threonine kinase inhibitors) and cell cycle inhibitors. More specifically signal transduction inhibitors include, for example, farnesyl protein transferase inhibitors, EGF inhibitor, ErbB-1 (EGFR), ErbB-2, pan erb, IGF1R inhibitors. MEK, c-Kit inhibitors, FLT-3 inhibitors, K-Ras inhibitors, PI3 kinase inhibitors, JAK inhibitors, STAT inhibitors, Raf kinase inhibitors. Akt inhibitors, mTOR inhibitor, P70S6 kinase inhibitors, inhibitors of the WNT pathway and so called multi-targeted kinase inhibitors. Additional examples of signal transduction inhibitors which may be used in conjunction with a compound of the invention and pharmaceutical compositions described herein include BMS 214662 (Bristol-Myers Squibb), lonafarnib (Sarasar™), pelitrexol (AG 2037), matuzumab (EMD 7200), nimotuzumab (TheraCIM h-R3™), panitumumab (Vectibix™), Vandetanib (Zactima™), pazopanib (SB 786034), ALT 110 (Alteris Therapeutics), BIBW 2992 (Boehringer Ingelheim), and Cervene™ (TP 38). Other examples of signal transduction inhibitors include gefitinib (Iressa™), cetuximab (Erbitux™), erlotinib (Tarceva™), trastuzumab (Herceptin™), sunitinib (Sutent™), imatinib (Gleevec™), crizotinib (Pfizer), lorlatinib (Pfizer), dacomitinib (Pfizer), bosutinib (Pfizer), gedatolisib (Pfizer), canertinib (CI 1033), pertuzumab (Omnitarg™), lapatinib (Tycerb™), pelitinib (EKB 569), miltefosine (Miltefosin™), BMS 599626 (Bristol-Myers Squibb), Lapuleucel-T (Neuvenge™), NeuVax™ (E75 cancer vaccine), Osidem™ (IDM 1), mubritinib (TAK-165), CP-724,714 (Pfizer), panitumumab (Vectibix™), ARRY 142886 (Array Biopharm), everolimus (Certican™), zotarolimus (Endeavor™), temsirolimus (Torisel™), AP 23573 (ARIAD), and VX 680 (Vertex), XL 647 (Exelixis), sorafenib (Nexavar™), LE-AON (Georgetown University), and GI-4000 (GlobeImmune). Other signal transduction inhibitors include ABT 751 (Abbott), alvocidib (flavopiridol). BMS 387032 (Bristol Myers). EM 1421 (Erimos), indisulam (E 7070), seliciclib (CYC 200). BIO 112 (Onc Bio), BMS 387032 (Bristol-Myers Squibb), palbociclib (Pfizer), and AG 024322 (Pfizer).

In other embodiments, the additional anti-cancer agent is a so called classical antineoplastic agent. Classical antineoplastic agents include but are not limited to hormonal modulators such as hormonal, anti-hormonal, androgen agonist, androgen antagonist and anti-estrogen therapeutic agents, histone deacetylase (HDAC) inhibitors. DNA methyltransferase inhibitors, silencing agents or gene activating agents, ribonucleases, proteosomics, Topoisomerase I inhibitors, Camptothecin derivatives, Topoisomerase II inhibitors, alkylating agents, antimetabolites, poly(ADP-ribose) polymerase-1 (PARP-1) inhibitor (such as, e.g., talazoparib, olapariv, rucaparib, niraparib, iniparib, veliparib), microtubulin inhibitors, antibiotics, plant derived spindle inhibitors, platinum-coordinated compounds, gene therapeutic agents, antisense oligonucleotides, vascular targeting agents (VTAs), and statins. Examples of classical antineoplastic agents used in combination therapy with a compound of the invention, optionally with one or more other agents include, but are not limited to, glucocorticoids, such as dexamethasone, prednisone, prednisolone, methylprednisolone, hydrocortisone, and progestins such as medroxyprogesterone, megestrol acetate (Megace), mifepristone (RU-486). Selective Estrogen Receptor Modulators (SERMs: such as tamoxifen, raloxifene, lasofoxifene, afimoxifene, arzoxifene, bazedoxifene, fispemifene, ormeloxifene, ospemifene, tesmilifene, toremifene, trilostane and CHF 4227 (Cheisi), Selective Estrogen-Receptor Downregulators (SERD's: such as fulvestrant, LSZ102, G1T48, RAD1901, elacestrant, GDC-9545, giredestrant, SAR439859, amcenestrant, AZD9833, camizestrant, LY3484356, Zn-c5, D-0502), exemestane (Aromasin), anastrozole (Arimidex), atamestane, fadrozole, letrozole (Femara), formestane; gonadotropin-releasing hormone (GnRH; also commonly referred to as luteinizing hormone-releasing hormone [LHRH]) agonists such as buserelin (Suprefact), goserelin (Zoladex), leuprorelin (Lupron), and triptorelin (Trelstar), abarelix (Plenaxis), cyproterone, flutamide (Eulexin), megestrol, nilutamide (Nilandron), and osaterone, dutasteride, epristeride, finasteride. Serenoa repens, PHL 00801, abarelix, goserelin, leuprorelin, triptorelin, bicalutamide; antiandrogen agents, such as enzalutamide, abiraterone acetate, bicalutamide (Casodex); and combinations thereof. Other examples of classical antineoplastic agents used in combination with a compound of the invention include but are not limited to suberolanilide hydroxamic acid (SAHA, Merck Inc./Aton Pharmaceuticals), depsipeptide (FR901228 or FK228), G2M-777, MS-275, pivaloyloxymethyl butyrate and PXD-101; Onconase (ranpimase), PS-341 (MLN-341), Velcade (bortezomib), 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, camptothecin, 10-hydroxycamptothecin, 9-aminocamptothecin, irinotecan, SN-38, edotecarin, topotecan, aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, Zinecard (dexrazoxane), nitrogen mustard N-oxide, cyclophosphamide. AMD-473, altretamine, AP-5280, apaziquone, brostallicin, bendamustine, busulfan, carboquone, carmustine, chlorambucil, dacarbazine, estramustine, fotemustine, glufosfamide, ifosfamide. KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, satrplatin, and combinations thereof.

In still other embodiments, the additional anti-cancer agent is a so called dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda™), cytosine arabinoside, Gemzar™ (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflomithine, hydroxyurea, leucovorin, nolatrexed (Thymitaq), triapine, trimetrexate, N-(5-[N-(3,4-dihydro-2-methyl-4-oxoquinazolin-6-ylmethyl)-N-methylamino]-2-thenoyl)-L-glutamic acid. AG-014699 (Pfizer Inc.). ABT-472 (Abbott Laboratories), INO-1001 (Inotek Pharmaceuticals), KU-0687 (KuDOS Pharmaceuticals) and GPI 18180 (Guilford Pharm Inc) and combinations thereof.

Other examples of classical antineoplastic cytotoxic agents include, but are not limited to, Abraxane (Abraxis BioScience, Inc.), Batabulin (Amgen), EPO 906 (Novartis), Vinflunine (Bristol-Myers Squibb Company), actinomycin D, bleomycin, mitomycin C, neocarzinostatin (Zinostatn), vinblastine, vincristine, vindesine, vinorelbine (Navelbine), docetaxel (Taxotere). Ortataxel, paclitaxel (including Taxoprexin a DHA/paciltaxel conjugate), cisplatin, carboplatin, Nedaplatin, oxaliplatin (Eloxatin), Satraplatin, Camptosar, capecitabine (Xeloda), oxaliplatin (Eloxatin), Taxotere alitretinoin, Canfosfamide (Telcyta™), DMXAA (Antisoma), ibandronic acid. L-asparaginase, pegaspargase (Oncaspar™), Efaproxiral (Efaproxyn™—radiation therapy), bexarotene (Targretin™), Tesmilifene (DPPE-enhances efficacy of cytotoxics), Theratope™ (Biomira), Tretinoin (Vesanoid™), tirapazamine (Trizaone™), motexafin gadolinium (Xcytrin™) Cotara™ (mAb), and NBI-3001 (Protox Therapeutics), polyglutamate-paclitaxel (Xyotax™) and combinations thereof. Further examples of classical antineoplastic agents include, but are not limited to, as Advexin (ING 201), TNFerade (GeneVec, a compound which express TNFalpha in response to radiotherapy). RB94 (Ba) for College of Medicine). Genasense (Oblimersen. Genta), Combretastatin A4P (CA4P), Oxi-4503, AVE-8062, ZD-6126, TZT-1027, Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin, Niacin (Advicor, Kos Pharmaceuticals), Caduet, Lipitor, torcetrapib, and combinations thereof.

In other embodiments, the additional anti-cancer agent is an epigenetic modulator, for example an inhibitor or EZH2, SMARCA4, PBRM1, ARID1A, ARID2, ARID1B, DNMT3A, TET2, MLL1/2/3, NSD1/2, SETD2, BRD4, DOT1L, HKMTsanti, PRMT1-9, LSD1, UTX, IDH1/2 or BCL6.

In further embodiments, the additional anti-cancer agent is an immunomodulatory agent, such as, but not limited to, an inhibitor of CTLA-4 (e.g., ipilimumab), PD-1 or PD-L1 (e.g., pembrolizumab, nivolumab, avelumab, atezolizumab, durvalumab, cemiplimab, or dosterlimab), LAG-3 (e.g., relatlimab, TIM-3, TIGIT, 4-1BB, OX40, GITR, CD40, or a CAR-T-cell therapy.

In some embodiments, the additional anticancer agent is an EGFR inhibitor such as afatinib, osimertinib, lapatinib, erlotinib, dacomitinib, poziotinib, neratinib or gefitinib or an EGFR antibody such as cetuximab, panitumumab, or necitumumab.

Alternatively a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein can be administered in combination with other anti-cancer agents that are not EGFR inhibitors e.g., in combination with MEK, including mutant MEK inhibitors (trametinib, cobimtetinib, binimetinib, selumetinib, refametinib); c-MET, including mutant c-Met inhibitors (savolitinib, cabozantinib, foretinib) and MET antibodies (emibetuzumab); mitotic kinase inhibitors (CDK4/6 inhibitors such as palbociclib, ribociclib, abemacicilb, lerociclib, trilaciclib, dalpiciclib, BPI-16350); anti-angiogenic agents e.g., bevacizumab, nintedanib; apoptosis inducers such as Bcl-2 inhibitors e.g., venetoclax, obatoclax, navitoclax and Mcl-1 inhibitors e.g., AZD-5991, AMG-176, S-64315; and mTOR inhibitors e.g., rapamycin, temsirolimus, everolimus, ridoforolimus.

In some embodiments, a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein can also be administered in combination with an effective amount of a second agent selected from the group consisting of palbociclib (e.g., ibrance®), ribociclib, abemaciclib, tamoxifen, letrozole, olaparib (e.g., Lynparza®), niraparib, carboplatin, cisplatin, paclitaxel, gemcitabine, megestrol acetate, medroxyprogesterone acetate, capecitabine (e.g., Xeloda®), regorafenib (e.g., Stivarga®), afatinib (e.g., Gilotrif®), osimertinib (e.g., Tagrisso®), gefitinib (e.g., Iressa®), erlotinib (e.g., Tarceva®), ramucirumab (e.g., Cyramza®), an EGFR inhibitor, pralsetinib, ABT-263 (navitoclax), MK-1775 (adavosertib), BAY-1895344, berzosertib, ceralasertib, SRA-737, LY2603618 (rabusertib), and trastuzumab (e.g., Herceptin®), or combinations thereof. The EGFR inhibitor may be selected from afatinib, osimertinib, lapatinib, erlotinib, dacomitinib, poziotinib, neratinib, gefitinib JBJ-04-125-02, alflutinib (AST 2818), aumolertinib (formerly almonertinib) (HS10296), BBT-176, BI-4020, BPI-361175, BPI-D0316, CH7233163, gilitertinib, icotinib. JND-3229, lazertinib, nazartinib (EGF 816), avitinib, PCC-0208027, rezivertinib (BPI-7711), TQB3804, zorifertinib (AZ-3759), or DZD9008: an EGFR antibody such as cetuximab, panitumumab, necitumumab, HLX07, JMT101; or a bispecific EGFR and MET antibody (e.g., amivantamab ((JNJ-61186372, JNJ-372)).

### Biomarkers and Pharmacodynamics Markers

The disclosure further provides predictive markers (e.g., biomarkers and pharmacodynamic markers, e.g., gene copy number, gene sequence, expression levels, or phosphorylation levels) to identify those human subjects having, suspected of having, or at risk of developing a disease or disorder associated with CDK2 for whom administering a CDK2 inhibitor (“a CDK2 inhibitor” as used herein refers to a crystalline solid form of the free base of Compound (I) disclosed herein, the amorphous form of the free base of Compound (I) disclosed herein, or a crystalline solid form of a pharmaceutically acceptable salt of Compound (I) disclosed herein) is likely to be effective.

### CCNE1

In one embodiment, the biomarker is CCNE1. In particular an amplification of the cyclin E1 (CCNE1) gene and/or an expression level of CCNE1 in a biological sample would indicate that the patient or subject could benefit from administration of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutically acceptable salt thereof.

CCNE1 is a cell cycle factor essential for the control of the cell cycle at the G1/S transition (Ohtsubo et al., 1995, Mol. Cell. Biol. 15:2612-2624). CCNE1 acts as a regulatory subunit of CDK2, interacting with CDK2 to form a serine/threonine kinase holoenzyme complex. The CCNE1 subunit of this holoenzyme complex provides the substrate specificity of the complex (Honda et al., 2005, EMBO 24:452-463). CCNE1 is encoded by the cyclin E1 (“CCNE1”) gene (GenBank Accession No. NM_001238). The amino acid sequence of human CCNE1 is found at GenBank Accession No. NP_001229/UniProtKB Accession No. P24864).

In one aspect, the present disclosure provides a method of treating a subject having, or at risk of developing, a disease or disorder associated with CDK2, comprising administering to the subject a therapeutically effective amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition disclosed herein, wherein the subject has an amplification of the CCNE1 gene and/or have an expression level of CCNE1 higher than a control expression level of CCNE1. In some embodiments, the disease or disorder associated with CDK2 is cancer.

Also provided herein is a method of treating a patient having an amplified expression level of CCNE1 and suffering from, or at risk of developing, a solid tumor cancer, comprising administering to the patient a therapeutically effective amount of a crystalline solid form of the free base of Compound (1) disclosed herein, the amorphous form of the free base of Compound (I) disclosed herein, or a crystalline solid form of a pharmaceutically acceptable salt of Compound (I) disclosed herein, or a pharmaceutical composition disclosed herein.

An amplification of the CCNE1 gene and/or an expression level of CCNE1 that is higher than a control expression level of CCNE1 is indicative/predictive that a human subject having or at risk of developing a disease or disorder associated with CDK2 will respond to a CDK2 inhibitor. In some embodiments, the expression level of CCNE1 may be the level of CCNE1 mRNA. In other embodiments, the expression level of CCNE1 may be the level of CCNE1 protein.

### Other Biomarkers

In some embodiments, the contemplated biomarker may be p16 (also known as cyclin-dependent kinase inhibitor 2A, cyclin-dependent kinase 4 inhibitor A, multiple tumor suppressor 1, and p16-INK4a), which acts as a negative regulator of the proliferation of normal cells b, interacting with CDK4 and CDK6. In other embodiments, the contemplated biomarker may be phosphorylation of Rb at the serine corresponding to amino acid position 780. Rb is a regulator of the cell cycle and acts as a tumor suppressor. Rb is activated upon phosphorylation by cyclin D-CDK4/6 at Ser780 and Ser795 and by cyclin E/CDK2 at Ser807 and Ser811.

The contemplated biomarker may also be selected from the group consisting of RB1, RBL1, RBL2, CDKN2A, CDKN1A, CDKN1B, FBXW7, CCNE1, CCNE2, CCNA1, CCNA2, CCND1, CCND2, CCND3, CDK2, CDK3, CDK4, CDK6, CDKN2A, CDNK1A, CDKN1B E2F1, E2F2, E2F3, MYC, MYCL, MYCN, EZH2, ER, HER2, HER3, HPV+, and EGFR.

### Biological Samples

Suitable biological samples for the methods described herein include any sample that contains blood or tumor cells obtained or derived from the human subject in need of treatment. For example, a biological sample can contain tumor cells from biopsy from a patient suffering from a solid tumor. A tumor biopsy can be obtained by a variety of means known in the art. Alternatively, a blood sample can be obtained from a patient suffering from a hematological cancer.

A biological sample can be obtained from a human subject having, suspected of having, or at risk of developing, a disease or disorder associated with CDK2. In some embodiments, the disease or disorder associated with CDK2 is a cancer (such as those described supra).

Methods for obtaining and/or storing samples that preserve the activity or integrity of molecules (e.g., nucleic acids or proteins) in the sample are well known to those skilled in the art. For example, a biological sample can be further contacted with one or more additional agents such as buffers and/or inhibitors, including one or more of nuclease, protease, and phosphatase inhibitors, which preserve or minimize changes in the molecules in the sample.

### Methods of Administration and Dosage Forms

The precise amount of compound administered to provide an “effective amount” to the subject will depend on the mode of administration, the type, and severity of the cancer, and on the characteristics of the subject, such as general health, age, sex, body weight, and tolerance to drugs. The skilled artisan will be able to determine appropriate dosages depending on these and other factors. When administered in combination with other therapeutic agents, e.g., when administered in combination with an anti-cancer agent, an “effective amount” of any additional therapeutic agent(s) will depend on the type of drug used. Suitable dosages are known for approved therapeutic agents and can be adjusted by the skilled artisan according to the condition of the subject, the type of condition(s) being treated and the amount of a crystalline solid form of the free base of Compound (I) disclosed herein, the amorphous form of the free base of Compound (I) disclosed herein, or a crystalline solid form of a pharmaceutically acceptable salt of Compound (I) disclosed herein being used by following, for example, dosages reported in the literature and recommended in the Physician's Desk Reference (57th Ed., 2003).

“Treating” or “treatment” refers to obtaining a desired pharmacological and/or physiological effect. The effect can be therapeutic, which includes achieving, partially or substantially, one or more of the following results: partially or substantially reducing the extent of the disease, condition or cancer, ameliorating or improving a clinical symptom or indicator associated with the disease, condition or cancer; delaying, inhibiting or decreasing the likelihood of the progression of the disease, condition or cancer; or decreasing the likelihood of recurrence of the disease, condition or cancer.

The term “effective amount” means an amount when administered to the subject which results in beneficial or desired results, including clinical results, e.g., inhibits, suppresses or reduces the symptoms of the condition being treated in the subject as compared to a control. For example, a therapeutically effective amount can be given in unit dosage form (e.g., 0.1 mg to about 50 g per day, alternatively from 1 mg to about 5 grams per day; and in another alternatively from 10 mg to 1 gram per day).

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intratumoral, intradermal, intraperitoneal, subcutaneous, orally, topically, intrathecally, inhalationally, transdermally, rectally, and the like. Administration techniques that can be employed with the agents and methods described herein are found in e.g., Goodman and Gilman. The Pharmacological Basis of Therapeutics, current ed.; Pergamon; and Remington's, Pharmaceutical Sciences (current edition), Mack Publishing Co., Easton. Pa.

In addition, a crystalline solid form of the free base of Compound (I) disclosed herein, the amorphous form of the free base of Compound (I) disclosed herein, or a crystalline solid form of a pharmaceutically acceptable salt of Compound (I) disclosed herein, or a pharmaceutical composition of the disclosure can be co-administered with other therapeutic agents. As used herein, the terms “co-administration”, “administered in combination with”, and their grammatical equivalents, are meant to encompass administration of two or more therapeutic agents to a single subject, and are intended to include treatment regimens in which the agents are administered by the same or different route of administration or at the same or different times. In some embodiments the one or more of the crystalline solid forms of the free base of Compound (I) disclosed herein, the amorphous form of the free base of Compound (I) disclosed herein, or the crystalline solid forms of a pharmaceutically acceptable salt of Compound (I) disclosed herein or a pharmaceutical composition of the disclosure will be co-administered with other agents. These terms encompass administration of two or more agents to the subject so that both agents and/or their metabolites are present in the subject at the same time. They include simultaneous administration in separate compositions, administration at different times in separate compositions, and/or administration in a composition in which both agents are present. Thus, in some embodiments, the crystalline solid forms of the free base of Compound (I) disclosed herein, the amorphous form of the free base of Compound (I) disclosed herein, or crystalline solid forms of a pharmaceutically acceptable salt of Compound (I) disclosed herein and the other agent(s) are administered in a single composition. In some embodiments, the crystalline solid forms of the free base of Compound (I) disclosed herein, the amorphous form of the free base of Compound (I) disclosed herein, or crystalline solid forms of a pharmaceutically acceptable salt of Compound (I) disclosed herein and the other agent(s) are admixed in the composition.

The particular mode of administration and the dosage regimen will be selected by the attending clinician, taking into account the particulars of the case (e.g. the subject, the disease, the disease state involved, the particular treatment). Treatment can involve daily or multi-daily or less than daily (such as weekly or monthly etc.) doses over a period of a few days to months, or even years. However, a person of ordinary skill in the art would immediately recognize appropriate and/or equivalent doses looking at dosages of approved compositions for treating a disease using the disclosed CDK2 inhibitors for guidance.

The crystalline solid forms of the free base of Compound (I) disclosed herein, the amorphous form of the free base of Compound (1) disclosed herein, or crystalline solid forms of a pharmaceutically acceptable salt of Compound (I) disclosed herein can be administered to a patient in a variety of forms depending on the selected route of administration, as will be understood by those skilled in the art. The solid forms of the present teachings may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time.

The pharmaceutical composition of the disclosure is formulated to be compatible with its intended route of administration. In an embodiment, the composition is formulated in accordance with routine procedures as a pharmaceutical composition adapted for intravenous, subcutaneous, intramuscular, oral, intranasal, or topical administration to human beings. In preferred embodiments, the pharmaceutical composition is formulated for intravenous administration.

Typically, for oral therapeutic administration, a crystalline solid form of the free base of Compound (I) disclosed herein, the amorphous form of the free base of Compound (I) disclosed herein, or a crystalline solid form of a pharmaceutically acceptable salt of Compound (I) disclosed herein may be incorporated with excipient and used in the form of ingestible tablets, buccal tablets, troches, capsules, elixirs, suspensions, syrups, wafers, and the like.

Typically for parenteral administration, solutions of a crystalline solid form of the free base of Compound (I) disclosed herein, the amorphous form of the free base of Compound (I) disclosed herein, or a crystalline solid form of a pharmaceutically acceptable salt of Compound (I) disclosed herein can generally be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms.

Typically, for injectable use, sterile aqueous solutions or dispersion of, and sterile powders of a crystalline solid form of the free base of Compound (I) disclosed herein, the amorphous form of the free base of Compound (I) disclosed herein, or a crystalline solid form of a pharmaceutically acceptable salt of Compound (I) disclosed herein for the extemporaneous preparation of sterile injectable solutions or dispersions are appropriate.

### Synthesis of Compound (I)

In another aspect, provided herein is a method of preparing Compound (I):

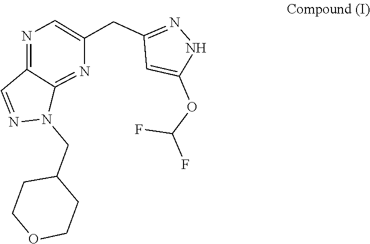 [SMILES: FC(F)Oc1cc(Cc2cnc3cnn(CC4CCOCC4)c3n2)n[nH]1]

comprising reacting a first compound represented by formula (1):

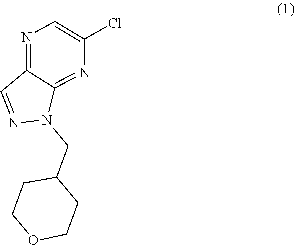 [SMILES: Clc1cnc2cnn(CC3CCOCC3)c2n1]

and a second compound represented by formula (2):

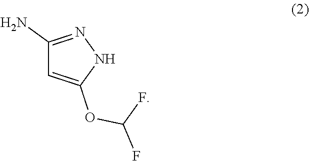

Compound (I) may be synthesized according to the following synthetic scheme A:

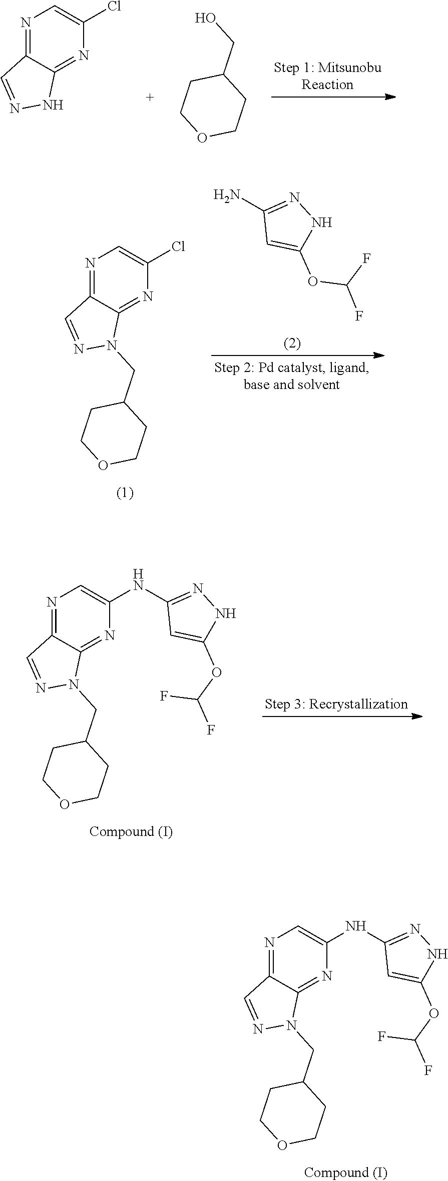 [SMILES: Clc1cnc2cn[nH]c2n1 ; OCC1CCOCC1 ; Clc1cnc2cnn(CC3CCOCC3)c2n1 ; Nc1cc(OC(F)F)[nH]n1 ; FC(F)Oc1cc(Nc2cnc3cnn(CC4CCOCC4)c3n2)n[nH]1 ; FC(F)Oc1cc(Nc2cnc3cnn(CC4CCOCC4)c3n2)n[nH]1]

Alternatively, Compound (I) may be synthesized according to the following synthetic scheme B:

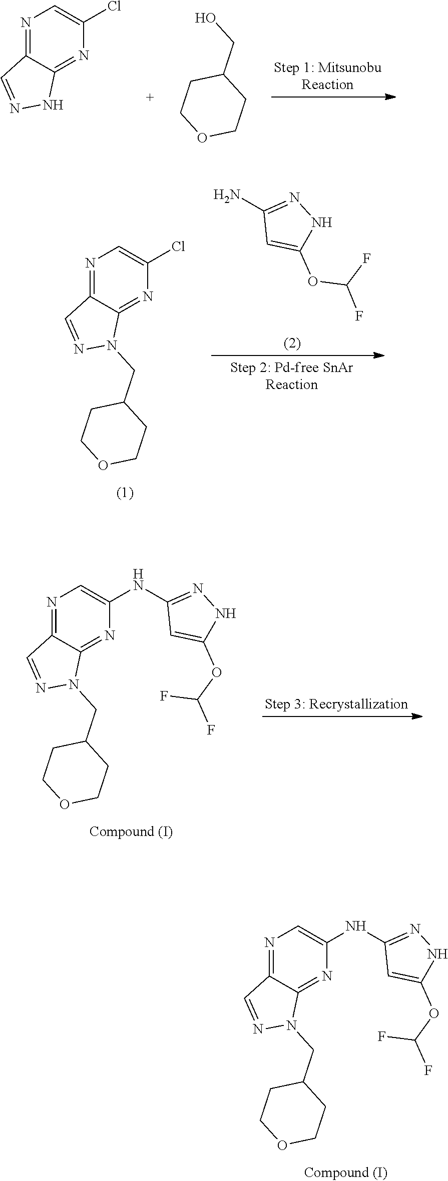 [SMILES: Clc1cnc2cn[nH]c2n1 ; OCC1CCOCC1 ; Clc1cnc2cnn(CC3CCOCC3)c2n1 ; Nc1cc(OC(F)F)[nH]n1 ; FC(F)Oc1cc(Nc2cnc3cnn(CC4CCOCC4)c3n2)n[nH]1 ; FC(F)Oc1cc(Nc2cnc3cnn(CC4CCOCC4)c3n2)n[nH]1]

In some embodiments of Step 1, the oxidizer of triphenylphosphine (TPP) is selected from the group consisting of diisopropyl azodicarboxylate (DIAD), di-(4-chlorobenzyl)azodicarboxylate (DCAD), di-tert-butyl azodicarboxylate (DBAD), and 4,4′-azopyridine. In some embodiments, the oxidizer of triphenylphosphine is DIAD.

In some embodiments of Step 1, the solvent is selected from the group consisting of ethyl acetate, isopropyl acetate, methyl tert-butyl ether, toluene, tetrahydrofuran, acetonitrile, 2-methyltetrahydrofuran, dichloromethane, and cyclohexane. In some embodiments, the solvent is ethyl acetate. In some embodiments, the solvent is toluene.

In some embodiments of Step 1, 6-chloro-1H-pyrazolo[3,4-b]pyrazine, tetrahydropyran-4-ylmethanol, and triphenylphosphine were combined in solvent followed by the addition of the oxidizer of triphenylphosphine (e.g., DIAD). In some embodiments of Step 1, 6-chloro-1H-pyrazolo[3,4-b]pyrazine, triphenylphosphine, and the oxidizer of triphenylphosphine were combined in solvent followed by the addition of tetrahydropyran-4-ylmethanol.

In some embodiments of Step 1, the reaction temperature was studied over the range of −40° C. to 25° C. In some embodiments, the reaction temperature was maintained at about −5 to about 5° C. until reaction completion. In some embodiments, the reaction temperature was maintained at about −10 to about 20° C. until reaction completion.

In some embodiments of Step 2, the reaction is a Buchwald-Hartwig coupling comprising a palladium catalyst, a ligand, a base, and a solvent. In some embodiments of Step 2, 5-(difluoromethoxy)-1H-pyrazol-3-amine (2) and solvent were combined followed by the addition of the palladium catalyst, ligand, and 6-chloro-1-((tetrahydro-2H-pyran-4-yl)methyl)-1H-pyrazolo[3,4-b]pyrazine (1). Then, a base was added to the mixture.

In some embodiments of Step 2, the palladium catalyst is selected from the group consisting of Pd(OAc)₂, [Pd(allyl)Cl]2, Pd(CH3CN)₂Cl₂, Pd₂(dba)₃, JackiePhos Pd G3, SL-J009-1 Pd G3, EtCPhos Pd G3, SPhos Pd G1, SPhos Pd G2, PEPPSI-iPr Pd, XPhos Pd G1, BrettPhos Pd G3, RuPhos Pd G2, KeYPhos Pd G2, CyDavePhos Pd G2, CyDavePhos Pd G3, and CyDavePhos Pd G4. In some embodiments, the palladium catalyst is selected from the group consisting of Pd(OAc)₂, [Pd(allyl)Cl]2, Pd(CH3CN)₂Cl₂, Pd₂(dba)₃. JackiePhos Pd G3, SL-J009-1 Pd G3, EtCPhos Pd G3, SPhos Pd G1, PEPPSI-iPr Pd, XPhos Pd G1, and BrettPhos Pd G3. In some embodiments, the palladium catalyst is [Pd(allyl)Cl]₂ or Pd₂(dba)₃. In some embodiments, the palladium catalyst is [Pd(allyl)Cl]₂. In some embodiments, the palladium catalyst is Pd₂(dba)₃.

In some embodiments of Step 2, the ligand is selected from the group consisting of XantPhos, BINAP-R, t-Bu-XPhos, SL-J009-1, DavePhos, XPhos, tBu-JohnPhos, BIPHEP, vBRIBP, RockPhos, tBu-BrettPhos, QPhos, SPhos, DPEPhos, CPhos, H8-BINAP-R, Tol-BINAP-R, DCyPF, DiPrPF, ChenPhos, SEGPHOS-R, Tedicyp, Quinap-R, KeYPhos, JoYPhos, TrYPhos, MOP-R, SL-J003-1, RuPhos, MePhos, AmPhos, Cy-vBRIDP, TrixiePhos, APhos, AlPhos, CX-PCy, CX-POMetBu, Cy-BippyPhos, MorDalPhos, Ad-BrettPhos, Cy-BrettPhos, Cy-DavePhos, JohnPhos, Cy-JohnPhos, vBRIDP, ChiraPhos-RR, ChenPhos-RSR, PCy3-HBF4, SL-A116-1, DM-SEGPHOS-R, DTBM-SEGPHOS-R, and xyl-BINAP-R. In some embodiments, the ligand is selected from the group consisting of XantPhos, BINAP-R, t-Bu-XPhos, SL-J009-1, DavePhos, XPhos, tBu-JohnPhos, BIPHEP, vBRIBP, RockPhos, tBu-BrettPhos, QPhos, SPhos, DPEPhos, CPhos, H8-BINAP-R, Tol-BINAP-R, DCyPF, DiPrPF, ChenPhos, SEGPHOS-R, Tedicyp, Quinap-R, KeYPhos, JoYPhos, TrYPhos, MOP-R, SL-J003-1, RuPhos, MePhos, AmPhos, Cy-vBRIDP, TrixiePhos, AlPhos, CX-PCy, and Cy-BippyPhos. In some embodiments, the ligand is selected from the group consisting of DavePhos, SPhos, CPhos, H8-BINAP-R, Tol-BINAP-R, RuPhos, MePhos, AmPhos, MorDalPhos, Cy-BrettPhos, Cy-DavePhos, and xyl-BINAP-R. In some embodiments, the ligand is selected from the group consisting of DavePhos, H8-BINAP-R, Tol-BINAP-R, and xyl-BINAP-R. In some embodiments, the ligand is DavePhos.

In some embodiments of Step 2 for a Buchwald-Hartwig coupling or for a palladium-free S_{N}Ar reaction, the base is selected from the group consisting of potassium tert-butoxide (KOtBu), sodium tert-butoxide (t-BuONa or NaOtBu), lithium tert-butoxide (LiOtBu), LiHMDS (lithium bis(trimethylsilyl)amide), NaHMDS (sodium bis(trimethylsilyl)amide), KHMDS (potassium bis(trimethylsilyl)amide), NaOAc (sodium acetate). KOAc (potassium acetate), K₃PO₄ (potassium phosphate), Na₂CO₃ (sodium carbonate), K₂CO₃ (potassium carbonate), Cs₂CO₃ (cesium carbonate), and lithium diisopropylamide (LDA). In some embodiments, the base is selected from the group consisting of KOtBu, NaOtBu, LiOtBu. LiHMDS, NaHMDS, KHMDS, KOAc, K₃PO₄, Cs₂CO₃, and LDA. In some embodiments for a Buchwald-Hartwig coupling or for a palladium-free S_{N}Ar reaction, the base is NaOtBu, LiOtBu, LiHMDS, NaHMDS, KHMDS, or LDA. In some embodiments, the base is NaOtBu. In some embodiments, the base is LiHMDS, NaHMDS, KHMDS, or LDA. In some embodiments, the base is LiHMDS.

In some embodiments of Step 2 for a Buchwald-Hartwig coupling or for a palladium-free S_{N}Ar reaction, the solvent is selected from the group consisting of tert-butanol (t-BuOH), tert-amyl alcohol, isopropanol, ethanol (EtOH), dioxane, dimethoxyethane (DME), toluene, N,N-dimethylformamide (DMF), tetrahydrofuran (THF), 2-methytetrahydrofuran (Me-THF), anisole, methyl tert-butyl ether (MTBE), N-methyl-2-pyrrolidone (NMP), 1,3-dioxolane, chlorobenzene, or a combination thereof. In some embodiments, the solvent is selected from the group consisting of t-BuOH, tert-amyl alcohol, isopropanol, EtOH, dioxane, DME, toluene, DMF, THF, Me-THF, anisole, MTBE, and NMP. In some embodiments, the solvent is selected from the group consisting of tert-butanol, tert-amyl alcohol, dioxane, DME, THF, and Me-THF. In some embodiments, the solvent is tert-butanol. In some embodiments, the solvent is THF.

In some embodiments of Step 2 when the reaction is a Buchwald-Hartwig coupling, one or more of the following impurities are formed:

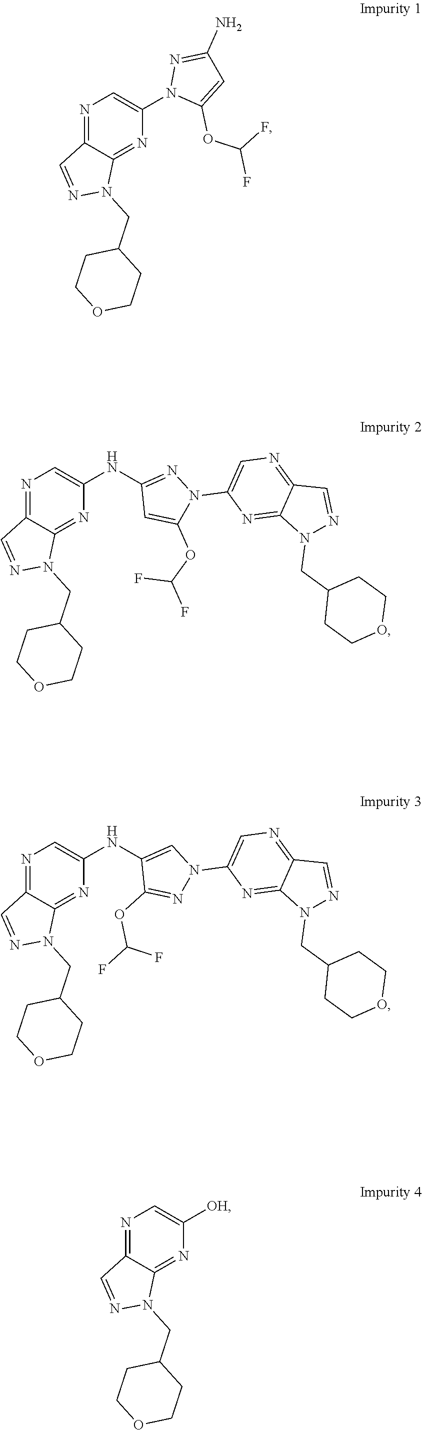 [SMILES: Nc1cc(OC(F)F)n(-c2cnc3cnn(CC4CCOCC4)c3n2)n1 ; FC(F)Oc1cc(Nc2cnc3cnn(CC4CCOCC4)c3n2)nn1-c1cnc2cnn(CC3CCOCC3)c2n1 ; FC(F)Oc1nn(-c2cnc3cnn(CC4CCOCC4)c3n2)cc1Nc1cnc2cnn(CC3CCOCC3)c2n1 ; Oc1cnc2cnn(CC3CCOCC3)c2n1]

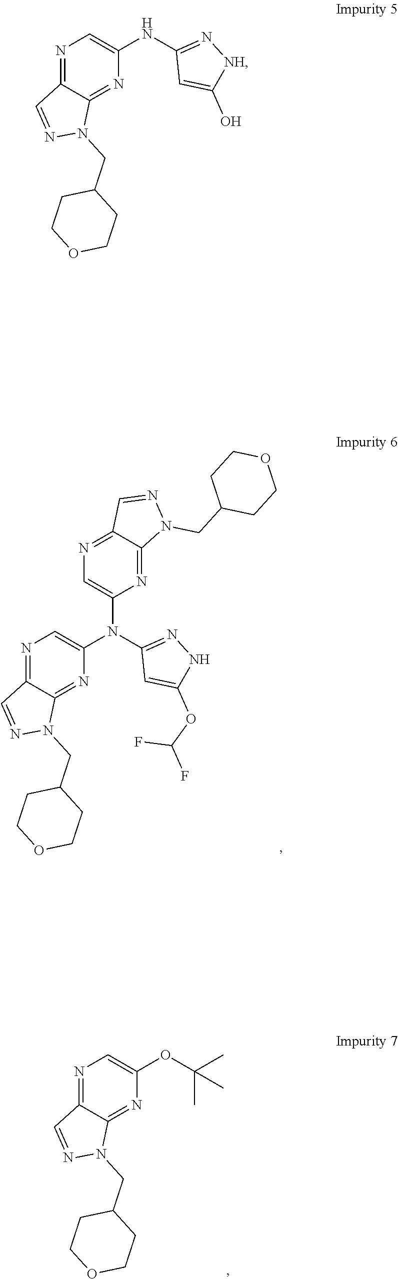 [SMILES: Oc1cc(Nc2cnc3cnn(CC4CCOCC4)c3n2)n[nH]1 ; FC(F)Oc1cc(N(c2cnc3cnn(CC4CCOCC4)c3n2)c2cnc3cnn(CC4CCOCC4)c3n2)n[nH]1 ; CC(C)(C)Oc1cnc2cnn(CC3CCOCC3)c2n1]

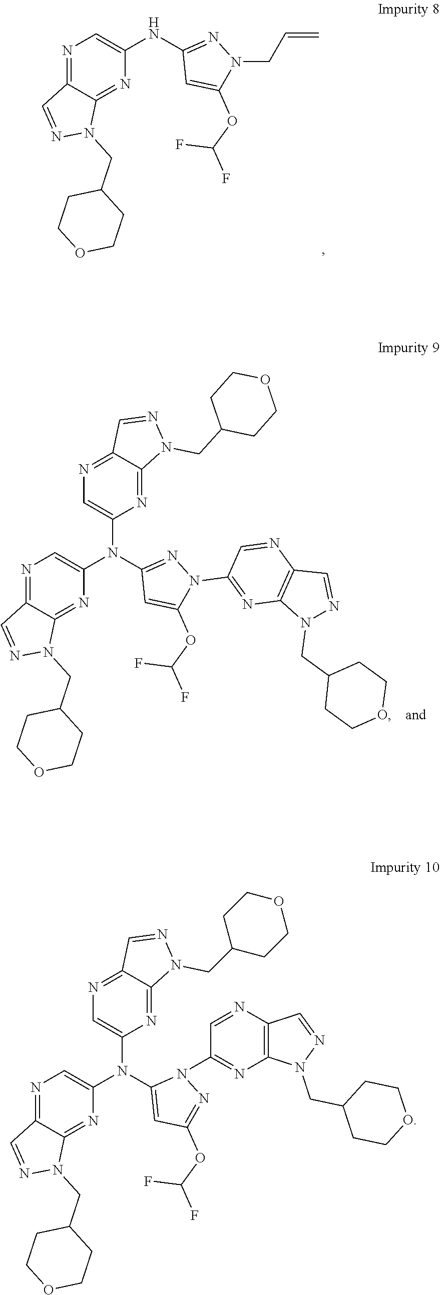

For example, the following impurities can be formed if certain processes are used

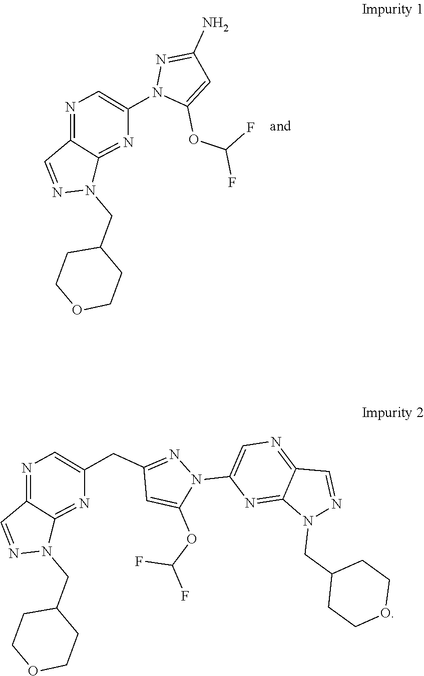 [SMILES: Nc1cc(OC(F)F)n(-c2cnc3cnn(CC4CCOCC4)c3n2)n1 ; FC(F)Oc1cc(Cc2cnc3cnn(CC4CCOCC4)c3n2)nn1-c1cnc2cnn(CC3CCOCC3)c2n1]

Some of the challenges with a Buchwald-Hartwig coupling with a palladium catalyst for use in the preparation of Compound (I) can include, but are not limited to, complex impurity generation, inefficient palladium removal via aqueous washes, product loss to aqueous waste, high cost of the palladium catalyst and/or ligand, high loading requirement of palladium catalyst and/or ligand, a hygroscopic base (e.g., sodium tert-butoxide), and/or a requirement of a palladium scavenger. In some embodiments, one or more of these challenges is eliminated with the removal of a palladium catalyst.

Provided herein, in certain embodiments, is a substantially pure compound (I). For example a composition comprising compound (I) is provided that may include less than 10%, less than 7%, less than 5%, or less than 3 wt %, based on the weight of the compound (or based on the weight of the composition), of one or more impurity compounds such as disclosed herein.

In another aspect, provided herein is a method of preparing Compound (I):

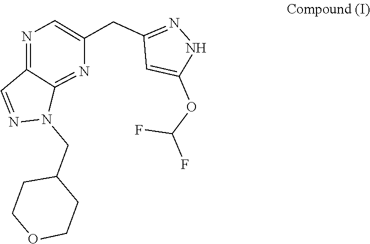 [SMILES: FC(F)Oc1cc(Cc2cnc3cnn(CC4CCOCC4)c3n2)n[nH]1]

comprising reacting a first compound represented by formula (1):

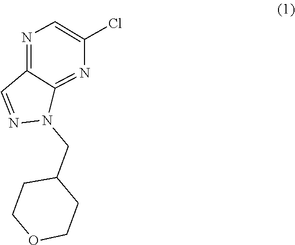 [SMILES: Clc1cnc2cnn(CC3CCOCC3)c2n1]

and a second compound represented by formula (2):

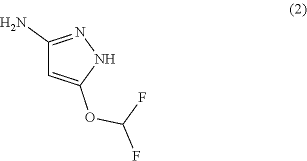

wherein reacting the first compound and the second compound further includes a base that activates the diamine compound (2) that then reacts with the heteroaryl chloride compound (1). Without being bound by any theory, the reaction is mediated by a base that may activate the diamine compound (2) to form a highly reactive dianion, which may react directly with the heteroaryl chloride compound (1). In some embodiments, the base is a strong non-nucleophilic base. In some embodiments, the base is a strong non-nucleophilic amide base. In some embodiments, the strong non-nucleophilic amide base is LiHMDS. NaHMDS, KHMDS, or LDA.

In some embodiments of Step 2, the reaction is a palladium-free S_{N}Ar reaction. In some embodiments of Step 2, compound (1) and compound (2) were combined in solvent followed by the addition of a strong non-nucleophilic base. In some embodiments of Step 2, the reaction is a palladium-free S_{N}Ar reaction. In some embodiments of Step 2, compound (1) and compound (2) were combined in solvent followed by the addition of a strong non-nucleophilic amide base (e.g., LiHMDS, NaHMDS, KHMDS, or LDA).

In some embodiments of Step 2 when the reaction is a palladium-free S_{N}Ar reaction, the reaction has improved selectivity, high conversion, and high overall purity. In some embodiments, the impurities formed with a Buchwald-Hartwig coupling are eliminated.

In some embodiments of Step 2 when the reaction is a palladium-free S_{N}Ar reaction, one or more of the following impurities are formed:

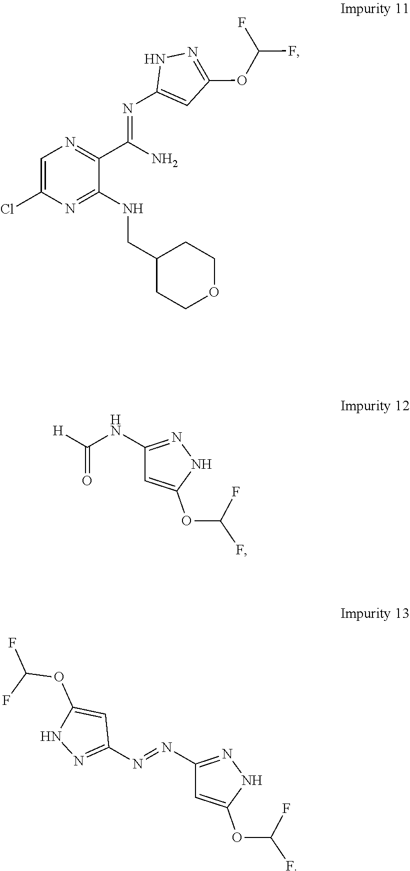 [SMILES: N/C(=N\c1cc(OC(F)F)n[nH]1)c1ncc(Cl)nc1NCC1CCOCC1 ; O=CNc1cc(OC(F)F)[nH]n1 ; FC(F)Oc1cc(/N=N/c2cc(OC(F)F)[nH]n2)n[nH]1]

The following examples are intended to be illustrative and are not intended to be limiting in any way to the scope of the disclosure.

### EXAMPLES

### Abbreviation

| | |
|---|---|
| Name | Abbreviation |

Solvents

| | |
|---|---|
| 2-Propanol | IPA |
| Acetonitrile | ACN |
| Dimethyl acetamide | DMAc |
| Dimethyl sulfoxide | DMSO |
| Ethanol | EtOH |
| Ethyl | EtOAc |
| acetate | |
| Isopropyl acetate | IPAc |
| Isopropyl alcohol | IPA |
| Methanol | MeOH |
| Methyl isobutyl ketone | MIBK |
| N,N-Dimethylacetamide | DMAc |
| tert-Butyl methyl ether | MtBE |
| Tetrahydrofuran | THF |
| 2-Methyltetrahydrofuran | 2-MeTHF |
| Trifluoroacetic acid | TFA |
| Trifluoroethanol | TFE |

Units

| | |
|---|---|
| Angstrom | Å or ang. |
| Celsius | C. |
| Degree | ° or deg. |
| Equivalent(s) | eq. |
| Gram | g |
| Hour | h |
| Hertz (s⁻¹) | Hz |
| Joule | J |
| Kelvin | K |
| Kilovolt | kV |
| Liter | L |
| Micrometer | μm |
| Micromole | μmol |
| Molar | M |
| Milliampere | mA |
| Milligram | mg |
| Milliliter | mL |
| Minute | min |
| Nanometer | nm |
| Second | s |
| Volume | vol. |
| Weight | wt |

Instruments

| | |
|---|---|
| Differential Scanning Calorimetry | DSC |
| Nuclear Magnetic Resonance | NMR |
| X-ray Powder Diffraction | XRPD |
| Thermogravimetric Analysis | TGA |

Others

| | |
|---|---|
| Amorphous | Am. |
| Broad peak | br. |
| Low crystalline | L.C. |
| Overnight | O/N |
| Relative humidity | RH |
| Room temperature (20-24° C.) | RT |

### Analysis Conditions:

¹H-Nuclear Magnetic Resonance Spectroscopy (¹H-NMR)

¹H NMR spectra were obtained with an Avance III 400 Bruker 400 MHz NMR (acquisition time=4.09 seconds with a 1 second delay; 16 scans) Unless otherwise indicated, all protons were reported in either DMSO-d6 or CDCl₃ NMR solvent as parts-per million (ppm) with respect to residual undeuterated NMR solvent signals (DMSO (2.50±0.02 ppm) and CDCl₃ (7.26±0.02 ppm)).

### Liquid Chromatography-Mass Spectrometry

The liquid chromatography-mass spectrometry (LC-MS) data (sample analyzed for purity and identity) were obtained with an Agilent product line 1290 Infinity II LC stack using an Agilent model G6135C LC/MSD XT spectrometer utilizing an AJS (ESI) ionization fitted with a Waters Atlantis T3 column (C18, 3.0 μm particle size, 4.6×150 mm dimensions) reverse-phase column at 22 degrees Celsius. The mobile phase consisted of a mixture of solvent 0.1% trifluoro acetic acid in water and acetonitrile. The respective acquisition method for each step was utilized.

### Differential Scanning Calorimetry (DSC) and Thermogravimetric Analysis (TGA)

DSC was performed using a Mettler Toledo DSC³⁺. The sample (3-5 mg) was weighed directly in a 40 μL hermetic aluminum pan with a pinhole. DSC was performed using a TA Discovery DSC. The sample (1-5 mg) was weighed directly in a 40 μL hermetic aluminum pan with a pinhole. TGA and DSC were performed on the same sample simultaneously using a Mettler Toledo TGA/DSC³⁺. Protective and purge gas was nitrogen at a flowrate of 20-30 mL/min and 50-100 mL/min respectively. The desired amount of sample (5-10 mg) was weighed directly in a hermetic aluminum pan with pinhole and analyzed according to the parameters below:

Parameters

| | Mettler Toledo | | TA Discovery | | Mettler Toledo | |
|---|---|---|---|---|---|---|
| | DSC³⁺ | | DSC | | TGA/DSC³⁺ | |
| Method | Ramp | | Ramp | | Ramp | |
| Sample size | 3-5 | mg | 1-5 | mg | 5-10 | mg |
| Heating rate | 10.0° | C./min | 10.0° | C./min | 10.0° | C./min |

| | | | |
|---|---|---|---|
| Temperature | 30 to 300° C. | 30 to 300° C. | 30 to 300° C. |
| range | | | |
| Method gas | N₂ at 60.00 | N₂ at 50.00 | |
| | mL/min | mL/min | |

### X-Ray Powder Diffraction (XRPD)

XRPD was performed using a Rigaku MiniFlex 600 in reflection mode (i.e. Bragg-Brentano geometry). Samples were prepared on Si zero-return wafers. The parameters for XRPD methods used are listed below:

| Parameter | Regular Scan |
|---|---|
| X-ray wavelength | Cu Kα1, 1.540598 Å, |
| X-ray tube setting | 40 kV, 15 mA |
| Slit condition | 1.25° div., Ni kβ filter, 0.3 mm rec. |
| Scan mode | Continuous |
| Scan range (°2θ) | 4-30 |
| Step size (°2θ) | 0.05 |
| Scan speed (°/min) | 0.23 |
| Spin | No |

XRPD of the free base and the salts disclosed herein was performed using a Bruker D8 Advance equipped with LYNXEYE detector in reflection mode (i.e. Bragg-Brentano geometry). Samples were prepared on Si zero-return wafers. The parameters for XRPD methods used are listed below:

| Parameter | Regular Scan | High Resolution Scan |
|---|---|---|
| X-ray wavelength | Cu Kα1, 1.540598 Å, | Cu Kα1, 1.540598 Å, |
| X-ray tube setting | 40 kV, 40 mA | 40 kV, 40 mA |
| Slit condition | 0.6 mm div. + 2.5° soller | 0.6 mm div. + 2.5° soller |
| Scan mode | Step | Step |
| Scan range (°2θ) | 4-30 | 4-40 |
| Step size (°2θ) | 0.03 | 0.02 |
| Dwell time (s/step) | 0.23 | 0.9 |
| Spin | Yes (0.5 Hz) | Yes (0.5 Hz) |

### Example 1 Preparation and Characterization of Selected Polymorph Forms of Free Base Compound (I)

### 1.1 Form A

### 1.1A: Synthesis of Compound (I) Based on Scheme A

### Step 1 Based on Scheme A:

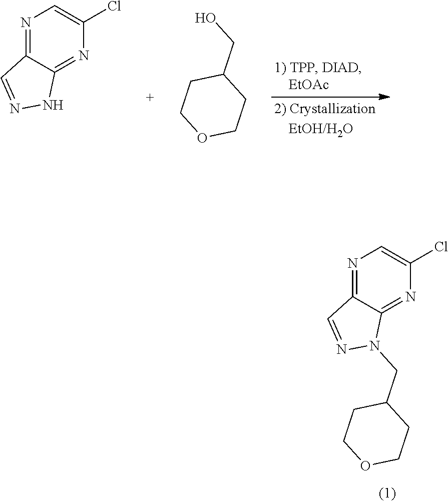 [SMILES: Clc1cnc2cn[nH]c2n1 ; OCC1CCOCC1 ; Clc1cnc2cnn(CC3CCOCC3)c2n1]

6-chloro-1H-pyrazolo[3,4-b]pyrazine (39.8 kg, 1.0 eq), tetrahydropyran-4-ylmethanol (35.9 kg, 1.2 eq), triphenylphosphine (TPP) (74.2 kg, 1.1 eq) and ethyl acetate (EtOAc) (260 L) were combined to form a suspension. The temperature of the suspension was adjusted to −5-3° C. Diisopropyl azodicarboxylate (DIAD) (62.4 kg, 1.2 eq) was added to the reaction mixture over 3.7 hours maintaining the temperature at −5-3° C. and the mixture stirred until reaction completion.

n-Heptane (520 L) was added at −5-10° C. and the resulting mixture kept at this temperature for 1.3 hour. The suspension was filtered, and the filter cake washed with a mixture of ethyl acetate (12 L) and n-heptane (110 L) at −5-10 C. The combined filtrate and wash were treated with magnesium chloride (9.8 kg, 0.4 eq) and the stirred mixture was heated to 45-55° C. for 1.7 hours then filtered. The cake was washed with mixture of ethyl acetate (4 L) and n-heptane (35 L). The combined filtrate and wash were treated with magnesium chloride (9.8 kg, 0.4 eq) at 45-55° C. for approximately 1 h. The slurry was filtered, and the cake washed with a mixture of ethyl acetate (4 L) and n-heptane (35 L). The combined filtrate and wash were concentrated at atmospheric pressure to a volume of 198 L. Ethanol (200 L) was added the mixture concentrated to 198 L. This process was repeated two more times and the final volume brought to 119 L. The mixture was cooled to 15-23° C. and the mixture seeded with the product (200 g, 0.5% w/w), cooled further to −7-3° C. and held for 13.5 h. The suspension was filtered, and the cake washed with 0-5° C. water (3×120 L). The material was dried at 50° C. under vacuum to give the desired compound (45.3 kg, 67% yield (assay corrected)) with 99.1% AUC purity.

¹H NMR: (400 MHz, CDCl₃) [ppm] δ8.51 (s, 1H), 8.25 (s, 1H), 4.35 (d, J=7.2 Hz, 2H), 3.96-3.92 (m, 2H), 3.37-3.30 (m, 2H), 2.34-2.24 (m, 1H), 1.47-1.44, (m, 4H).

LC-MS: m/z=253.1 [M+H]⁺

### Step 2 Based on Scheme A:

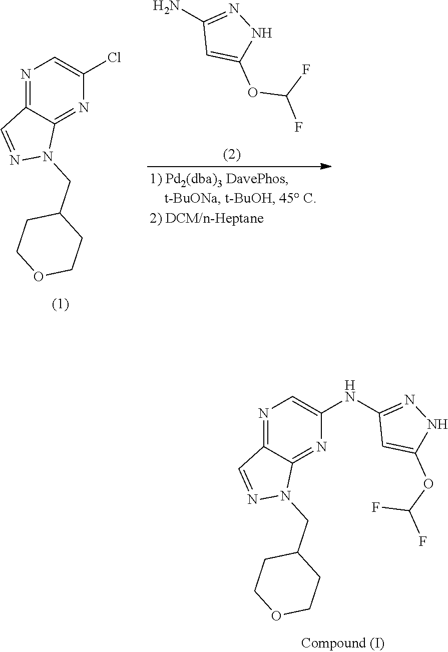 [SMILES: Clc1cnc2cnn(CC3CCOCC3)c2n1 ; Nc1cc(OC(F)F)[nH]n1 ; FC(F)Oc1cc(Nc2cnc3cnn(CC4CCOCC4)c3n2)n[nH]1]

5-(difluoromethoxy)-1H-pyrazol-3-amine (compound represented by formula (2)) (21.2 kg, 1.1 eq) and tert-butanol (t-BuOH) (123 kg) were combined in a vessel under a nitrogen atmosphere at 27-35° C. and the mixture sparged for 30 min. 6-chloro-1-((tetrahydro-2H-pyran-4-yl)methyl)-1H-pyrazolo[3,4-b]pyrazine (compound represented by formula (1)) (32.6 kg, 1.0 eq), tris(dibenzylideneacetone)dipalladium (0) (Pd₂(dba)₃) (4402 g, 0.04 eq), and 2-dicyclohexylphosphino-2′-(N,N-dimethylamino)biphenyl (DavePhos) (2448 g, 0.05 eq) were added to the mixture under nitrogen. Sodium tert-butoxide (t-BuONa) (3×10.0 kg, 2.1 eq) was added portion-wise keeping the temperature below 45° C. The mixture was heated to 45-55° C. until reaction completion. 2-Methytetrahydrofuran (157 L) was added, and the mixture concentrated under vacuum at <45° C. to 157 L. This sequence was repeated three more times to a final level of 157 L.

The mixture was diluted with 2-methyltetrahydrofuran (314 L) and the solution washed with a mixture of hydrochloric acid (36%, 9.1 kg, 0.66 eq) in aqueous sodium chloride (7.9 kg NaCl in 157 L water) at 30-37° C. The organic layer was separated and washed in three portions with a solution of sodium bicarbonate (40.4 kg) in water (471 L) and the aqueous layers discarded. The organic layer was concentrated under vacuum at <45° C. to a minimum stirring level, diluted with dichloromethane (157 L) and the mixture concentrated to a minimum stirring level. This sequence was repeated twice more. The mixture was cooled to <30° C. and diluted with dichloromethane (35 L) to reach a level of 157 L.

The resulting mixture was heated to 40-45° C., diluted with n-heptane (55 L), held for 30 minutes then cooled to 10-25° C. over 10 hours. The slurry was further cooled to 5-10° C., held for 3 hours and the solids collected by filtration. The solids were washed with a mixture of dichloromethane (30 L) and n-heptane (30 L) at 5-10° C. and dried under vacuum at 50° C. to give the desired compound (35.6 kg, 67% yield (assay corrected)) with 92.0% AUC purity.

¹H NMR: (400 MHz, DMSO-d6) [ppm] δ 12.2 (s, 1H), 10.8 (s, 1H), 8.20 (s, 1H), 8.15 (s, 1H), 7.3 (t, J=73.6 Hz, 1H), 6.0 (s, 1H), 4.4 (m, 2H), 3.8 (m, 2H), 3.2 (m, 2H), 2.2 (m, 1H), 1.4-1.3 (m, 4H).

LC-MS: m/z=366.2 [M+H]⁺

### Step 3 Based on Scheme A:

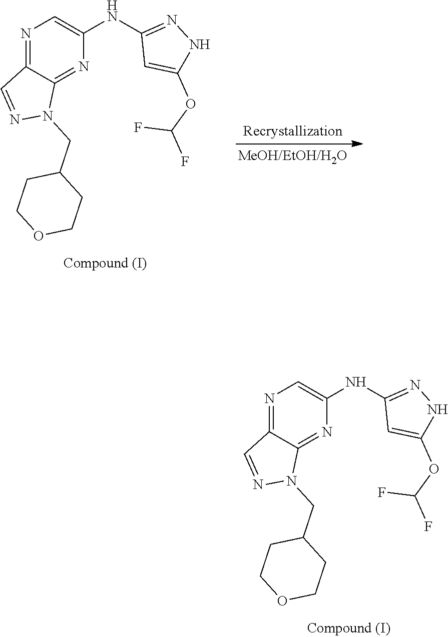 [SMILES: FC(F)Oc1cc(Nc2cnc3cnn(CC4CCOCC4)c3n2)n[nH]1 ; FC(F)Oc1cc(Nc2cnc3cnn(CC4CCOCC4)c3n2)n[nH]1]

The crude material (35.4 kg, 1 eq) was combined with methanol (520 L) and heated to 50-55° C. until complete dissolution. The solution was cooled to 40-50° C. and treated with SiliaMetS Thiol (34.8 kg) for 2 hours. The suspension was filtered through a pad of Celite, and the cake washed with methanol (70 L) and combined with the filtrate. This sequence was repeated twice more, and the resulting solution was passed through a 0.2-micron filter. The solution was concentrated under vacuum at <45° C. to a level of 284 L. The mixture was cooled to 17-35° C. and was combined with ethanol (32 L) and water (70 L). The mixture was heated to 60-70° C. until complete dissolution was observed. Water (35 L) was added to the solution at 60-70° C. The solution was cooled to 32.5-37.5° C. over 1 hour, seeded with the product (0.18 kg, 0.5% w/w) and held for 2 hours. The suspension was cooled to 15-25° C. over 2 hours and held for 12 hours. The suspension was cooled to 7-12° C. over 50 minutes and water (35 L) was added over 10 minutes. The mixture was held at 7-12° C. for 3 hours, and the solids collected by filtration. The solids were washed with a mixture of water (43 L) and methanol (28 L) at 7-12° C. The material was dried under vacuum at 50° C. to give the desired compound (26.5 kg, 72% yield (assay corrected)) with 99.1% AUC purity.

¹H NMR: (400 MHz, DMSO-d6) [ppm] δ 12.2 (s, 1H), 10.8 (s, 1H), 8.20 (s, 1H), 8.14 (s, 1H), 7.3 (t, J=73.6 Hz, 1H), 6.0 (s, 1H), 4.4 (m, 2H), 3.8 (m, 2H), 3.2 (m, 2H), 2.2 (m, 1H), 1.4-1.3 (m, 4H).

LC-MS: m/z=366.2 [M+H]⁺

### 1.1B: Process Optimization of Reaction Conditions for the Preparation of Compound (I)

Several different catalysts, ligands, bases, solvents, and reaction temperatures were investigated to further optimize the reaction conditions for the preparation of Compound (I). The results of conditions that were studied for the coupling of (1) and (2) (Scheme A, Step 2 (Table 17) and Scheme B, Step 2 (Table 18)) are described below.

The catalyst was formed in the reaction vial by combining allylpalladium(II) chloride dimer ([Pd(allyl)Cl]₂) (10 μmol, 0.025 eq) and ligands bis-phosphine (20 μmol, 0.05 eq) and mono-phosphine (40 μmol, 0.1 eq) in solvent for 30 minutes before removing solvent under high vacuum. (1) (400 μmol, 1.0 eq) and (2) (400 μmol, 1 eq) were added as a solution in tetrahydrofuran to the reaction vial before removing solvent under high vacuum. Sodium tert-butoxide (t-BuONa) (1200 μmol, 3 eq) was added as a solution in tetrahydrofuran to the reaction vial before removing solvent under high vacuum. Solvent (1 mL) was added, and the mixture was held at 45° C. for 18 hours with agitation. The screening results identified reaction conditions of a palladium catalyst using a DavePhos ligand with sodium tert-butoxide (NaOtBu) as a base in tert-butanol as a solvent, which match the reagents used in Example 1.1A, Step 2. Further screening based on these reaction conditions identified BINAP ligands using NaOtBu as the base and ether solvents.

TABLE 17
Summary of certain impurities based on certain
reaction conditions using Scheme A, Step 2.

| Ligand | Solvent | Compound (I) | Impurity 2 | Impurity 5 |
|---|---|---|---|---|
| H8-BINAP-R | Dioxane | 79.2% | 1.5% | 5.0% |
| H8-BINAP-R | THF | 72.4% | 2.4% | 7.1% |
| H8-BINAP-R | DME | 72.7% | 5.0% | 4.7% |
| tol-BINAP-R | Dioxane | 76.6% | 1.6% | 5.0% |
| tol-BINAP-R | THF | 73.2% | 2.8% | 6.4% |
| tol-BINAP-R | DME | 71.3% | 5.3% | 4.3% |
| xyl-BINAP-R | Dioxane | 76.7% | 1.9% | 5.3% |
| xyl-BINAP-R | Me—THF | 73.2% | 2.3% | 6.1% |
| H8-BINAP-R | Me—THF | 76.1% | 2.4% | 6.3% |
| tol-BINAP-R | Me—THF | 72.4% | 2.6% | 6.1% |

Further screening of alternate bases lead to the discovery of an S_{N}Ar reaction. For the Step 2 coupling reaction, it was discovered herein that certain strong non-nucleophilic bases (e.g., strong non-nucleophilic amide bases, including but not limited to, LiHMDS, NaHMDS, KHMDS, or LDA) allowed for a coupling reaction without a palladium catalyst or an associated ligand (Scheme B). (1) (100 mg, 1.0 eq) and (2) (59 mg, 1.0 eq) were combined in solvent (1.5 mL). A base was added while the reaction was maintained at certain target temperatures and monitored by HPLC. Only certain strong non-nucleophilic amide bases provided rapid, selective, and high conversion with high overall purity to afford the desired Compound (I).

TABLE 18
Summary of results based on certain reaction conditions using Scheme B, Step 2.

| Base/amount | Solvent | Temp. | | Reaction Time | Results |
|---|---|---|---|---|---|
| LiHMDS (3.3 eq) | THF | 0° | C. | 1 h | 100% conversion within 1 h |
| DIPEA (2.0 eq) | dioxane | 100° | C. | 48 h | No conversion to Compound (I) |
| NaH (2.0 eq) | DMF | 0° | C. | | No conversion to Compound (I); |
| | | | | | conversion to Impurities 1 and 2 |
| K₂CO₃ (2.0 eq) | t-amyl | 105° | C. | 16 h | No conversion to Compound (I); |

| | | | | |
|---|---|---|---|---|
| | alcohol | | | high conversion to Impurity 1 |
| K₂CO₃ (2.0 eq) | ACN | reflux | 48 h | No conversion to Compound (I); |
| | | | | high conversion to Impurity 1 |

### 1.1C: Synthesis of Compound (I) Based on Scheme B

### Step 1 Based on Scheme B:

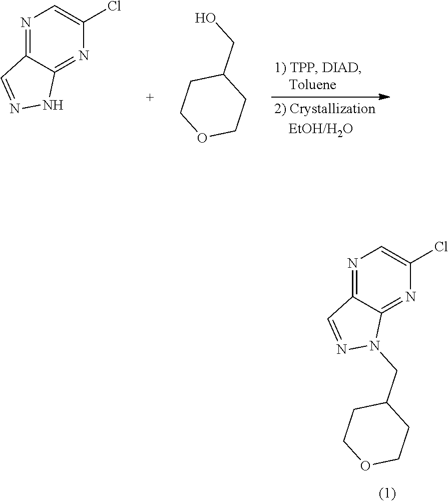 [SMILES: Clc1cnc2cn[nH]c2n1 ; OCC1CCOCC1 ; Clc1cnc2cnn(CC3CCOCC3)c2n1]

6-chloro-1H-pyrazolo[3,4-b]pyrazine (50.0 kg, 1.0 eq), tetrahydropyran-4-ylmethanol (45.1 kg, 1.2 eq), triphenylphosphine (TPP) (93.4 kg, 1.1 eq) and toluene (479 L) were combined to form a suspension. The temperature of the suspension was adjusted to −10-0° C. Diisopropyl azodicarboxylate (DIAD) (78.5 kg, 1.2 eq) was added to the reaction mixture over 10 hours while maintaining the temperature at −10-0° C. The mixture was stirred at −10-−5° C. for an additional 3 hours, and it was then adjusted to 15-25° C. and stirred for 6 hours until reaction completion.

The reaction mixture was distilled down to a total volume of ~300 L at ≤60° C. and then adjusted to 35-45° C. n-Heptane (520 L) was added at 35-45° C. and the resulting mixture was kept at this temperature for 5.5 hours. The suspension was filtered, and the filter cake was washed with a mixture of toluene (29 L) and n-heptane (117 L) at 35-45° C. The combined filtrate and wash were adjusted to 50-60° C. and then treated with magnesium chloride (30.5 kg, 1.0 eq). The resulting mixture was stirred at 50-60° C. for 7.5 h and then filtered. The cake was washed with a mixture of toluene (40 L) and n-heptane (161 L) at 50-60° C. The combined filtrate and wash were distilled down to a volume of ~250 L at ≤60° C. Ethanol (EtOH) (247 L) was then added, and the solvent swap cycle was repeated 2 additional times to a final target volume of ~219 L. Ethanol (51 L) was added to the resulting mixture and the temperature was adjusted to 40-50° C. Water (103 L) was added while maintaining temperature, and the resulting solution was slowly cooled to 10-20° C. over 2 hours. The cooled solution was seeded with the product (250 g, 0.5% w/w), cooled further down to −10-−5° C. and held for 5.5 hours. Water (150 L) was added at −10-−5° C. and the suspension was stirred for 2 hours at temperature. The suspension was filtered, and the cake was washed with cold (−10-−5° C.) 1:4 ethanol:water (2×150 L). The material was dried at 35-45° C. under vacuum for 3 hours to give the desired compound (61.9 kg, 76% yield) with 99.8% AUC purity.

¹H NMR: (400 MHz, CDCl₃) [ppm] δ8.51 (s, 1H), 8.25 (s, 1H), 4.35 (d, J=7.2 Hz, 2H), 3.96-3.92 (m, 2H), 3.37-3.30 (m, 2H), 2.34-2.24 (m, 1H), 1.47-1.44, (m, 4H).

LC-MS: m/z=253.1 [M+H]⁺

### Step 2 Based on Scheme B:

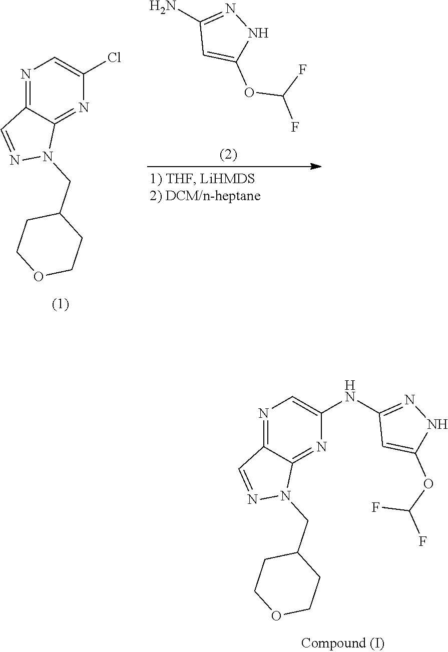 [SMILES: Clc1cnc2cnn(CC3CCOCC3)c2n1 ; Nc1cc(OC(F)F)[nH]n1 ; FC(F)Oc1cc(Nc2cnc3cnn(CC4CCOCC4)c3n2)n[nH]1]

6-chloro-1-((tetrahydro-2H-pyran-4-yl)methyl)-1H-pyrazolo[3,4-b]pyrazine (1) (100 mg, 1.0 eq), 5-(difluoromethoxy)-1H-pyrazol-3-amine (2) (59 mg, 1.0 eq) and anhydrous THF (1.5 mL) were combined to form a solution. At ambient temperature, lithium hexamethyldisilazane (LiHMDS) as a 1 M solution in THF (1.19 mL, 3.0 eq) was added to the reaction in one aliquot. The temperature was adjusted to 50° C., and the reaction mixture was stirred at 50° C. for 4 h where the reaction showed very clean and high conversion with a crude product with 87.1% AUC purity.

Alternatively, (1) (61.6 kg, 1.0 eq (2) (40.0 kg, 1.1 eq) and anhydrous tetrahydrofuran (THF) (367 L) were combined to form a solution. The temperature was adjusted to 0-5° C. While maintaining temperature, LiHMDS as a 24% solution in THF (597 Kg, 3.3 eq) was added over a period of 7 hours. The reaction mixture was stirred at −5 to 5° C. for 2 hours.

Upon reaction completion, the mixture was adjusted to −10-10° C. While maintaining temperature, 3 M hydrochloric acid (518 L) was added over a period of 9 hours. The temperature was adjusted to 15-25° C. and the aqueous layer was discarded. While maintaining temperature, 5% aqueous sodium bicarbonate (308 Kg) was added; the biphasic mixture was stirred for one hour and the aqueous layer was discarded. While maintaining temperature, 5% aqueous sodium bicarbonate (308 Kg) was added once more; the biphasic mixture was stirred for one hour and the aqueous layer was discarded. The resulting mixture was distilled down to a total volume of −308 L at <66° C. Dichloromethane (DCM) (185 L) was then added, and the resulting mixture was distilled down to a total volume of ~308 L at <66° C. The solvent swap cycle was repeated 8 additional times. The reaction mixture was then distilled down to a total of volume of ~246 L and the temperature was adjusted to 25-35° C. Dichloromethane (60 L) was added, and the temperature was adjusted to 35-45° C. While maintaining temperature, n-heptane (108 L) was added. The reaction mixture was slowly cooled down to 10-25° C. over a period of 10.5 hours, and then slowly cooled down to 5-10° C. over a period of 2.5 hours. The resulting suspension was stirred at temperature for 3.5 hours. The suspension was filtered, and the cake was washed with cold (5-10° C.) 1:1 dichloromethane: n-heptane (2×124 L). The material was dried at 45-55° C. under vacuum for 6 hours. The dry cake was slurried on the filter with water (308 L) at 35-45° C. After deliquoring, the wet cake was washed with n-heptane (2×126 L) at 15-30° C. The material was dried at 45-55° C. under vacuum for 12.5 hours to give the desired compound (78.4 kg, 88% yield) with 99.8% AUC purity.

### Step 3 Based on Scheme B:

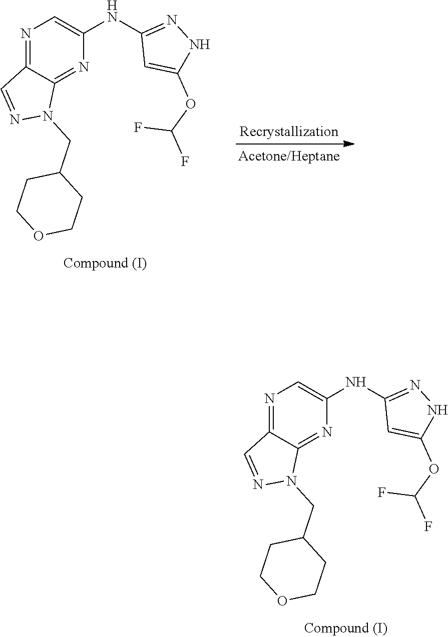 [SMILES: FC(F)Oc1cc(Nc2cnc3cnn(CC4CCOCC4)c3n2)n[nH]1 ; FC(F)Oc1cc(Nc2cnc3cnn(CC4CCOCC4)c3n2)n[nH]1]

The crude material (77.9 kg, 1.0 eq) was combined with acetone (1,172 L) and heated to 45-55° C. until complete dissolution. The resulting solution was polish-filtered and transferred to another reactor. The filtration assembly was rinsed with warm (45-55° C.) acetone (2×160 L), and the rinse was combined with the product filtrate. The resulting solution was distilled down to a total volume of ~818 L at <75° C. and then adjusted to reflux conditions at 55-60° C. for one hour. The reaction mixture was then cooled to 45-55° C. and seeded with the product (390 g, 0.5% w/w). The mixture was held at temperature for 2 hours and it was distilled down to a total volume of ~467 L at <75° C. The mixture was then held at 45-55° C. for 4 hours prior to addition of n-heptane (240 L) at temperature. The resulting slurry was then held at 45-55° C. for an additional 2 hours. n-Heptane (706 L) was added while maintaining temperature. The mixture was then held at 45-55° C. for 2 hours and slowly cooled down to 15-25° C. over a period of 4 hours. The mixture was slowly cooled down to 0-10° C. over a period of 1.5 hours and held at temperature for 6 hours. The resulting suspension was filtered, and the cake was washed with cold (0-10° C.) 1:2 acetone:n-heptane (2×152 L). The material was dried at 45-55° C. under vacuum for 10 hours to give the desired compound (65.1 kg, 84% yield) with 99.9% AUC purity.

TABLE 19
Comparison of Compound (I) prepared using Scheme A (Buchwald-
Hartwig coupling) vs. Scheme B (palladium-free S_{N}Ar reaction)

| | Scheme A | Scheme B |
|---|---|---|
| Step 1 Purity (HPLC AUC) | 99.1% | 99.8% |
| Step 1 Yield | 67% | 76% |
| Step 2 Purity (HPLC AUC) | 92.0% | 99.8% |
| Step 2 Yield | 67% | 88% |
| Purity (HPLC AUC) after recrystallization | 99.1% | 99.9% |
| Step 3 Yield after recrystallization | 72% | 84% |

Alternatively, The crude material (5.07 g, 1 eq) was combined with 12 vol acetone (60.8 mL) and heated to 50° C. until complete dissolution. The solution was heated to 75° C. and concentrated to level of ~6 vol (30.4 mL). The mixture was cooled to 50° C., and 3 vol n-heptane (16.8 mL) was added over 1.5 h (2 vol/h). The mixture was held for 2 h, then another 8.9 vol n-heptane (45.1 mL) was added over 4.5 h (2 vol/h), and held again for 2 h. The solution was cooled to 5° C. over 4.5 hours and held for 5 hours. The solids collected by filtration and were washed with a mixture of acetone:n-heptane (35:65 vol %) at 5° C. The material was dried under vacuum at 50° C. to give the desired compound (4.31 kg, 85.1% yield (w/w)) with 99.3% AUC purity.

The XRPD shows that the product obtained from Step 3 is in crystalline Form A (FIG. 1). The DSC has endotherms with onset peaks at about 191.7±2° C. and about 197.9±2° C. and an exotherm between them with an onset at about 194.9±2° C. (FIG. 2).

Alternatively, 7 vol. of MeOH:water (8:2 vol.) was added to Compound (I) free base, and the mixture was stirred and heated to about 65° C. until dissolution (about 10 minutes). The temperature of the solution was reduced to about 60° C., and water (1 vol.) was added. The solution was seeded with 1 wt. % of crystalline Form A and was stirred for about 1 h at about 60° C. Water (11 vol.) was added at a rate of 2 vol./h for a final composition of MeOH:water (3:7 vol.). The slurry was cooled from about 60° C. to about 10° C. over 5 hours at a rate of 10° C./h, and the resulting slurry was stirred O/N at 10° C. The slurry was filtered and was washed with 3 vol. of cold MeOH:water (3:7 vol.). The wet cake material was dried under vacuum (−29 inHg) overnight at 50° C.

Alternatively, about 25-30 mg of Compound (I) free base was dissolved in solvent at 50° C. until dissolution.

For slow cooling experiments the solutions (i.e., in 30 volumes of MeOH, in 49 volumes of EtOAc, in 18 volumes of acetone, or 13 volumes of IPA:water (9:1 volumes)) were cooled to 5° C. at 5° C. per hour with mixing over 9 h. This was accomplished by reducing the cooling manifold temperature by 0.083° C. per minute. Experiments were held at 5° C. until solids were observed, which were then recovered for XRPD analysis to yield crystalline Form A.

For fast cooling, the solutions (i.e., in 30 volumes of MeOH, in 49 volumes of EtOAc, in 18 volumes of acetone, or 9 volumes of acetone:water (7:3 volumes)) were transferred to an ice-water bath near 0° C. without mixing. After 10 min in the ice-water bath, mixing was resumed. If precipitation was observed, slurries were filtered immediately. If solids did not precipitate from solution at 0° C. for 2 h, the solutions were further cooled to −20° C. by placing in a freezer without mixing to yield crystalline Form A.

Alternatively, about 25 mg of Compound (I) free base was dissolved in 0.5 mL of THF, then 1.0 mL of MtBE was added over 60 min with mixing to yield crystalline Form A.

Alternatively, 101.2 mg of Compound (I) free base was suspended in 1 mL of acetone:water (1:1 vol.) to form a slurry that was stirred overnight at RT. The slurry was filtered, and solids were dried under vacuum at 50° C. for 3 h to yield pure crystalline Form A.

Alternatively, about 25-30 mg of Compound (I) free base was suspended in acetone:water (7:3 vol.) to form a slurry that was stirred overnight at 50° C. The slurry was filtered and optionally dried to yield pure crystalline Form A.

### 1.2 Form B

402.5 mg of Compound (I) free base was dissolved in 10 mL of acetone, and a stir bar was added. The slurry was heated on a hot plate at 50° C. until dissolved, and the solution was syringe filtered through a 0.45 m filter into a tared 20 mL vial. The solution was stirred while a light stream of nitrogen gas was blown into the vial. After 5 min, there were some solids visible in the vial, so the mixture was seeded with a spatula tip of Form B. After 10 min, the solvent had evaporated, and the vial was dried under vacuum (−29 inHg) overnight at 50° C.

Alternately, 400.7 mg of Compound (I) free base was sonicated in 10 mL of acetone (25 vol) to form a pale-yellow solution. The solution was stirred at RT while a stream of nitrogen gas was blown into the vial. Within 15 min, the solvent had evaporated. The vial was dried under vacuum (−29 inHg) at 50° C. for 2.5 h.

The XRPD analysis confirmed that the product obtained is crystalline Form B (FIG. 3). The DSC has a broad endotherm with an onset peak at about 198.7±2° C. (FIG. 4).

### 1.3 Form C

409.1 mg of Compound (I) free base was stirred in 20 mL of ACN:water (1:1 vol.) at 50° C. for 5 min until a thin, hazy slurry was formed. This mixture was split evenly into two new 20 mL vials, and 2 mL of ACN:water (1:1 vol.) was added. The mixtures dissolved completely and were syringe filtered with 0.45 m filters into new 20 mL vials. These filtered solutions were frozen in liquid nitrogen for 5 min and lyophilized overnight. XRPD analysis of the solids generated by lyophilization yielded crystalline Form C.

The XRPD analysis confirmed that the product obtained is crystalline Form C (FIG. 5). The DSC has three broad endotherms with onset peaks at about 30.6±2° C., about 170.5±2° C. and about 198.4±2° C. and two broad exotherms with onset peaks at about 176.8±2° C. and 253.7±2° C. (FIG. 6).

### 1.4 Form D

After 11 vol. of water was added to Compound (I) free base, some particles were observed. The final composition of IPA:water (3:7 vol) was reached for Compound (I) after 13 vol. was added, but only a very thin, hazy slurry was observed. This thin slurry was stirred at 50° C. for 45 min, then cooled to RT by reducing the temperature by 2.5° C. every 15 min (over 2 h). A flowable slurry was observed when the temperature reached 46° C.

The XRPD analysis confirmed that the product obtained is crystalline Form D (FIG. 7). The DSC has two broad endotherms with onset peaks at about 90.6±2° C. and about 200.1±2° C. (FIG. 8).

### 1.5 Form E

Crystalline Form F was dried at RT under active vacuum for up to 28 h to produce crystalline Form E. The XRPD analysis confirmed that the product obtained is crystalline Form E (FIG. 9). The DSC has four broad endotherms with onset peaks at about 89.9±2° C., about 100.1±2° C., about 190.9±2° C., and about 199.9±2° C. and a broad exotherm with an onset peak at about 242.0±2° C. (FIG. 10).

### 1.6 Form F

Crude material of Compound (I) free base was dissolved in MeOH:water (9:1 vol.), and water (5 to 10 vol. to reach 50 vol. % to 70 vol. % water in the system) was added at 35° C. or 50° C. at a rate of 1 vol./h to 3 vol./h and then cooled to 10° C. The obtained wet cake solid was crystalline Form F. The XRPD analysis confirmed that the product obtained is crystalline Form F (FIG. 11).

Alternatively, crude material of Compound (I) free base was dissolved in MeOH:EtOH:water (6.87 vol., 0.9 vol, and 2.0 vol., respectively). Water (1 vol.) was added at 65° C. followed by adding more water (1 vol.) at 35° C. over 30 min., and then the solvent mixture was cooled to 10° C. The obtained wet cake solid was crystalline Form F.

### 1.7 Amorphous Form

About 100 mg of Compound (I) free base in each of four vials were placed on a hot plate, and the temperature was increased to 220° C. The temperature was held at 220° C. for 5 min, then the vials were immediately placed in a container of liquid nitrogen and held for 5 min. The vials were then allowed to come to RT on the bench for over 10 min. Each vial was sampled for XRPD, which was indicative of amorphous solids for each sample.

### Example 2 Preparation and Characterization of Selected Polymorph Forms of Compound (I) Salts

### 2.1 Toluene-4-Sulfonic Acid Salt or Tosylate Salt Form 6-B

762.7 mg of Compound (I) free base was dissolved in 25 mL of TFE for a concentration of 30.51 mg/mL. 25 mg of Compound (I) free base in 819.5 μL of TFE was added to 203.0 mg of toluene-4-sulfonic acid in 10 mL of EtOH. The mixture was allowed to stir at 40° C. uncapped overnight to evaporate the solvent. The following day, the temperature was increased to 45° C., and a stream of nitrogen gas was used to evaporate the remaining solvent. The vials were then dried under vacuum (−29 inHg) at 50° C. overnight to dry the solids. EtOH was added (0.5 mL) to the dry solids, and the mixture was stirred at 45° C. for 2 h followed by stirring for at least 2 h at RT. The slurry was filtered and plated for XRPD analysis. The plate with the wet material was dried under vacuum (−29 inHg) at 50° C. for 2 h and the dry solids were analyzed by XRPD.

The XRPD analysis confirmed that the product obtained is crystalline toluene-4-sulfonic acid salt Form 6-B of Compound (I) (FIG. 13). The DSC has five broad endotherms with onset peaks at about 30.8±2° C., about 102.5±2° C. about 106.3±2° C., about 157.3±2° C., and about 185.8±2° C. and two exotherms with onset peaks at about 173.1±2° C. and about 177.6±2° C. (FIG. 14).

### 2.2 Toluene-4-Sulfonic Acid Salt or Tosylate Salt Form 6-C

25 mg of Compound (I) free base in 819.5 μL of TFE was added to 203.0 mg of toluene-4-sulfonic acid in 10 mL of EtOH. The mixture was allowed to stir at 40° C. uncapped overnight to evaporate the solvent. The following day, the temperature was increased to 45° C., and a stream of nitrogen gas was used to evaporate the remaining solvent. The vial was then dried under vacuum (−29 inHg) at 50° C. overnight to dry the solids. IPA:water (9:1 vol.) was added (0.5 mL) to the dry solids, and the mixture was stirred at 45° C. for 2 h followed by stirring for at least 2 h at RT. The slurry was filtered and plated for XRPD analysis. The plate with the wet materials was dried under vacuum (−29 inHg) at 50° C. for 2 h and the dry solids were analyzed by XRPD.

The XRPD analysis confirmed that the product obtained is crystalline toluene-4-sulfonic acid salt Form 6-C of Compound (I) (FIG. 15). The DSC has a broad endotherm with an onset peak at about 165.5±2° C. (FIG. 16).

### 2.3 Toluene-4-Sulfonic Acid Salt or Tosylate Salt Form 6-D

233.9 mg (1.1 eq.) of toluene-4-sulfonic acid was dissolved in 6 mL (15 vol.) of EtOH:MtBE (1:1 vol). The resulting solution was added to 400.6 mg of Compound (I) free base as a solid via pipet, and the resulting mixture was stirred at RT with an additional 2 mL (5 vol) of EtOH:MtBE (1:1 vol). After 2 h, the slurry yielded crystalline toluene-4-sulfonic acid salt Form 6-D. The slurry could be stirred for 2-3 days, and no changes to the XRPD were observed. The slurry was filtered and washed twice with 2 vol of EtOH:MtBE (1:1 vol). The solids were transferred to a tared vial and weighed, then the wet cake was dried at 50° C. under vacuum (−29 inHg) overnight. The following day, the dry solids were weighed with a yield of 495.1 mg (84 mol %).

The XRPD analysis confirmed that the product obtained is crystalline toluene-4-sulfonic acid salt Form 6-D of Compound (I) (FIG. 17). The DSC has two broad endotherms with onset peaks at about 172.3±2° C. and about 195.3±2° C. and a broad exotherm with an onset peak at about 177.9±2° C. (FIG. 18).

### 2.4 Ethanesulfonic Acid Salt or Ethanesulfonate Salt Form 7-A

100.25 μL (1.1 eq.) of ethanesulfonic acid was dissolved in 6 mL (15 vol.) of EtOH. The resulting solution was added to 400.3 mg of Compound (I) free base as a solid via pipet, and the resulting mixture was stirred at RT. After 2 h, the slurry yielded free base crystalline Form B. The slurry was seeded with a spatula tip of crystalline ethanesulfonic acid salt Form 7-A. The slurry was stirred over the weekend. The slurry was filtered and washed twice with 2 vol of EtOH. The solids were transferred to a tared vial and weighed, then the wet cake was dried at 50° C. under vacuum (−29 inHg) overnight. The following day, the dry solids were weighed with a yield of 478.9 mg (92 mol %).

The XRPD analysis confirmed that the product obtained is crystalline ethanesulfonic acid salt Form 7-A of Compound (I) (FIG. 19). The DSC has two broad endotherms with onset peaks at about 163.9±2° C. and about 183.8±2° C. and a broad exotherm with an onset peak at about 177.9±2° C. (FIG. 20).

### 2.5 Naphthalene-2-Sulfonic Acid Salt or Naphthalene-2-Sulfonate Salt Form 8-A

256.6 mg (1.1 eq.) of naphthalene-2-sulfonic acid was dissolved in 6 mL (15 vol.) of EtOAc. The resulting solution was added to 400.0 mg of Compound (I) free base as a solid via pipet, and the resulting mixture was stirred at RT with an additional 2 mL (5 vol) of EtOAc. After 2 h, the slurry yielded crystalline naphthalene-2-sufonic acid salt Form 8-A. The slurry could be stirred for 2-3 days, and no changes to the XRPD were observed. The slurry was filtered and washed twice with 2 vol of EtOAc. The solids were transferred to a tared vial and weighed, then the wet cake was dried at 50° C. under vacuum (−29 inHg) overnight. The following day, the dry solids were weighed with a yield of 519.3 mg (83 mol %).

The XRPD analysis confirmed that the product obtained is crystalline naphthalene-2-sulfonic acid salt Form 8-A of Compound (I) (FIG. 21). The DSC has a broad endotherm with an onset peak at about 199.9±2° C. (FIG. 22).

### 2.6 Hydrochloric Acid Salt or Hydrochloride Salt Form 1-B

About 25 mg of Compound (I) free base was dissolved in 819.5 μL of TFE. A volume equal to 1.1 eq. of HCl in 313.3 μL was added. EtOAc was added, and the mixture was allowed to stir at 40° C. uncapped overnight to evaporate the solvent. The following day, the temperature was increased to 45° C. and a stream of nitrogen gas was used to evaporate the remaining solvent from each vial. The vials were then dried under vacuum (−29 inHg) at 50° C. for at least 3 h.

The XRPD analysis confirmed that the product obtained is crystalline hydrochloride salt Form 1-B of Compound (I) (FIG. 23). The DSC has two broad endotherms with onset peaks at about 199.7±2° C. and about 204.2±2° C. (FIG. 24). Alternatively, the solid recovered ethanol-2M HCl in DEE solvent-antisolvent addition was generated by a slow crystallization. Initial antisolvent addition caused no crystallization. After about 1 hour, a bright orange precipitate formed slowly.

1. A crystalline solid form of the free base of Compound (I), wherein Compound (I) is represented by the following structural formula: 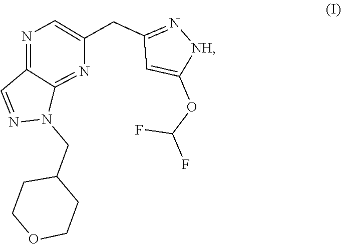 [SMILES: FC(F)Oc1cc(Cc2cnc3cnn(CC4CCOCC4)c3n2)n[nH]1]
wherein the crystalline solid form is referred to as crystalline solid Form A and is characterized by an X-ray powder diffraction (XRPD) pattern comprising three, four, or five peaks at the diffraction angles (2θ) selected from the group consisting of 5.9°±0.2, 17.6°±0.2, 18.1°±0.2, 23.1°±0.2, and 25.10°±0.2.

2. A crystalline solid form of the free base of Compound (1), wherein the crystalline solid form is referred to as crystalline solid Form A and is characterized by an XRPD pattern comprising three, four, or five peaks at the diffraction angles (2θ) selected from the group consisting of 5.9°±0.2, 17.6°±0.2, 21.4°±0.2, 25.1°±0.2, and 27.3°±0.2.

3. The crystalline solid form of claim 1 or 2, wherein the crystalline solid form is characterized by an XRPD pattern comprising three, four, five, six, seven, eight, nine, or ten peaks at the diffraction angles (2θ) selected from the group consisting of 5.9°±0.2, 17.6°±0.2, 18.1°±0.2, 19.1°±0.2, 21.4°±0.2, 23.1°±0.2, 25.1°±0.2, 26.6°±0.2, 27.3°±0.2, and 29.4°±0.2.

4. The crystalline solid form of any one of claims 1-3, wherein the crystalline solid form is characterized by an XRPD pattern comprising three, four, five, six, seven, eight, nine, ten, eleven, twelve, thirteen, fourteen, or fifteen peaks at the diffraction angles (2θ) selected from the group consisting of 5.9°±0.2, 17.3°±0.2, 17.6°±0.2, 18.1°±0.2, 18.8°±0.2, 19.0°±0.2, 19.1°±0.2, 21.0°±0.2, 21.4°±0.2, 23.1°±0.2, 25.1°±0.2, 25.5°±0.2, 26.6°±0.2, 27.3°±0.2, and 29.4°±0.2.

5. The crystalline solid form of any one of claims 1-4, wherein the crystalline solid form is characterized by an XRPD pattern having characteristic peaks at the diffraction angles (2θ) at 5.9°±0.2, 17.6°±0.2, and 25.1°±0.2.

6. The crystalline solid form of any one of claims 1-5, wherein the crystalline solid form is characterized by an XRPD pattern having characteristic peaks at the diffraction angles (2θ) at 5.9°±0.2, 17.6°±0.2, 21.4°±0.2, 23.1°±0.2, and 25.1°±0.2.

7. The crystalline solid form of any one of claims 1-6, wherein the crystalline solid form is characterized by an XRPD pattern substantially similar to FIG. 1.

8. The crystalline solid form of any one of claims 1-7, wherein the crystalline solid form is characterized by differential scanning calorimetry (DSC) having two endotherms with onset peaks at 186.2±2° C. and 197.5±2° C.

9. The crystalline solid form of any one of claims 1-8, obtained by a process comprising recrystallization of Compound (I) in a solvent mixture comprising acetone and n-heptane.

10. The crystalline solid form of claim 9, obtained by a process further comprising one or both of the following steps:
a. dissolving Compound (I) in acetone at an elevated temperature to form a solution; and
b. evaporative concentration and cooling crystallization in a solvent mixture comprising acetone and n-heptane.

11. A crystalline solid form of the free base of Compound (I), wherein Compound (I) is represented by the following structural formula: 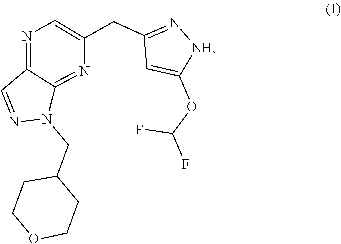 [SMILES: FC(F)Oc1cc(Cc2cnc3cnn(CC4CCOCC4)c3n2)n[nH]1]
wherein the crystalline solid form is referred to as crystalline solid Form B and is characterized by an XRPD pattern comprising three, four, or five peaks at the diffraction angles (2θ) selected from the group consisting of 5.8°±0.2, 14.9°±0.2, 17.3°±0.2, 23.9°±0.2, and 24.6°±0.2.

12. A crystalline solid form of the free base of Compound (I), wherein the crystalline solid form is referred to as crystalline solid Form B and is characterized by an XRPD pattern comprising three, four, or five peaks at the diffraction angles (2θ) selected from the group consisting of 5.8°±0.2, 17.3°±0.2, 21.5°±0.2, 25.3°±0.2, and 29.1°±0.2.

13. The crystalline solid form of claim 11 or 12, wherein the crystalline solid form is characterized by an XRPD pattern comprising three, four, five, six, seven, eight, nine, or ten peaks at the diffraction angles (2θ) selected from the group consisting of 5.8°±0.2, 14.9°±0.2, 17.3°±0.2, 19.5°±0.2, 21.0°±0.2, 21.5°±0.2, 23.9°±0.2, 24.6°±0.2, 25.3°±0.2, and 29.1°±0.2.

14. The crystalline solid form of any one of claims 11-13, wherein the crystalline solid form is characterized by an XRPD pattern comprising three, four, five, six, seven, eight, nine, ten, eleven, twelve, thirteen, fourteen, or fifteen peaks at the diffraction angles (2θ) selected from the group consisting of 5.8°±0.2, 12.6°±0.2, 14.9°±0.2, 17.3°±0.2, 17.8°±0.2, 18.6°±0.2, 19.5°±0.2, 21.0°±0.2, 21.5°±0.2, 23.3°±0.2, 23.9°±0.2, 24.6°±0.2, 25.3°±0.2, 28.9°±0.2, and 29.1°±0.2.

15. The crystalline solid form of any one of claims 11-14, wherein the crystalline solid form is characterized by an XRPD pattern having characteristic peaks at the diffraction angles (2θ) at 5.8°±0.2, 17.3°±0.2, and 25.3°±0.2.

16. The crystalline solid form of any one of claims 11-15, wherein the crystalline solid form is characterized by an XRPD pattern having characteristic peaks at the diffraction angles (2θ) at 5.8°±0.2, 17.3°±0.2, 21.5°±0.2, 25.3°±0.2, and 29.1°±0.2.

17. The crystalline solid form of any one of claims 11-16, wherein the crystalline solid form is characterized by an XRPD pattern substantially similar to FIG. 3.

18. The crystalline solid form of any one of claims 11-17, wherein the crystalline solid form is characterized by DSC having a broad endotherm with an onset peak at 198.5±2° C.

19. The crystalline solid form of any one of claims 11-18, obtained by a process comprising one or both of the following steps:
a. dissolving the Compound (I) in acetone at an elevated temperature to form a solution; and
b. evaporating the acetone.

20. A crystalline solid form of the free base of Compound (I) represented by the following structural formula: 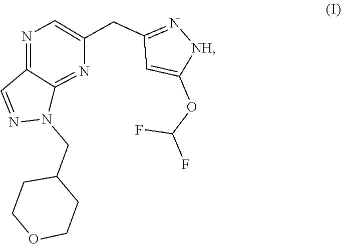 [SMILES: FC(F)Oc1cc(Cc2cnc3cnn(CC4CCOCC4)c3n2)n[nH]1]
wherein the crystalline solid form is referred to as crystalline solid Form C and is characterized by an XRPD pattern comprising three, four, five, or six peaks at the diffraction angles (2θ) selected from the group consisting of 8.7°±0.2, 10.6°±0.2, 13.4°±0.2, 15.3°±0.2, 19.0°±0.2, and 25.8°±0.2.

21. The crystalline solid form of claim 20, wherein the crystalline solid form is characterized by an XRPD pattern comprising three, four, five, six, seven, eight, nine, or ten peaks at the diffraction angles (2θ) selected from the group consisting of 8.7°±0.2, 10.6°±0.2, 13.4°±0.2, 15.3°±0.2, 16.2°±0.2, 19.0°±0.2, 19.8°±0.2, 21.4°±0.2, 22.6°±0.2, and 25.8°±0.2.

22. The crystalline solid form of claim 20 or 21, wherein the crystalline solid form is characterized by an XRPD pattern having characteristic peaks at the diffraction angles (2θ) at 8.7°±0.2, 15.3°±0.2, and 25.8°±0.2.

23. The crystalline solid form of any one of claims 20-22, wherein the crystalline solid form is characterized by an XRPD pattern having characteristic peaks at the diffraction angles (2θ) at 8.7°±0.2, 15.3°±0.2, 19.0°±0.2, 19.8°±0.2, and 25.8°±0.2.

24. The crystalline solid form of any one of claims 20-23, wherein the crystalline solid form is characterized by an XRPD pattern substantially similar to FIG. 5.

25. The free base of any one of claims 20-24, wherein the crystalline Form C is characterized by DSC having three broad endotherms with onset peaks at 30.6±2° C., 170.5±2° C., and 198.4±2° C. and two broad exotherms with onset peaks at 176.8±2° C. and 253.7±2° C.

26. The free base of any one of claims 20-25, obtained by a process comprising one or more steps selected from the group consisting of:
a. dissolution of Compound (I) in a solvent mixture comprising acetonitrile and water; and
b. lyophilization.

27. An amorphous form of the free base of Compound (I) represented by the following structural formula:

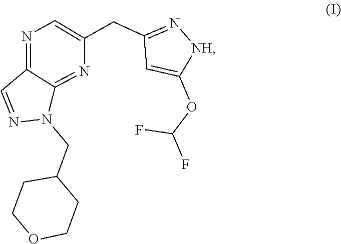 [SMILES: FC(F)Oc1cc(Cc2cnc3cnn(CC4CCOCC4)c3n2)n[nH]1]

28. A crystalline solid form of a pharmaceutically acceptable salt of Compound (I) represented by the following structural formula: 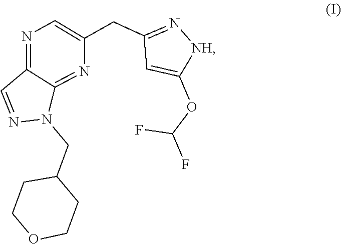 [SMILES: FC(F)Oc1cc(Cc2cnc3cnn(CC4CCOCC4)c3n2)n[nH]1]
wherein the salt is selected from the group consisting of a methanesulfonate salt, an ethanesulfonate salt, a benzenesulfonate salt, a tosylate salt, a naphthalene-2-sulfonate salt, a glycine salt, a succinate salt, a citrate salt, a tartrate salt, an adipate salt, an aspartate salt, a histidine salt, a vanillin salt, a phosphate salt, a hydrochloride salt, a hydrobromide salt, a nitrate salt, and a sulfate salt.

29. The salt form of claim 28, wherein the crystalline solid form is of a tosylate salt.

30. The salt of claim 29, wherein the crystalline solid form is referred to as crystalline tosylate salt Form 6-C and is characterized by one or both of the following:
a. an XRPD pattern substantially similar to FIG. 15; and
b. a DSC having an endotherm with an onset peak at 165.5±2° C.

31. The crystalline solid form of claim 28, wherein the crystalline solid form is of an ethanesulfonate salt.

32. The salt of claim 31, wherein the crystalline solid form is referred to as crystalline ethanesulfonate salt Form 7-A and is characterized by one or both of the following:
a. an XRPD pattern substantially similar to FIG. 19; and
b. a DSC having a broad endotherm with an onset peak at 183.8±2° C.

33. The crystalline solid form of claim 28, wherein the crystalline solid form is of a naphthalene-2-sulfonate salt.

34. The crystalline solid form of claim 33, wherein the crystalline solid form is referred to as crystalline naphthalene-2-sulfonate salt Form 8-A and is characterized by one or both of the following:
a. an XRPD pattern substantially similar to FIG. 21; and
b. a DSC having a broad endotherm with an onset peak at 199.9±2° C.

35. The crystalline solid form of claim 28, wherein the crystalline solid form is of a hydrochloride salt.

36. The crystalline solid form of claim 35, wherein the crystalline solid form is referred to as crystalline hydrochloride salt Form 1-B and is characterized by one or both of the following:
a. an XRPD pattern substantially similar to FIG. 23; and
b. a DSC having a broad endotherm with an onset peak at 199.7±2° C.

37. A pharmaceutical composition comprising the crystalline solid form of the free base of Compound (I) of any one of claims 1-26, the amorphous form of the free base of Compound (I) of claim 27, or the crystalline solid form of a pharmaceutically acceptable salt of Compound (I) of any one of claims 28-36, and a pharmaceutically acceptable carrier.

38. A pharmaceutical composition comprising Compound (I) or a pharmaceutically acceptable salt thereof and a pharmaceutically acceptable carrier, wherein at least 0.1%, 0.5%, 1%, 5%, or more of the Compound (I) or pharmaceutically acceptable salt thereof by weight is present in the crystalline solid form of the free base of Compound (I) of any one of claims 1-26, the amorphous form of the free base of Compound (I) of claim 27, or the crystalline solid form of a pharmaceutically acceptable salt of Compound (I) of any one of claims 28-36.

39. A method of treating a cancer, comprising administering a subject in need thereof an effective amount of the crystalline solid form of the free base of Compound (I) of any one of claims 1-26, the amorphous form of the free base of Compound (I) of claim 27, or the crystalline solid form of a pharmaceutically acceptable salt of Compound (I) of any one of claims 28-36, or a pharmaceutical composition of claim 37 or 38.

40. The method of claim 39, wherein the cancer is breast cancer.

41. The method of claim 39, wherein the cancer is selected from the group consisting of ovarian cancer, endometrial cancer, gastric cancer, esophageal cancer, triple negative breast cancer, and lung adenosarcoma.

42. A method of treating a patient having an amplified expression level of CCNE1 and suffering from, or at risk of developing, a solid tumor cancer, comprising administering to the patient a therapeutically effective amount of the crystalline solid form of the free base of Compound (I) of any one of claims 1-26, the amorphous form of the free base of Compound (I) of claim 27, or the crystalline solid form of a pharmaceutically acceptable salt of Compound (I) of any one of claims 28-36, or a pharmaceutical composition of claim 37 or 38.

43. The method of claim 42, wherein the solid tumor cancer is at least one of uterine cancer (including uterine carcinosarcoma, uterine corpus endometrial carcinoma), endometrial cancer, breast cancer (including breast invasive carcinoma, TNBC (triple negative breast cancer), ER (estrogen receptor)+HER2 (human epidermal growth factor 2)− breast cancer, and HER2+ breast cancer), ovarian cancer (e.g. ovarian serous cystadenocarcinoma), stomach cancer (including stomach adenocarcinoma), gastric cancer (including gastrointestinal stromal tumor), colorectal cancer, pancreatic cancer, kidney cancer, head and neck cancer, liver cancer, prostate cancer, skin cancer, lymphoma (including B-cell lymphoma), sarcoma, esophageal cancer (including esophageal carcinoma), bladder cancer (including bladder urothelial carcinoma), lung cancer (including lung squamous carcinoma and non-small cell lung cancer, e.g., EGFRm (epidermal growth factor receptor mutant)+non-small cell lung cancer), cholangiocarcinoma, adrenocortical carcinoma, or mesothelioma.

44. A method of preparing Compound (I): 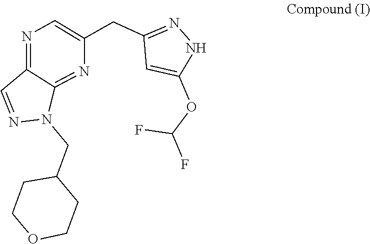 [SMILES: FC(F)Oc1cc(Cc2cnc3cnn(CC4CCOCC4)c3n2)n[nH]1]
comprising reacting a first compound represented by formula (1): 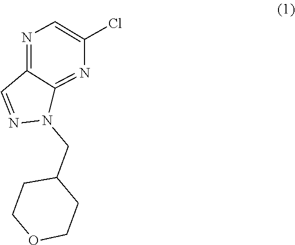 [SMILES: Clc1cnc2cnn(CC3CCOCC3)c2n1]
and a second compound represented by formula (2): 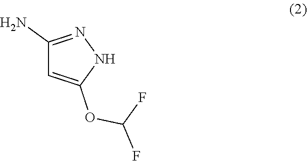
wherein reacting the first and second compound further includes a base that activates the diamine compound (2) that then reacts with the heteroaryl chloride compound (1).

45. The method of claim 44, wherein the base is a strong non-nucleophilic amide base.

46. The method of claim 45, wherein the strong non-nucleophilic amide base is LiHMDS (lithium bis(trimethylsilyl)amide), NaHMDS (sodium bis(trimethylsilyl)amide), KHMDS (potassium bis(trimethylsilyl)amide), or lithium diisopropylamide (LDA).
